(19) United States
(12) Patent Application Publication
(10) Pub. No.: US 2025/0281457 A1
(43) Pub. Date: Sep. 11, 2025

(54) TREATMENT OR PREVENTION OF DENGUE VIRAL INFECTION

(71) Applicant: Janssen Pharmaceuticals, Inc., Titusville, NJ (US)

(72) Inventors: Oliver Ackaert, Essen (BE); Tristan Baguet, Steenhuffel (BE); Claire Balmain, Antwerpen (BE); Nele Goeyvaerts, Kuringen (BE); Guillermo Herrera-Taracena, Malvern, PA (US); Dymphy Huntjens, Broekhem (NL); Freya Rasschaert, Vissenaken (BE); Bram Schroyen, Antwerp (BE); Ivan Somers, Herk-de-stad (BE)

(21) Appl. No.: 18/862,833

(22) PCT Filed: May 1, 2023
(86) PCT No.: PCT/IB2023/054527

§ 371 Date: Nov. 4, 2024

Related U.S. Application Data
(60) Provisional application No. 63/341,074, filed on May 12, 2022.

(51) Int. Cl. A61K 31/4045; A61K 9/20

(52) U.S. Cl. CPC A61K 31/4045; A61K 9/2027; A61K 9/2054

(57) ABSTRACT

The present invention relates to a compound of formula (I) for use in the prevention and/or treatment of dengue viral infections, wherein the compound of formula (I) is administered in an oral dosage form to a human in either a fed or fasted state and wherein said compound of formula (I) corresponds to

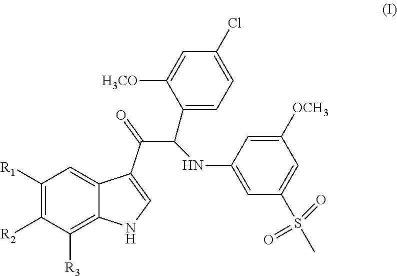

a stereo-isomeric form, a pharmaceutically acceptable salt, solvate or polymorph thereof; said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃.

### CROSS REFERENCE TO RELATED APPLICATIONS

This application claims priority to U.S. Provisional Patent Application No. 63/341,074 filed on May 12, 2022, the entire content of which is expressly incorporated herein by reference in its entirety.

### TECHNICAL FIELD

The present invention relates to the use of substituted indole derivatives in the manufacture of a pharmaceutical formulation for the treatment and/or the prevention of dengue viral infection and the administration of said pharmaceutical formulation to a subject in need thereof in either a fed or a fasted state. The invention further provides a method for the treatment and/or the prevention of dengue viral infection.

### BACKGROUND OF THE INVENTION

Dengue is caused by any of the 4 antigenically distinct DENV serotypes (DENV-1, -2, -3, and -4), which belong to the genus Flavivirus in the family of the Flaviviridae. The DENVs are human pathogens which are transmitted through the bite of an infected female mosquito of the genus Aedes. Dengue is endemic in more than 125 countries, and has also again become endemic in the United States (US) territories of Puerto Rico, American Samoa, and the Virgin Islands. About half of the global population is currently at risk of becoming infected with DENV. According to the World Health Organization (WHO), dengue is among the top 10 threats to global health in 2019.

Dengue is widespread throughout the tropics, with the local variations in risk influenced by rainfall, temperature, and rapid urbanization. The exponential growth of the number of dengue cases reflects the global spread of the mosquito vectors, which is driven by climate changes and, more importantly, human population growth, urbanization, and globalization.

The actual numbers of dengue cases are underreported, and many cases are misclassified as other febrile illnesses such as malaria. It is estimated that there are 390 million DENV infections globally per year of which 96 million infections manifest clinically (with any severity of the disease). On average, each year about 500,000 dengue cases require hospitalization due to severe and life-threatening disease and up to 25,000 patients die due to dengue. There is, therefore, a need to develop an anti-DENV molecule for the prevention and treatment of dengue.

Currently, there is no dengue-specific treatment available and thus, clinical treatment is principally supportive in nature.

During recent years, drugs developed for prophylactic use are slowly getting more attention as potential alternatives to prevent dengue. Prophylaxis could be beneficial for travelers to dengue-endemic regions (e.g., aid workers, tourists, business and military travelers, and expatriates), as well as for vulnerable populations living in endemic regions. By preventing viremia and/or by reducing viral load, dengue-associated morbidity and mortality could be reduced remarkably or even prevented. In addition, an efficacious and safe dengue antiviral compound could still have its use as a therapeutic agent as well.

WO 2016/180696 discloses compounds for the prevention and treatment of dengue viral infections. There is, however, a great unmet medical need for medicaments allowing the treatment and/or the prevention of dengue disease (also called dengue) in animals, more in particular in humans.

In prophylaxis, the dosing regimen should be as lean as possible and as safe as possible ensuring maximal therapy compliance. For instance, daily dosing (QD) has several advantages, such as a lower peak-to-trough ratio, the effect of the intake of food becomes less critical, and the pill burden to patients may be lower; however, QD may have limitations on the therapy compliance. Weekly dosing (Q7D) may be a viable option with potentially advantageous therapy compliance, although it may be associated with certain limitations, such as a higher peak-to-trough ratio, potentially a higher pill burden to patients, and potentially a greater role in the intake of food.

### SUMMARY OF THE INVENTION

The present invention is directed to a compound of formula (I) for use in the prevention and/or treatment of dengue viral infections, wherein the compound of formula (I) is administered in an oral dosage form to a human subject in either a fed or fasted state, wherein said compound of formula (I) corresponds to

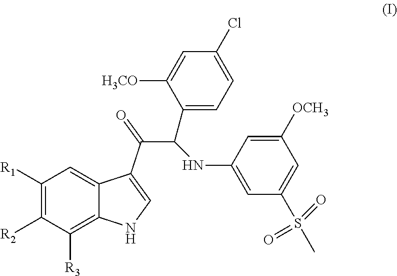

a stereo-isomeric form, a pharmaceutically acceptable salt, solvate or polymorph thereof;
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃.

Embodiments of the invention include the compound being administered as an oral dosage form to a human in a fed state; preferably wherein said human is in a fed state before the administration of said oral dosage form or simultaneously with the administration of said oral dosage form.

Further embodiments of the invention include the compound being administered as a solid dosage form comprising a pharmaceutical formulation, said formulation preferably comprising a cellulose derivative such as hydroxypropyl methylcellulose (HPMC), or methacrylic acid copolymer or a combination thereof.

The invention also provides a pharmaceutical formulation, for use in the prevention and/or treatment of dengue viral infection, said formulation comprising
a) a compound formula (I) as described hereinabove; and
b1) methacrylic acid copolymer, or
b2) a cellulose derivative such as hydroxypropyl methylcellulose (HPMC);
wherein the pharmaceutical formulation is administered in an oral dosage form to a human in either a fed or fasted state. Said human is at risk of being infected by Dengue virus or infected by Dengue virus.

The present invention is also directed to such pharmaceutical formulations and/or solid dosage form for use in the treatment and/or prevention of dengue viral infections.

### BRIEF DESCRIPTION OF DRAWINGS

The following detailed description, given by way of example, but not intended to limit the invention solely to the specific embodiments described, may best be understood in conjunction with the accompanying drawings.

FIG. 1 represents a boxplot of the AUCₗₐₛₜ (ng h/mL) of single 400 mg dose of compound (a) according to the invention, as a function of treatment under fasting conditions: oral solution (Treatment A) or SF1b (Treatment B).

FIG. 2 represents a boxplot of the AUCₗₐₛₜ (ng h/mL) of single 400 mg dose of compound (a) according to the invention, as a function of treatment under fasting conditions: oral solution (Treatment A) or SF2b (Treatment C).

FIG. 3 represents a boxplot of the AUCₗₐₛₜ (ng h/mL) of single 800 mg dose of compound (a) (SF2a/b) according to the invention, as a function of treatment under fasting or fed conditions: fasting conditions (Treatments D and J), standardized breakfast (Treatment E), high-fat, high-calorie breakfast (Treatment K), low-fat, low calorie breakfast (Treatment L), low-fat, high calorie breakfast (Treatment M).

FIG. 4 represents a boxplot of the AUCₗₐₛₜ (ng h/mL) of single 200 mg dose of compound (a) (SF2a/b) according to the invention, as a function of treatment under fasting or fed conditions: fasting conditions (Treatment F), standardized breakfast (Treatment G), low-fat, low calorie breakfast (Treatment N), low-fat, high calorie breakfast (Treatment O).

FIG. 5 represents a graph plotting the mean (SD) plasma concentrations of Compound (a) (ng/mL) up to 12 hours on Day 1 of treatment. Panel I corresponds to a loading dose (LD) of 450 mg Compound (a) administered twice daily (BID) on Days 1 and 2, followed by a maintenance dose (MD) of 900 mg once a week (Q7D) on Days 3, 10, 17 and 24. Panel III corresponds to a loading dose of 150 mg Compound (a) administered twice daily (BID) on Days 1 and 2, followed by a maintenance dose of 300 mg once a week (Q7D) on Days 3, 10, 17 and 24.

FIG. 6 represents a graph plotting the mean (SD) plasma concentrations of Compound (a) (ng/ml) up to 12 hours on Day 2 of treatment. Panel I corresponds to a LD of 450 mg Compound (a) administered twice daily (BID) on Days 1 and 2, followed by a MD of 900 mg once a week on Days 3, 10, 17 and 24. Panel III corresponds to a LD of 150 mg Compound (a) administered twice daily (BID) on Days 1 and 2, followed by a MD of 300 mg once a week (Q7D) on Days 3, 10, 17 and 24.

FIG. 7 represents a graph plotting the mean (SD) plasma concentrations of Compound (a) (ng/mL) up to 12 hours on Day 3 of treatment. Panel I corresponds to a LD of 450 mg Compound (a) administered twice daily (BID) on Days 1 and 2, followed by a MD of 900 mg once a week on Days 3, 10, 17 and 24. Panel III corresponds to a LD of 150 mg Compound (a) administered twice daily (BID) on Days 1 and 2, followed by a MD of 300 mg once a week (Q7D) on Days 3, 10, 17 and 24.

FIG. 8 represents a graph plotting the mean (SD) plasma concentrations of Compound (a) (ng/mL) up to 72 hours on Days 3 to 6 of treatment. Panel I corresponds to a LD of 450 mg Compound (a) administered twice daily (BID) on Days 1 and 2, followed by a MD of 900 mg once a week on Days 3, 10, 17 and 24. Panel III corresponds to a LD of 150 mg Compound (a) administered twice daily (BID) on Days 1 and 2, followed by a MD of 300 mg once a week (Q7D) on Days 3, 10, 17 and 24.

FIG. 9 represents a graph plotting the mean (SD) plasma concentrations of Compound (a) (ng/mL) up to 12 hours on Day 24 of treatment. Panel I corresponds to a LD of 450 mg Compound (a) administered twice daily (BID) on Days 1 and 2, followed by a MD of 900 mg once a week on Days 3, 10, 17 and 24. Panel III corresponds to a LD of 150 mg Compound (a) administered twice daily (BID) on Days 1 and 2, followed by a MD of 300 mg once a week (Q7D) on Days 3, 10, 17 and 24.

FIG. 10 represents a graph plotting the mean (SD) plasma concentrations of Compound (a) (ng/mL) up to 840 hours, Days 24 to 59 of treatment. Panel I corresponds to a LD of 450 mg Compound (a) administered twice daily (BID) on Days 1 and 2, followed by a MD of 900 mg once a week (Q7D) on Days 3, 10, 17 and 24. Panel III corresponds to a LD of 150 mg Compound (a) administered twice daily (BID) on Days 1 and 2, followed by a MD of 300 mg once a week (Q7D) on Days 3, 10, 17 and 24.

FIG. 11 represents a graph plotting the mean (SD) plasma concentrations of Compound (a) (ng/mL) up to 12 hours on Day 1 of treatment. Panel II corresponds to a LD of 450 mg Compound (a) administered twice daily (BID) on Days 1 and 2, followed by a MD of 450 mg once every 3 (Q3D) or 4 days (Q4D) on Days 3, 6, 10, 13, 17, 20, 24 and 27. Panel IV corresponds to a LD of 150 mg Compound (a) administered twice daily (BID) on Days 1 and 2, followed by a MD of 150 mg once every 3 (Q3D) or 4 days (Q4D) on Days 3, 6, 10, 13, 17, 20, 24 and 27.

FIG. 12 represents a graph plotting the mean (SD) plasma concentrations of Compound (a) (ng/ml) up to 12 hours on Day 2 of treatment. Panel II corresponds to a LD of 450 mg Compound (a) administered twice daily (BID) on Days 1 and 2, followed by a MD of 450 mg once every 3 (Q3D) or 4 days (Q4D) on Days 3, 6, 10, 13, 17, 20, 24 and 27. Panel IV corresponds to a LD of 150 mg Compound (a) administered twice daily (BID) on Days 1 and 2, followed by a MD of 150 mg once every 3 (Q3D) or 4 days (Q4D) on Days 3, 6, 10, 13, 17, 20, 24 and 27.

FIG. 13 represents a graph plotting the mean (SD) plasma concentrations of Compound (a) (ng/mL) up to 12 hours on Day 3 of treatment. Panel II corresponds to a LD of 450 mg Compound (a) administered twice daily (BID) on Days 1 and 2, followed by a MD of 450 mg once every 3 (Q3D) or 4 days (Q4D) on Days 3, 6, 10, 13, 17, 20, 24 and 27. Panel IV corresponds to a LD of 150 mg Compound (a) administered twice daily (BID) on Days 1 and 2, followed by a MD of 150 mg once every 3 (Q3D) or 4 days (Q4D) on Days 3, 6, 10, 13, 17, 20, 24 and 27.

FIG. 14 represents a graph plotting the mean (SD) plasma concentrations of Compound (a) (ng/mL) up to 72 hours on Days 3 to 6 of treatment. Panel II corresponds to a LD of 450 mg Compound (a) administered twice daily (BID) on Days 1 and 2, followed by a MD of 450 mg once every 3 (Q3D) or 4 days (Q4D) on Days 3, 6, 10, 13, 17, 20, 24 and 27. Panel IV corresponds to a LD of 150 mg Compound (a) administered twice daily (BID) on Days 1 and 2, followed by a MD of 150 mg once every 3 (Q3D) or 4 days (Q4D) on Days 3, 6, 10, 13, 17, 20, 24 and 27.

FIG. 15 represents a graph plotting the mean (SD) plasma concentrations of Compound (a) (ng/mL) up to 12 hours on Day 27 of treatment. Panel II corresponds to a LD of 450 mg Compound (a) administered twice daily (BID) on Days 1 and 2, followed by a MD of 450 mg once every 3 (Q3D) or 4 days (Q4D) on Days 3, 6, 10, 13, 17, 20, 24 and 27. Panel IV corresponds to a LD of 150 mg Compound (a) administered twice daily (BID) on Days 1 and 2, followed by a MD of 150 mg once every 3 (Q3D) or 4 days (Q4D) on Days 3, 6, 10, 13, 17, 20, 24 and 27.

FIG. 16 represents a graph plotting the mean (SD) plasma concentrations of Compound (a) (ng/mL) up to 840 hours on Days 27 to 62 of treatment. Panel II corresponds to a LD of 450 mg Compound (a) administered twice daily (BID) on Days 1 and 2, followed by a MD of 450 mg once every 3 (Q3D) or 4 days (Q4D) on Days 3, 6, 10, 13, 17, 20, 24 and 27. Panel IV corresponds to a LD of 150 mg Compound (a) administered twice daily (BID) on Days 1 and 2, followed by a MD of 150 mg once every 3 (Q3D) or 4 days (Q4D) on Days 3, 6, 10, 13, 17, 20, 24 and 27.

FIG. 17 represents graphs plotting the mean plasma concentrations of Compound (a) (ng/ml) against time (days).

First graph (entitled “450/900 BID2d/Q7D”) corresponds to Panel I: a LD of 450 mg Compound (a) administered twice daily (BID) on Days 1 and 2, followed by a MD of 900 mg once a week (Q7D) on Days 3, 10, 17 and 24.

Second graph (entitled “450/450 BID2d/Q3D-Q4D”) corresponds to Panel II: a LD of 450 mg Compound (a) administered twice daily (BID) on Days 1 and 2, followed by a MD of 450 mg once every 3 (Q3D) or 4 days (Q4D) on Days 3, 6, 10, 13, 17, 20, 24 and 27.

Third graph (entitled “150/300 BID2d/Q7D”) corresponds to Panel III: a LD of 150 mg Compound (a) administered twice daily (BID) on Days 1 and 2, followed by a MD of 300 mg once a week (Q7D) on Days 3, 10, 17 and 24.

Fourth graph (entitled “150/150 BID2d/Q3D-Q4D”) corresponds to Panel IV: a LD of 150 mg Compound (a) administered twice daily (BID) on Days 1 and 2, followed by a MD of 150 mg once every 3 (Q3D) or 4 days (Q4D) on Days 3, 6, 10, 13, 17, 20, 24 and 27.

### DETAILED DESCRIPTION OF THE INVENTION

The disclosure may be more fully appreciated by reference to the following description, including the following glossary of terms and the concluding examples. It is to be appreciated that certain features of the disclosed pharmaceutical formulations and methods which are, for clarity, described herein in the context of separate aspects, may also be provided in combination in a single aspect. Conversely, various features of the disclosed pharmaceutical formulations and methods that are, for brevity, described in the context of a single aspect, may also be provided separately or in any sub-combination.

Some of the quantitative expressions given herein are not qualified with the term “about” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The terms “about” or “approximately” as used herein, when referring to a numerical value or range, allow for a degree of variability in the value or range, for example, within 10% (i.e., ±10%), within 5% (i.e., ±5%), or within 2.5% (i.e., ±2.5%) of a stated value or of a stated limit of a range.

Throughout the description and claims of this specification, the words “comprise” and “contain” and variations of the words, for example “comprising” and “comprises”, mean “including but not limited to”, and are not intended to (and do not) exclude other components.

The recitation of numerical ranges by endpoints includes all integer numbers and, where appropriate, fractions subsumed within that range (e.g., 1 to 5 can include 1, 2, 3, 4 when referring to, for example, a number of elements, and can also include 1.5, 2, 2.75 and 3.80, when referring to, for example, measurements). The recitation of end points also includes the end point values themselves (e.g., from 1.0 to 5.0 includes both 1.0 and 5.0). Any numerical range recited herein is intended to include all sub-ranges subsumed therein.

All references cited in the present specification are hereby incorporated by reference in their entirety. In particular, the teachings of all references herein specifically referred to are incorporated by reference.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art.

As used herein, the term “treat”, “treating”, or “treatment” of any disease, condition, syndrome or disorder refers, in one embodiment, to ameliorating the disease, condition, syndrome or disorder (i.e. slowing or arresting or reducing the development of the disease or at least one of the clinical symptoms thereof). In another embodiment, “treat”, “treating”, or “treatment” refers to alleviating or ameliorating at least one physical parameter including those which may not be discernible by the patient. In a further embodiment, “treat”, “treating”, or “treatment” refers to modulating the disease, condition, syndrome or disorder either physically (e.g., stabilization of a discernible symptom), physiologically, (e.g., stabilization of a physical parameter), or both. In yet another embodiment, “treat”, “treating”, or “treatment” refers to preventing or delaying the onset or development or progression of the disease, condition, syndrome or disorder.

As used herein, the terms “prevent”, “prevention” and “preventing” refer to the reduction in the risk of acquiring or developing a given disease, condition, syndrome, or disorder or the reduction or inhibition of the recurrence or said disease, condition, syndrome, or disorder in a subject who is not ill, but who has been or may be near a person with the disease, condition, syndrome, or disorder.

As used herein, the term “Dengue virus” refers to the single positive-stranded RNA virus of the family Flaviviridae; four distinct, but closely related serotypes of the flavivirus dengue are known, so-called DENV-1, -2, -3, and -4. Flaviviruses, which are transmitted by mosquitoes or ticks, cause life-threatening infections in man, such as encephalitis and hemorrhagic fever.

As used herein, the term “dengue viral infection” refers to at least one condition, syndrome, symptom, disorder, and/or disease caused by a Dengue virus.

The term “fasted state” or “fasted condition” is used herein to refer to a subject, preferably a human subject, which has not eaten for at least 4 hours before a time point of interest, such as the time of administering a compound of formula (I). In an embodiment, a subject, preferably a human subject, in the fasted state has not eaten for at least 0.25 hour (h), preferably for at least 0.5 h, preferably for at least 1 h, preferably for at least 2 h, preferably for at least 3 h, preferably for at least 5 h, preferably for at least 6 h, preferably for at least 7 h, preferably for at least 8 h, preferably for at least 9 h, preferably for at least 10 h, preferably for at least 11 h, preferably for at least 12 h, preferably at least 8 h to at most 12 h, more preferably at least 10 h, or any sub-range therein, prior to the administration of a compound of formula (I). In an embodiment, a subject, preferably a human subject, in the fasted state has not eaten for at least 0.25 hour (h), 0.5 h, 1 h, 2 h, 3 h, 5 h, 6 h, 7 h, 8 h, 9 h, 10 h, 11 h or 12 h, preferably at least 8 h to 12 h, more preferably at least 10 h, or any sub-range therein, prior to the administration of a compound of formula (I). In some embodiments, “fasted state” means that a subject, preferably a human subject, has not ingested more than 10 kilocalories, preferably not more than 20 kilocalories, preferably not more than 30 kilocalories, preferably not more than 40 kilocalories, preferably not more than 50 kilocalories (kcal, Calories, Cal), or any sub-range therein, or more than 10 kcal, preferably not more than 20 kcal, preferably not more than 30 kcal, preferably not more than 40 kcal, preferably not more than 50 kcal for at least 0.25 hour (h), preferably for at least 0.5 h, preferably for at least 1 h, preferably for at least 2 h, preferably for at least 3 h, preferably for at least 5 h, preferably for at least 6 h, preferably for at least 7 h, preferably for at least 8 h, preferably for at least 9 h, preferably for at least 10 h, preferably for at least 11 h, preferably for at least 12 h, preferably at least 8 h to at most 12 h, more preferably at least 10 h, prior to the administration of a compound of formula (I). In some embodiments, “fasted state” means that a subject, preferably a human subject, has not ingested 10, 20, 30, 40 or 50 kilocalories (kcal, Calories, Cal), or any sub-range therein, or more than 10, 20, 30, 40 or 50 kcal for at least 0.25 hour (h), 0.5 h, 1 h, 2 h, 3 h, 5 h, 6 h, 7 h, 8 h, 9 h, 10 h, 11 h or 12 h, preferably at least 8 h to 12 h, more preferably at least 10 h, prior to the administration of a compound of formula (I). In some embodiments, “fasted state” means that a subject, preferably a human subject, has ingested at most 10 kcal, preferably at most 20 kcal, preferably at most 30 kcal, preferably at most 40 kcal, preferably at most 50 kcal, preferably at most 100 kcal, preferably at most 150 kcal, preferably at most 200 kcal, preferably at most 250 kcal, preferably at most 300 kcal or any-sub range therein, at least 0.25 hour (h), preferably at least 0.5 h, preferably at least 1 h, preferably at least 2 h, preferably at least 3 h, preferably at least 5 h, preferably at least 6 h, preferably at least 7 h, preferably at least 8 h, preferably at least 9 h, preferably at least 10 h, preferably at least 11 h, preferably at least 12 h, preferably at least 8 h to at most 12 h, more preferably at least 10 h, or any sub-range therein, prior to the administration of a compound of formula (I). In some embodiments, “fasted state” means that a subject, preferably a human subject, has ingested at most 10, 20, 30, 40, 50, 100, 150, 200, 250, 300 kcal or any-sub range therein, at least 0.25 hour (h), 0.5 h, 1 h, 2 h, 3 h, 5 h, 6 h, 7 h, 8 h, 9 h, 10 h, 11 h or 12 h, preferably at least 8 h to 12 h, more preferably at least 10 h, or any sub-range therein, prior to the administration of a compound of formula (I). In some embodiments, “fasted state” means that a subject, preferably a human subject, has ingested at most 10 kcal, preferably at most 20 kcal, preferably at most 30 kcal, preferably at most 40 kcal, preferably at most 50 kcal, preferably at most 100 kcal, preferably at most 150 kcal, preferably at most 200 kcal, preferably at most 250 kcal, preferably at most 300 kcal or any-sub range therein, and at most 0.1 g of fat, preferably at most 0.2 g, preferably at most 0.3 g, preferably at most 0.4 g, preferably at most 0.5 g, preferably at most 0.6 g, preferably at most 0.7 g, preferably at most 0.8 g, preferably at most 0.9 g of fat; or at most 1 g of fat, preferably at most 2 g, preferably at most 3 g, preferably at most 4 g, preferably at most 5 g of fat; or at most 6 g of fat, preferably at most 7 g, preferably at most 8 g, preferably at most 9 g, preferably at most 10 g of fat; or at most 11 g of fat, for example at most 12 g, for example at most 13 g, for example at most 14 g, for example at most 15 g of fat; or at most 16 g of fat, for example at most 17 g, for example at most 18 g, for example at most 19 g, for example at most 20 g of fat; or at most 21 g fat, for example at most 22 g, for example at most 23 g, for example at most 24 g, for example at most 25 g of fat; or at most 26 g of fat, for example at most 27 g, for example at most 28 g, for example at most 29 g, for example at most 30 g of fat; or at most 31 g of fat, for example at most 32 g, for example at most 33 g, for example at most 34 g, for example at most 35 g of fat; or at most 36 g of fat, for example at most 37 g, for example at most 38 g, for example at most 39 g, for example at most 40 g of fat; or at most 41 g of fat, for example at most 42 g, for example at most 43 g, for example at most 44 g, for example at most 45 g of fat; or any particular amount or range comprised therein, at least 0.25 hour (h), preferably at least 0.5 h, preferably at least 1 h, preferably at least 2 h, preferably at least 3 h, preferably at least 5 h, preferably at least 6 h, preferably at least 7 h, preferably at least 8 h, preferably at least 9 h, preferably at least 10 h, preferably at least 11 h preferably at least 12 h, preferably at least 8 h to at most 12 h, more preferably at least 10 h, or any sub-range therein, prior to the administration of a compound of formula (I). In some embodiments, “fasted state” means that a subject, preferably a human subject, has ingested at most 10, 20, 30, 40, 50, 100, 150, 200, 250, 300 kcal or any-sub range therein, and at most 0.1 g, 0.2 g, 0.3 g, 0.4 g, 0.5 g, 0.6 g, 0.7 g, 0.8 g or 0.9 g of fat; or at most 1 g, 2 g, 3 g, 4 g or 5 g of fat; or at most 6 g, 7 g, 8 g, 9 g or 10 g of fat; or at most 11 g, 12 g, 13 g, 14 g or 15 g of fat; or at most 16 g, 17 g, 18 g, 18 g, 19 g or 20 g of fat; or at most 21 g, 22 g, 23 g, 24 g or 25 g of fat; or at most 26 g, 27 g, 28 g, 29 g or 30 g of fat; or at most 31 g, 32 g, 33 g, 34 g, or 35 g of fat; or at most 36 g, 37 g, 38 g, 39 g, or 40 g of fat; or at most 41 g, 42 g, 43 g, 44 g, or 45 g of fat; or any particular amount or range comprised therein, at least 0.25 hour (h), 0.5 h, 1 h, 2 h, 3 h, 5 h, 6 h, 7 h, 8 h, 9 h, 10 h, 11 h or 12 h, preferably at least 8 h to 12 h, more preferably at least 10 h, or any sub-range therein, prior to the administration of a compound of formula (I). In some embodiments, “fasted state” means that a subject, preferably a human subject, has ingested at most 10 kcal, preferably at most 20 kcal, preferably at most 30 kcal, preferably at most 40 kcal, preferably at most 50 kcal, preferably at most 100 kcal, preferably at most 150 kcal, preferably at most 200 kcal, preferably at most 250 kcal, preferably at most 300 kcal or any-sub range therein, and at most 0.1 g of fat, preferably at most 0.2 g, preferably at most 0.3 g, preferably at most 0.4 g, preferably at most 0.5 g, preferably at most 0.6 g, preferably at most 0.7 g, preferably at most 0.8 g preferably at most 0.9 g of fat; or at most 1 g of fat, preferably at most 2 g, preferably at most 3 g, preferably at most 4 g preferably at most 5 g of fat; or at most 6 g of fat, preferably at most 7 g, preferably at most 8 g, preferably at most 9 g preferably at most 10 g of fat; or at most 11 g of fat, preferably at most 12 g, preferably at most 13 g, preferably at most 14 g preferably at most 15 g of fat; or at most 16 g of fat, preferably at most 17 g, preferably at most 18 g, preferably at most 19 g, preferably at most 20 g of fat; or at most 21 g of fat, preferably at most 22 g, preferably at most 23 g, preferably at most 24 g, preferably at most 25 g of fat; or any particular amount or range comprised therein, at least 0.25 hour (h), preferably at least 0.5 h, preferably at least 1 h, preferably at least 2 h, preferably at least 3 h, preferably at least 5 h, preferably at least 6 h, preferably at least 7 h, preferably at least 8 h, preferably at least 9 h, preferably at least 10 h, preferably at least 11 h, preferably at least 12 h, preferably at least 8 h to at most 12 h, more preferably at least 10 h, or any sub-range therein, prior to the administration of a compound of formula (I). In some embodiments, “fasted state” means that a subject, preferably a human subject, has ingested at most 10, 20, 30, 40, 50, 100, 150, 200, 250, 300 kcal or any-sub range therein, and at most 0.1 g, 0.2 g, 0.3 g, 0.4 g, 0.5 g, 0.6 g, 0.7 g, 0.8 g or 0.9 g of fat; or at most 1 g, 2 g, 3 g, 4 g or 5 g of fat; or at most 6 g, 7 g, 8 g, 9 g or 10 g of fat; or at most 11 g, 12 g, 13 g, 14 g or 15 g of fat; or at most 16 g, 17 g, 18 g, 18 g, 19 g or 20 g of fat; or at most 21 g, 22 g, 23 g, 24 g or 25 g of fat; or any particular amount or range comprised therein, at least 0.25 hour (h), 0.5 h, 1 h, 2 h, 3 h, 5 h, 6 h, 7 h, 8 h, 9 h, 10 h, 11 h or 12 h, preferably at least 8 h to 12 h, more preferably at least 10 h, or any sub-range therein, prior to the administration of a compound of formula (I). In some embodiments, “fasted state” means that a subject, preferably a human subject, has ingested at most 0.1 g of fat, preferably at most 0.2 g, preferably at most 0.3 g, preferably at most 0.4 g, preferably at most 0.5 g, preferably at most 0.6 g, preferably at most 0.7 g, preferably at most 0.8 g, preferably at most 0.9 g of fat; or at most 1 g of fat, preferably at most 2 g, preferably at most 3 g, preferably at most 4 g, preferably at most 5 g of fat; or at most 6 g of fat, preferably at most 7 g, preferably at most 8 g, preferably at most 9 g, preferably at most 10 g of fat; or at most 11 g of fat, preferably at most 12 g, preferably at most 13 g, preferably at most 14 g, preferably at most 15 g of fat; or at most 16 g of fat, preferably at most 17 g, preferably at most 18 g, preferably at most 19 g, preferably at most 20 g of fat; or at most 21 g of fat, preferably at most 22 g, preferably at most 23 g, preferably at most 24 g, preferably at most 25 g of fat; or any particular amount or range comprised therein, at least 0.25 hour (h), preferably at least 0.5 h, preferably at least 1 h, preferably at least 2 h, preferably at least 3 h, preferably at least 5 h, preferably at least 6 h, preferably at least 7 h, preferably at least 8 h, preferably at least 9 h, preferably at least 10 h, preferably at least 11 h, preferably at least 12 h, preferably at least 8 h to at most 12 h, more preferably at least 10 h, or any sub-range therein, prior to the administration of a compound of formula (I). In some embodiments, “fasted state” means that a subject, preferably a human subject, has ingested at most 0.1 g, 0.2 g, 0.3 g, 0.4 g, 0.5 g, 0.6 g, 0.7 g, 0.8 g or 0.9 g of fat; or at most 1 g, 2 g, 3 g, 4 g or 5 g of fat; or at most 6 g, 7 g, 8 g, 9 g or 10 g of fat; or at most 11 g, 12 g, 13 g, 14 g or 15 g of fat; or at most 16 g, 17 g, 18 g, 18 g, 19 g or 20 g of fat; or at most 21 g, 22 g, 23 g, 24 g or 25 g of fat; or any particular amount or range comprised therein, at least 0.25 hour (h), 0.5 h, 1 h, 2 h, 3 h, 5 h, 6 h, 7 h, 8 h, 9 h, 10 h, 11 h or 12 h, preferably at least 8 h to 12 h, more preferably at least 10 h, or any sub-range therein, prior to the administration of a compound of formula (I). In some embodiments, “fasted state” means that a subject, preferably a human subject, has ingested at most 0.1 g of fat, preferably at most 0.2 g, preferably at most 0.3 g, preferably at most 0.4 g, preferably at most 0.5 g, preferably at most 0.6 g, preferably at most 0.7 g, preferably at most 0.8 g, preferably at most 0.9 g of fat; or at most 1 g of fat, preferably at most 2 g, preferably at most 3 g, preferably at most 4 g, preferably at most 5 g of fat; or at most 6 g of fat, preferably at most 7 g, preferably at most 8 g, preferably at most 9 g, preferably at most 10 g of fat; or at most 11 g of fat, preferably at most 12 g, preferably at most 13 g, preferably at most 14 g, preferably at most 15 g of fat; or at most 16 g of fat, preferably at most 17 g, preferably at most 18 g, preferably at most 19 g, preferably at most 20 g of fat; or at most 21 g of fat, preferably at most 22 g, preferably at most 23 g, preferably at most 24 g, preferably at most 25 g of fat; or at most 26 g of fat, preferably at most 27 g, preferably at most 28 g, preferably at most 29 g, preferably at most 30 g of fat; or at most 31 g of fat, preferably at most 32 g, preferably at most 33 g, preferably at most 34 g, preferably at most 35 g of fat; or at most 36 g of fat, preferably at most 37 g, preferably at most 38 g, preferably at most 39 g, preferably at most 40 g of fat; or at most 41 g of fat, preferably at most 42 g, preferably at most 43 g, preferably at most 44 g, preferably at most 45 g of fat; or any particular amount or range comprised therein, at least 0.25 hour (h), preferably at least 0.5 h, preferably at least 1 h, preferably at least 2 h, preferably at least 3 h, preferably at least 5 h, preferably at least 6 h, preferably at least 7 h, preferably at least 8 h, preferably at least 9 h, preferably at least 10 h, preferably at least 11 h, preferably at least 12 h, preferably at least 8 h to at most 12 h, more preferably at least 10 h, or any sub-range therein, prior to the administration of a compound of formula (I). In some embodiments, “fasted state” means that a subject, preferably a human subject, has ingested at most 0.1 g, 0.2 g, 0.3 g, 0.4 g, 0.5 g, 0.6 g, 0.7 g, 0.8 g or 0.9 g of fat; or at most 1 g, 2 g, 3 g, 4 g or 5 g of fat; or at most 6 g, 7 g, 8 g, 9 g or 10 g of fat; or at most 11 g, 12 g, 13 g, 14 g or 15 g of fat; or at most 16 g, 17 g, 18 g, 18 g, 19 g or 20 g of fat; or at most 21 g, 22 g, 23 g, 24 g or 25 g of fat; or at most 26 g, 27 g, 28 g, 29 g or 30 g of fat; or at most 31 g, 32 g, 33 g, 34 g, or 35 g of fat; or at most 36 g, 37 g, 38 g, 39 g, or 40 g of fat; or at most 41 g, 42 g, 43 g, 44 g, or 45 g of fat; or any particular amount or range comprised therein, at least 0.25 hour (h), 0.5 h, 1 h, 2 h, 3 h, 5 h, 6 h, 7 h, 8 h, 9 h, 10 h, 11 h or 12 h, preferably at least 8 h to 12 h, more preferably at least 10 h, or any sub-range therein, prior to the administration of a compound of formula (I).

As used herein, the term “fed state” or “fed condition” is used herein to refer to a subject, preferably a human subject, which has eaten less than or at most 4 hours before a time point of interest, such as the time of administering a compound of formula (I). In an embodiment, a subject in the fed state has eaten for at most 5 minutes (min), preferably at most 10 min, preferably at most 0.25 hour (h), preferably at most 0.5 h, preferably at most 1 h, preferably at most 2 h, preferably at most 3 h, preferably at most 4 h, preferably at most 5 h, preferably at most 6 h, preferably at most 7 h, preferably at most 8 h, preferably at most 9 h, preferably at most 10 h, preferably at most 11 h, preferably at most 12 h, preferably from at least 0 min to at most 2 h, preferably from at least 5 min to at most 2 h, more preferably from at least 0.25 h to at most 1 h, prior to the administration of a compound of formula (I). In an embodiment, a subject in the fed state has eaten for at most 5 minutes (min), 10 min, 0.25 hour (h), 0.5 h, 1 h, 2 h, 3 h, 4 h, 5 h, 6 h, 7 h, 8 h, 9 h, 10 h, 11 h or 12 h, preferably 0 min to 2 h or 5 min to 2 h, more preferably 0.25 h to 1 h, prior to the administration of a compound of formula (I). In some embodiments, the term “fed state” means that a human subject who has ingested at least 10 kcal, preferably at least 20 kcal, preferably at least 30 kcal, preferably at least 40 kcal, preferably at least 50 kcal, preferably at least 100 kcal, preferably at least 150 kcal, preferably at least 200 kcal, preferably at least 250 kcal, preferably at least 300 kcal, preferably at least 350 kcal, preferably at least 400 kcal, preferably at least 450 kcal, preferably at least 500 kcal or more, or any-sub range therein, for example at most 5 minutes (min), preferably at most 10 min, preferably at most 0.25 hour (h), preferably at most 0.5 h, preferably at most 1 h, preferably at most 2 h, preferably at most 3 h, preferably at most 4 h, preferably at most 5 h, preferably at most 6 h, preferably at most 7 h, preferably at most 8 h, preferably at most 9 h, preferably at most 10 h, preferably at most 11 h, preferably at most 12 h, preferably from at least 0 min to at most 2 h, preferably from at least 5 min to at most 2 h, more preferably from at least 0.25 h to at most 1 h, prior to the administration of a compound of formula (I). In some embodiments, the term “fed state” means that a human subject who has ingested at least 10, 20, 30, 40, 50, 100, 150, 200, 250, 300, 350, 400, 450, or 500 kcal or more, or any-sub range therein, for example at most 5 minutes (min), 10 min, 0.25 hour (h), 0.5 h, 1 h, 2 h, 3 h, 4 h, 5 h, 6 h, 7 h, 8 h, 9 h, 10 h, 11 h or 12 h, preferably 0 min to 2 h or 5 min to 2 h, more preferably 0.25 h to 1 h, prior to the administration of a compound of formula (I). In some embodiments, the term “fed state” means a subject, preferably a human subject, who has ingested at least 0.1 g of fat, preferably at least 0.2 g, preferably at least 0.3 g, preferably at least 0.4 g, preferably at least 0.5 g, preferably at least 0.6 g, preferably at least 0.7 g, preferably at least 0.8 g, preferably at least 0.9 g of fat, preferably at least 1 g of fat, preferably at least 2 g, preferably at least 3 g, preferably at least 4 g, preferably at least 5 g of fat, preferably at least 6 g of fat, preferably at least 7 g, preferably at least 8 g, preferably at least 9 g, preferably at least 10 g of fat, preferably at least 11 g of fat, preferably at least 12 g, preferably at least 13 g, preferably at least 14 g, preferably at least 15 g of fat; preferably at least 16 g of fat, preferably at least 17 g, preferably at least 18 g, preferably at least 19 g, preferably at least 20 g of fat; preferably at least 21 g of fat, preferably at least 22 g, preferably at least 23 g, preferably at least 24 g, preferably at least 25 g of fat; preferably at least 26 g of fat, preferably at least 27 g, preferably at least 28 g, preferably at least 29 g, preferably at least 30 g of fat; preferably at least 31 g of fat, preferably at least 32 g, preferably at least 33 g, preferably at least 34 g, preferably at least 35 g of fat; preferably at least 36 g of fat, preferably at least 37 g, preferably at least 38 g, preferably at least 39 g, preferably at least 40 g of fat; preferably at least 41 g of fat, preferably at least 42 g, preferably at least 43 g, preferably at least 44 g, preferably at least 45 g of fat; preferably at least 46 g of fat, preferably at least 47 g, preferably at least 48 g, preferably at least 49 g, preferably at least 50 g of fat; preferably at least 51 g of fat, 52 g, preferably at least 53 g, preferably at least 54 g, preferably at least 55 g of fat; preferably at least 56 g of fat, preferably at least 57 g, preferably at least 58 g, preferably at least 59 g, preferably at least 60 g of fat, or any particular amount or range comprised therein, for example, at most 5 minutes (min), preferably at most 10 min, preferably at most 0.25 hour (h), preferably at most 0.5 h, preferably at most 1 h, preferably at most 2 h, preferably at most 3 h, preferably at most 4 h, preferably at most 5 h, preferably at most 6 h, preferably at most 7 h, preferably at most 8 h, preferably at most 9 h, preferably at most 10 h, preferably at most 11 h, preferably at most 12 h, preferably from at least 0 min to at most 2 h, preferably from at least 5 min to at most 2 h, more preferably from at least 0.25 h to at most 1 h, prior to the administration of a compound of formula (I). In some embodiments, the term “fed state” means a subject, preferably a human subject, who has ingested at least 0.1 g, 0.2 g, 0.3 g, 0.4 g, 0.5 g, 0.6 g, 0.7 g, 0.8 g or 0.9 g of fat, preferably at least 1 g, 2 g, 3 g, 4 g or 5 g of fat, preferably at least 6 g, 7 g, 8 g, 9 g or 10 g of fat, preferably at least 11 g, 12 g, 13 g, 14 g or 15 g of fat; preferably at least 16 g, 17 g, 18 g, 18 g, 19 g or 20 g of fat; preferably at least 21 g, 22 g, 23 g, 24 g or 25 g of fat; preferably at least 26 g, 27 g, 28 g, 29 g or 30 g of fat; preferably at least 31 g, 32 g, 33 g, 34 g, or 35 g of fat; preferably at least 36 g, 37 g, 38 g, 39 g, or 40 g of fat; preferably at least 41 g, 42 g, 43 g, 44 g, or 45 g of fat; preferably at least 46 g, 47 g, 48 g, 49 g, or 50 g of fat; preferably at least 51 g, 52 g, 53 g, 54 g, or 55 g of fat; preferably at least 56 g, 57 g, 58 g, 59 g, or 60 g of fat, or any particular amount or range comprised therein, for example, at most 5 minutes (min), 10 min, 0.25 hour (h), 0.5 h, 1 h, 2 h, 3 h, 4 h, 5 h, 6 h, 7 h, 8 h, 9 h, 10 h, 11 h or 12 h, preferably 0 min to 2 h or 5 min to 2 h, more preferably 0.25 h to 1 h, prior to the administration of a compound of formula (I). In some embodiments, the term “fed state” means a subject, preferably a human subject, who has ingested at least 21 g of fat, preferably at least 22 g, preferably at least 23 g, preferably at least 24 g, preferably at least 25 g of fat; preferably at least 26 g of fat, preferably at least 27 g, preferably at least 28 g, preferably at least 29 g, preferably at least 30 g of fat; preferably at least 31 g of fat, preferably at least 32 g, preferably at least 33 g, preferably at least 34 g, preferably at least 35 g of fat; preferably at least 36 g of fat, preferably at least 37 g, preferably at least 38 g, preferably at least 39 g, preferably at least 40 g of fat; preferably at least 41 g of fat, preferably at least 42 g, preferably at least 43 g, preferably at least 44 g, preferably at least 45 g of fat; preferably at least 46 g of fat, preferably at least 47 g, preferably at least 48 g, preferably at least 49 g, preferably at least 50 g of fat; preferably at least 51 g of fat, preferably at least 52 g, preferably at least 53 g, preferably at least 54 g, preferably at least 55 g of fat; preferably at least 56 g of fat, preferably at least 57 g, preferably at least 58 g, preferably at least 59 g, preferably at least 60 g of fat, or any particular amount or range comprised therein, for example, at most 5 minutes (min), preferably at most 10 min, preferably at most 0.25 hour (h), preferably at most 0.5 h, preferably at most 1 h, preferably at most 2 h, preferably at most 3 h, preferably at most 4 h, preferably at most 5 h, preferably at most 6 h, preferably at most 7 h, preferably at most 8 h, preferably at most 9 h, preferably at most 10 h, preferably at most 11 h, preferably at most 12 h, preferably from at least 0 min to at most 2 h, preferably from at least 5 min to at most 2 h, more preferably from at least 0.25 h to at most 1 h, prior to the administration of a compound of formula (I). In some embodiments, the term “fed state” means a subject, preferably a human subject, who has ingested at least 21 g, 22 g, 23 g, 24 g or 25 g of fat; preferably at least 26 g, 27 g, 28 g, 29 g or 30 g of fat; preferably at least 31 g, 32 g, 33 g, 34 g, or 35 g of fat; preferably at least 36 g, 37 g, 38 g, 39 g, or 40 g of fat; preferably at least 41 g, 42 g, 43 g, 44 g, or 45 g of fat; preferably at least 46 g, 47 g, 48 g, 49 g, or 50 g of fat; preferably at least 51 g, 52 g, 53 g, 54 g, or 55 g of fat; preferably at least 56 g, 57 g, 58 g, 59 g, or 60 g of fat, or any particular amount or range comprised therein, for example, at most 5 minutes (min), 10 min, 0.25 hour (h), 0.5 h, 1 h, 2 h, 3 h, 4 h, 5 h, 6 h, 7 h, 8 h, 9 h, 10 h, 11 h or 12 h, preferably 0 min to 2 h or 5 min to 2 h, more preferably 0.25 h to 1 h, prior to the administration of a compound of formula (I). In some embodiments, the term “fed state” means that a subject, preferably a human subject, who has ingested at least 10 kcal, preferably at least 20 kcal, preferably at least 30 kcal, preferably at least 40 kcal, preferably at least 50 kcal, preferably at least 100 kcal, preferably at least 150 kcal, preferably at least 200 kcal, preferably at least 250 kcal, preferably at least 300 kcal, preferably at least 350 kcal, preferably at least 400 kcal, preferably at least 450 kcal, preferably at least 500 kcal or more, or any-sub range therein, and at least 0.1 g of fat, preferably at least 0.2 g, preferably at least 0.3 g, preferably at least 0.4 g, preferably at least 0.5 g, preferably at least 0.6 g, preferably at least 0.7 g, preferably at least 0.8 g, preferably at least 0.9 g of fat, preferably at least 1 g of fat, preferably at least 2 g, preferably at least 3 g, preferably at least 4 g, preferably at least 5 g of fat, preferably at least 6 g of fat, preferably at least 7 g, preferably at least 8 g, preferably at least 9 g, preferably at least 10 g of fat, preferably at least 11 g of fat, preferably at least 12 g, preferably at least 13 g, preferably at least 14 g, preferably at least 15 g of fat; preferably at least 16 g of fat, preferably at least 17 g, preferably at least 18 g, preferably at least 19 g, preferably at least 20 g of fat; preferably at least 21 g of fat, preferably at least 22 g, preferably at least 23 g, preferably at least 24 g, preferably at least 25 g of fat; preferably at least 26 g of fat, preferably at least 27 g, preferably at least 28 g, preferably at least 29 g, preferably at least 30 g of fat; preferably at least 31 g of fat, preferably at least 32 g, preferably at least 33 g, preferably at least 34 g, preferably at least 35 g of fat; preferably at least 36 g, preferably at least 37 g, preferably at least 38 g, preferably at least 39 g, preferably at least 40 g of fat; preferably at least 41 g, preferably at least 42 g, preferably at least 43 g, preferably at least 44 g, preferably at least 45 g of fat; preferably at least 46 g, preferably at least 47 g, preferably at least 48 g, preferably at least 49 g, preferably at least 50 g of fat; preferably at least 51 g, preferably at least 52 g, preferably at least 53 g, preferably at least 54 g, preferably at least 55 g of fat; preferably at least 56 g, preferably at least 57 g, preferably at least 58 g, preferably at least 59 g, preferably at least 60 g of fat, or any particular amount or range comprised therein, for example, at most 5 minutes (min), preferably at most 10 min, preferably at most 0.25 hour (h), preferably at most 0.5 h, preferably at most 1 h, preferably at most 2 h, preferably at most 3 h, preferably at most 4 h, preferably at most 5 h, preferably at most 6 h, preferably at most 7 h, preferably at most 8 h, preferably at most 9 h, preferably at most 10 h, preferably at most 11 h, preferably at most 12 h, preferably from at least 0 min to at most 2 h, preferably from at least 5 min to at most 2 h, more preferably from at least 0.25 h to at most 1 h, prior to the administration of a compound of formula (I). In some embodiments, the term “fed state” means that a subject, preferably a human subject, who has ingested at least 10, 20, 30, 40, 50, 100, 150, 200, 250, 300, 350, 400, 450, or 500 kcal or more, or any-sub range therein, and at least 0.1 g, 0.2 g, 0.3 g, 0.4 g, 0.5 g, 0.6 g, 0.7 g, 0.8 g or 0.9 g of fat, preferably at least 1 g, 2 g, 3 g, 4 g or 5 g of fat, preferably at least 6 g, 7 g, 8 g, 9 g or 10 g of fat, preferably at least 11 g, 12 g, 13 g, 14 g or 15 g of fat; preferably at least 16 g, 17 g, 18 g, 18 g, 19 g or 20 g of fat; preferably at least 21 g, 22 g, 23 g, 24 g or 25 g of fat; preferably at least 26 g, 27 g, 28 g, 29 g or 30 g of fat; preferably at least 31 g, 32 g, 33 g, 34 g, or 35 g of fat; preferably at least 36 g, 37 g, 38 g, 39 g, or 40 g of fat; preferably at least 41 g, 42 g, 43 g, 44 g, or 45 g of fat; preferably at least 46 g, 47 g, 48 g, 49 g, or 50 g of fat; preferably at least 51 g, 52 g, 53 g, 54 g, or 55 g of fat; preferably at least 56 g, 57 g, 58 g, 59 g, or 60 g of fat, or any particular amount or range comprised therein, for example, at most 5 minutes (min), 10 min, 0.25 hour (h), 0.5 h, 1 h, 2 h, 3 h, 4 h, 5 h, 6 h, 7 h, 8 h, 9 h, 10 h, 11 h or 12 h, preferably 0 min to 2 h or 5 min to 2 h, more preferably 0.25 h to 1 h, prior to the administration of a compound of formula (I). In some embodiments, the term “fed state” means that a subject, preferably a human subject, who has ingested at least 10 kcal, preferably at least 20 kcal, preferably at least 30 kcal, preferably at least 40 kcal, preferably at least 50 kcal, preferably at least 100 kcal, preferably at least 150 kcal, preferably at least 200 kcal, preferably at least 250 kcal, preferably at least 300 kcal, preferably at least 350 kcal, preferably at least 400 kcal, preferably at least 450 kcal, preferably at least 500 kcal or more, or any-sub range therein, and at least 21 g of fat, preferably at least 22 g, preferably at least 23 g, preferably at least 24 g, preferably at least 25 g of fat; preferably at least 26 g, preferably at least 27 g, preferably at least 28 g, preferably at least 29 g, preferably at least 30 g of fat; preferably at least 31 g, preferably at least 32 g, preferably at least 33 g, preferably at least 34 g, preferably at least 35 g of fat; preferably at least 36 g, preferably at least 37 g, preferably at least 38 g, preferably at least 39 g, preferably at least 40 g of fat; preferably at least 41 g, preferably at least 42 g, preferably at least 43 g, preferably at least 44 g, preferably at least 45 g of fat; preferably at least 46 g, preferably at least 47 g, preferably at least 48 g, preferably at least 49 g, preferably at least 50 g of fat; preferably at least 51 g, preferably at least 52 g, preferably at least 53 g, preferably at least 54 g, preferably at least 55 g of fat; preferably at least 56 g, preferably at least 57 g, preferably at least 58 g, preferably at least 59 g, preferably at least 60 g of fat, or any particular amount or range comprised therein, for example, at most 5 minutes (min), preferably at most 10 min, preferably at most 0.25 hour (h), preferably at most 0.5 h, preferably at most 1 h, preferably at most 2 h, preferably at most 3 h, preferably at most 4 h, preferably at most 5 h, preferably at most 6 h, preferably at most 7 h, preferably at most 8 h, preferably at most 9 h, preferably at most 10 h, preferably at most 11 h, preferably at most 12 h, preferably from at least 0 min to at most 2 h, preferably from at least 5 min to at most 2 h, more preferably from at least 0.25 h to at most 1 h, prior to the administration of a compound of formula (I). In some embodiments, the term “fed state” means that a subject, preferably a human subject, who has ingested at least 10, 20, 30, 40, 50, 100, 150, 200, 250, 300, 350, 400, 450, or 500 kcal or more, or any-sub range therein, and at least 21 g, 22 g, 23 g, 24 g or 25 g of fat; preferably at least 26 g, 27 g, 28 g, 29 g or 30 g of fat; preferably at least 31 g, 32 g, 33 g, 34 g, or 35 g of fat; preferably at least 36 g, 37 g, 38 g, 39 g, or 40 g of fat; preferably at least 41 g, 42 g, 43 g, 44 g, or 45 g of fat; preferably at least 46 g, 47 g, 48 g, 49 g, or 50 g of fat; preferably at least 51 g, 52 g, 53 g, 54 g, or 55 g of fat; preferably at least 56 g, 57 g, 58 g, 59 g, or 60 g of fat, or any particular amount or range comprised therein, for example, at most 5 minutes (min), 10 min, 0.25 hour (h), 0.5 h, 1 h, 2 h, 3 h, 4 h, 5 h, 6 h, 7 h, 8 h, 9 h, 10 h, 11 h or 12 h, preferably 0 min to 2 h or 5 min to 2 h, more preferably 0.25 h to 1 h, prior to the administration of a compound of formula (I).

As used herein, a “standardized meal” (standardized breakfast or standardized dinner) comprises approximately 21 g fat, 67 g carbohydrates, 19 g proteins resulting in around 533 kcal. Preferably a standardized meal comprises at least 15 g fat to at most 40 g fat; preferably at least 16 g fat to at most 38 g fat; preferably at least 17 g fat to at most 35 g fat; preferably at least 17 g fat to at most 33 g fat; preferably at least 18 g fat to at most 31 g of fat; preferably at least 18 g of fat to at most 30 g of fat; preferably at least 19 g of fat to at most 27 g of fat.

A “high-fat, high-calorie meal” (high-fat, high-calorie breakfast or high-fat, high-calorie dinner) comprises approximately: 500-600 kcal fat (56-67 g fat), 250 kcal carbohydrates, 150 kcal proteins; total calories 900-1000 kcal. Preferably a high-fat, high-calorie meal comprises at least 35 g fat to at most 80 g fat; preferably at least 40 g fat to at most 75 g fat; preferably at least 45 g fat to at most 70 g fat; preferably at least 50 to at most 75 g fat; preferably at least 50 g fat to at most 70 g of fat, preferably 50 g to at most 60 g of fat.

A “low-fat, low-calorie meal” (low-fat, low-calorie breakfast or low-fat, low-calorie dinner) comprises approximately 2.5 g fat; 247 kcal or approximately 9 g fat; 330 kcal. Preferably a low-fat, low-calorie meal comprises at least 0.1 g fat to at most 15 g fat; preferably at least 0.3 g fat to at most 13 g fat; preferably at least 0.5 g fat to at most 10 g fat; preferably at least 0.8 g fat to at most 9 g fat; preferably at least 0.9 g fat to at most 8 g of fat; preferably at least 1.0 g of fat to at most 8 g of fat; preferably at least 1.2 g of fat to at most 7 g of fat.

A “low-fat, high-calorie meal” (low-fat, high-calorie breakfast or low-fat, high-calorie dinner) comprises approximately 4.2 g fat; 911 kcal or approximately 0.95 g fat; 406 kcal. Preferably a low-fat, low-calorie meal comprises at least 0.1 g fat to at most 15 g fat; preferably at least 0.3 g fat to at most 13 g fat; preferably at least 0.5 g fat to at most 10 g fat; preferably at least 0.8 g fat to at most 9 g fat; preferably at least 0.9 g fat to at most 8 g of fat; preferably at least 1.0 g of fat to at most 8 g of fat; preferably at least 1.2 g of fat to at most 7 g of fat.

A “standardized normal fat meal” (standardized normal fat breakfast or standardized normal fat dinner) comprises approximately 21 g of fat, 67 g of carbohydrates, 19 g of proteins resulting in around 533 kcal. An example of a standardized normal fat meal would be 4 slices of bread, 2 slices of ham or cheese, butter, jelly, and 1 or 2 cups (up to 355 mL) of decaffeinated coffee or tea with milk and/or sugar. Preferably a standardized normal fat comprises at least 15 g fat to at most 40 g fat; preferably at least 16 g fat to at most 38 g fat; preferably at least 17 g fat to at most 35 g fat; preferably at least 17 g fat to at most 33 g fat; preferably at least 18 g fat to at most 31 g of fat; preferably at least 18 g of fat to at most 30 g of fat; preferably at least 19 g of fat to at most 27 g of fat. As used herein, the term “oral dosage form” refers to a pharmaceutical formulation that contains a specified amount (dose) of a compound of formula (I) as the active ingredient, in particular Compound (a), or a stereoisomeric form, a pharmaceutically acceptable salt and/or solvate and/or polymorph thereof, and inactive components (pharmaceutically acceptable excipients), formulated into a particular configuration that is suitable for oral administration and drug delivery, such as a tablet, capsule or liquid oral formulation. In one embodiment, the compositions are in the form of a tablet that can be scored.

As used herein, the term “administration” refers to introducing an agent or an active ingredient, such as a compound of formula (I) into a subject, preferably a human subject. The related terms “administering” and “administration of” (and grammatical equivalents) refer both to direct administration, which may be administration to a subject by a medical professional or by self-administration by the subject, and/or to indirect administration, which may be the act of prescribing a drug. For example, a physician who instructs a patient to self-administer a drug and/or provides a patient with a prescription for a drug.

For use in medicine, cocrystals or salts of compounds of formula (I), in particular of Compound (a), as disclosed herein refer to non-toxic “pharmaceutically acceptable salts.” “Pharmaceutically acceptable” may mean approved or approvable by a regulatory agency of the Federal or a state government or the corresponding agency in countries other than the United States, or that is listed in the U. S. Pharmacopoeia or other generally recognized pharmacopoeia for use in animals, and more particularly, in humans.

Other salts may, however, be useful in the preparation of compounds of formula (I), in particular of Compound (a), or of their pharmaceutically acceptable salt forms thereof. Suitable pharmaceutically acceptable salts of compounds of Formula (I), in particular of Compound (a), include acid addition salts that can, for example, be formed by mixing a solution of the compound with a solution of a pharmaceutically acceptable acid such as, hydrochloric acid, sulfuric acid, fumaric acid, maleic acid, succinic acid, acetic acid, benzoic acid, citric acid, tartaric acid, carbonic acid or phosphoric acid. Furthermore, where the compounds of Formula (I), in particular Compound (a), carry an acidic moiety, suitable pharmaceutically acceptable salts thereof may include alkali metal salts such as, sodium or potassium salts; alkaline earth metal salts such as, calcium or magnesium salts; and salts formed with suitable organic ligands such as, quaternary ammonium salts. Thus, representative pharmaceutically acceptable salts include acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide, valerate, and combinations of one or more thereof.

Representative acids and bases that may be used in the preparation of pharmaceutically acceptable salts include acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (+)-DL-lactic acid, lactobionic acid, maleic acid, (−)-L-malic acid, malonic acid, (+)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebaic acid, stearic acid, succinic acid, sulfuric acid, tannic acid, (+)-L-tartaric acid, thiocyanic acid, p-toluenesulfonic acid and undecylenic acid; and bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, sodium hydroxide, triethanolamine, tromethamine, zinc hydroxide, and combinations of one or more thereof.

Where the compounds of formula (I), in particular Compound (a), have at least one chiral center, they may accordingly exist as enantiomers. Where the compounds possess two or more chiral centers, they may additionally exist as diastereomers. It is to be understood that all such isomers and mixtures thereof are encompassed within the scope of the present invention. Furthermore, some of the compounds may exist as polymorphs and as such are intended to be included in the present invention. In addition, some of the compounds may form solvates with water (i.e., hydrates) or organic solvents, and such solvates are also intended to be encompassed within the scope of this invention. The skilled artisan will understand that the term compound as used herein, is meant to include solvated compounds of formula (I) (in particular solvates of Compound (a)), hydrated compounds of formula (I) (in particular hydrates of Compound (a)), and hydrated and solvated compounds of formula (I) (e.g., a compound of formula (I) in a solvate form with water and an organic solvent, in particular Compound (a) in a solvate form with water and an organic solvent).

Where the processes for the preparation of the compounds of formula (I), in particular of Compound (a), give rise to mixture of stereoisomers, these isomers may be separated by conventional techniques such as, preparative chromatography. The compounds may be prepared in racemic form, or individual enantiomers may be prepared either by enantiospecific synthesis or by resolution. The compounds may, for example, be resolved into their component enantiomers by standard techniques such as, the formation of diastereomeric pairs by salt formation with an optically active acid such as, (−)-di-p-toluoyl-d-tartaric acid and/or (+)-di-p-toluoyl-1-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral stationary phase or iso chiral column.

In one embodiment of the pharmaceutical formulation of the present invention, the compound of formula (I) is a compound comprising, consisting of, and/or consisting essentially of the (+)-enantiomer wherein said compound is substantially free from the (−)-isomer. In the present context, substantially free means less than about 25%, preferably less than about 10%, more preferably less than about 5%, even more preferably less than about 2% and even more preferably less than about 1% of the (−)-isomer calculated as

$$\% ( + ) - enantiomer = \frac{( mass ( + ) - enantiomer )}{( m a s s ( + ) - e n a n t i o m e r ) + ( m a s s ( - ) - e n a n t i o m e r )} \times 100 .$$

In another embodiment of the pharmaceutical formulation of the present invention, the compound of formula (I) is a compound comprising, consisting of, and consisting essentially of the (−)-enantiomer wherein said compound is substantially free from the (+)-isomer. In the present context, substantially free from means less than about 25%, preferably less than about 10%, more preferably less than about 5%, even more preferably less than about 2% and even more preferably less than about 1% of the (+)-isomer calculated as

$$\% ( - ) - enantiomer = \frac{( m a s s ( - ) - e n a n t i o m e r )}{( m a s s ( + ) - e n a n t i o m e r ) + ( m a s s ( - ) - e n a n t i o m e r )} \times 100.$$

During any of the processes for preparation of the compounds of the various embodiments of the present invention, it may be necessary and/or desirable to protect sensitive or reactive groups on any of the molecules concerned. This may be achieved by means of conventional protecting groups such as those described in Protective Groups in Organic Chemistry, Second Edition, J. F. W. McOmie, Plenum Press, 1973; T. W. Greene & P. G. M. Wuts, Protective Groups in Organic Synthesis, John Wiley & Sons, 1991; and T. W. Greene & P. G. M. Wuts, Protective Groups in Organic Synthesis, Third Edition, John Wiley & Sons, 1999. The protecting groups may be removed at a convenient subsequent stage using methods known from the art.

The compounds of Formula (I), in particular Compound (a), may exist in unsolvated as well as solvated forms such as, for example, hydrates, organic solvent solvates, or a combination of an organic solvent solvate and a hydrate.

As used herein, the term “solvate” means a solvent addition form that contains either a stoichiometric or non-stoichiometric amounts of one or more solvent(s). Some compounds have a tendency to trap a fixed molar ratio of solvent molecules in the crystalline solid state, thus forming a solvate. If the solvent is water, the solvate formed is a hydrate, when the solvent is alcohol, the solvate formed is an alcoholate. In certain embodiments, the solvate formed may be a combination of an organic solvent solvate and a hydrate. Hydrates are formed by the combination of one or more molecules of water with one of the substances in which the water retains its molecular state as H₂O, such combination being able to form one or more hydrate. In the hydrates, the water molecules are attached through secondary valencies by intermolecular forces, in particular hydrogen bridges. Solid hydrates contain water as so-called crystal water in stoichiometric ratios, where the water molecules do not have to be equivalent with respect to their binding state. Examples of hydrates are sesquihydrates, monohydrates, dihydrates or trihydrates. Equally suitable are the hydrates of salts of the compounds used herein.

When a compound is crystallized from a solution or slurry, it can be crystallized in a different arrangement lattice of spaces (this property is called “polymorphism”) to form crystals with different crystalline forms, each of which is known as “polymorphs”. “Polymorph”, as used herein, refers to a crystal form of a compound of Formula (I), where the molecules are localized in the three-dimensional lattice sites. Different polymorphs of the compound of Formula (I) may be different from each other in one or more physical properties, such as solubility and dissolution rate, true specific gravity, crystal form, accumulation mode, flowability and/or solid state stability. Etc.

The compounds of formula (I) may be administered as crystalline or amorphous products. They may be obtained for example as solid plugs, powders, or films by methods such as precipitation, crystallization, freeze drying, spray drying, or evaporative drying. They may be administered alone or in combination with one or more other compounds described herein or in combination with one or more other drugs. Generally, they will be administered as a formulation in association with one or more pharmaceutically acceptable excipients. The choice of pharmaceutically acceptable excipient depends largely on factors such as the particular mode of administration, the effect of the excipient on solubility and stability, and the nature of the dosage form.

Unless stated otherwise, the terms “wt %” and “weight percent” are used interchangeably to refer to the concentration of an ingredient (e.g., pharmaceutically acceptable excipient or active pharmaceutical ingredient) by weight of the pharmaceutical formulation.

An average molecular weight may, for example, refer to a number average or weight average molecular weight. Average molecular weight may, for example, be measured using gel permeation chromatography.

The term “subject” refers to an animal, preferably a mammal, most preferably a human, who has been the object of treatment, observation or experiment. In one embodiment, the subject is a human. In some embodiments, the subject is a human who has been diagnosed with a condition, or a disorder or a disease caused by a Dengue virus or with a dengue viral infection. In one embodiment, the subject is a human who has been diagnosed with a condition, or a syndrome, or a disorder or a disease caused by a Dengue virus. In some embodiments, the subject is a human who has not been diagnosed with a condition, or a disorder or a disease caused by a Dengue virus or a dengue viral infection and who will be taking the treatment preventively. In one embodiment, the subject is a human who has not been diagnosed with a condition, or a syndrome, or a disorder or a disease caused by a Dengue virus and who will be taking the treatment preventively.

The term “therapeutically effective amount” refers to an amount of an active compound or pharmaceutical agent which elicits the biological or medicinal response in a subject, a biological sample, a tissue system, animal or human that is being sought by a researcher, veterinarian, medical doctor or other clinician, including reduction or inhibition of an enzyme or a protein activity, or ameliorating symptoms, alleviating conditions, slowing or delaying disease progression, or preventing a disease.

Preferably, the term “therapeutically effective amount” may refer to administering a dosing a regimen to a subject, preferably a human subject, to achieve a certain plasma concentration level or exposure of the active compound or pharmaceutical agent that would result in efficacious levels for the treatment or prevention of dengue virus or a dengue viral infection in the subject.

Preferably, the term “therapeutically effective amount” may refer to the amount of a compound, a formulation, or a dosage form that, when administered to a subject, preferably a human subject, is effective to at least partially alleviate, inhibit, prevent, treat, and/or ameliorate a condition, or a disorder, or a symptom, or a disease caused by a Dengue virus.

As used herein, the term “dengue viral replication inhibitor” refers to an agent that inhibits or reduces at least one condition, symptom, syndrome, disorder, and/or disease caused by a Dengue virus.

As used herein, unless otherwise noted, the term “affect” or “affected” (when referring to a disease, syndrome, condition or disorder that is affected by the inhibition of a Dengue virus replication) includes a reduction in the frequency and/or severity of one or more symptoms or manifestations of said disease, syndrome, condition or disorder; and/or includes the prevention of the development of one or more symptoms or manifestations of said disease, syndrome, condition or disorder or the development of the disease, condition, syndrome or disorder.

As used herein, the term “Cₘₐₓ”, refers to the observed maximum (peak) plasma concentration of a specified compound in a subject after administration of a dose of that compound to the subject.

As used herein, the term “AUC”, refers to the area under the concentration-time curve, which is a measure of exposure to a compound of interest, and is the integral of the concentration-time curve after a single dose or at steady state. AUC is expressed in units of μg*hr/L (μg×hr/L).

As used herein, the term “adverse event” (AE) refers to any untoward medical occurrence in a subject, preferably a human subject, administered a pharmaceutical formulation according to the invention. An AE does not necessarily have a causal relationship with the administration of the pharmaceutical formulation according to the invention. An AE can therefore be any unfavorable and unintended sign (including an abnormal finding), symptom, or disease temporally associated with the use of a pharmaceutical formulation, that is directly related to that pharmaceutical formulation. This includes any occurrence that is new in onset or aggravated in severity or frequency from the baseline condition, or abnormal results of diagnostic procedures, including laboratory test abnormalities.

As used herein, the term “treatment-emergent adverse event” refers to any adverse event which has a causal relationship with the administration of the pharmaceutical formulation according to the invention.

As used herein, an “pharmaceutically acceptable excipient” is an inactive ingredient in a pharmaceutical formulation. Examples of excipients include diluents, wetting agents (e.g., surfactants), binders, glidants, lubricants, disintegrants, fillers, surfactants and the like.

As used herein, a “disintegrant agent” or “disintegrant” is an excipient that hydrates a pharmaceutical formulation and aids in tablet dispersion. Examples of disintegrant agents include croscarmellose 5 sodium, crospovidone (i.e., cross-linked polyvinyl N-pyrrolidone), sodium starch glycolate, or any combination thereof.

As used herein, a “diluent” or “filler” is an excipient that adds bulkiness to a pharmaceutical formulation. Examples of diluents include lactose, sorbitol, celluloses, calcium phosphates, starches, sugars (e.g., mannitol, sucrose, or the like) or any combination thereof.

As used herein, a “wetting agent” or a “surfactant” is an excipient that imparts pharmaceutical formulations with enhanced solubility and/or wettability. Examples of wetting agents include sodium lauryl sulfate (SLS), sodium stearyl fumarate (SSF), polyoxyethylene 20 sorbitan mono-oleate (i.e. polysorbate 20) (e.g., Tween™ or Tween 20), Soluplus®, or any combination thereof.

As used herein, a “binder” is an excipient that imparts a pharmaceutical formulation with enhanced cohesion or tensile strength (e.g., hardness). Examples of binders include dibasic calcium phosphate, sucrose, corn (maize) starch, microcrystalline cellulose, and modified cellulose (e.g., hydroxymethyl cellulose).

As used herein, a “glidant” is an excipient that imparts pharmaceutical formulation with enhanced flow properties. Examples of glidants include colloidal silica and/or talc.

As used herein, a “colorant” is an excipient that imparts a pharmaceutical formulation with a desired color. Examples of colorants include commercially available pigments such as FD&C Blue #1 Aluminum Lake, FD&C Blue #2, other FD&C Blue colors, titanium dioxide, iron oxide, and/or combinations thereof. Other colorants include commercially available pigments such as FD&C Green #3.

As used herein, a “lubricant” is an excipient that is added to pharmaceutical formulation that are pressed into tablets. The lubricant aids in compaction of granules into tablets and ejection of a tablet of a pharmaceutical formulation from a die press. Examples of lubricants include magnesium stearate, stearic acid (stearin), hydrogenated oil, sodium stearyl fumarate, or any combination thereof.

Preferred statements (features) and embodiments of the pharmaceutical formulations, uses and process of this invention are set herein below. Each statement and embodiment of the invention so defined may be combined with any other statement and/or embodiment unless clearly indicated to the contrary. In particular, any feature indicated as being preferred or advantageous may be combined with any other feature or features or statement indicated as being preferred or advantageous. Hereto, the present invention is in particular captured by any one or any combination of one or more of the below numbered aspects and embodiments, with any other statement and/or embodiment.
1. A compound of formula (I) for use in the prevention and/or treatment of dengue viral infections, wherein the compound of formula (I) is administered in an oral dosage form to a human subject in either a fed or fasted state and wherein compound of formula (I) corresponds to

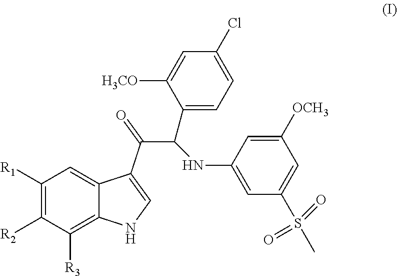

a stereo-isomeric form, a pharmaceutically acceptable salt, solvate or polymorph thereof;
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃.
2. The compound for use according to statement 1, wherein the compound is administered in an oral dosage form to a human subject in a fed state; preferably the human subject in a fed state has eaten a standard normal fat meal, a high fat-high calorie meal, a low fat-low calorie meal, or a low fat-high calorie meal.
3. The compound for use according to any one of statements 1 or 2, wherein said human subject is infected with Dengue virus or at risk of being infected with Dengue virus.
4. The compound for use according to any one of statement 1 to 3, wherein said human subject is in a fed state before or simultaneously with the administration of said oral dosage form.
5. The compound for use according to any one of statements 1 to 4, wherein the human is in a fed state and has eaten less than 4 hours, preferably less than 3 hours, preferably less than 2 hours, preferably less than 1 hour, preferably less than 30 minutes, preferably less than 15 minutes, preferably less than 10 minutes before the time of administering the oral dosage form, preferably the oral dosage form is administered at most within 30 minutes of eating; preferably at most within 25 minutes of eating; preferably at most within 20 minutes of eating; preferably at most within 15 minutes of eating; preferably at most within 10 minutes of eating; preferably at most within 5 minutes of eating; preferably simultaneously with eating.
6. The compound for use according to any one of statements 1 to 4, wherein the oral dosage form is administered in the morning. Said oral dosage form may be administered simultaneously or after the first meal, the second meal or the third meal of the day.
7. The compound for use according to any one of statements 1 to 5, wherein said human in a fed state has eaten food comprising at least 0.1 g of fat, preferably at least 0.2 g, preferably at least 0.3 g, preferably at least 0.4 g, preferably at least 0.5 g, preferably at least 0.6 g, preferably at least 0.7 g, preferably at least 0.8 g, preferably at least 0.9 g of fat, preferably at least 1 g of fat, preferably at least 2 g, preferably at least 3 g, preferably at least 4 g, preferably at least 5 g of fat, preferably at least 6 g of fat, preferably at least 7 g, preferably at least 8 g, preferably at least 9 g, preferably at least 10 g of fat, preferably at least 11 g of fat, preferably at least 12 g, preferably at least 13 g, preferably at least 14 g, preferably at least 15 g of fat; preferably at least 16 g of fat, preferably at least 17 g, preferably at least 18 g, preferably at least 19 g, preferably at least 20 g of fat; preferably at least 21 g of fat, preferably at least 22 g, preferably at least 23 g, preferably at least 24 g, preferably at least 25 g of fat; preferably at least 26 g of fat, preferably at least 27 g, preferably at least 28 g, preferably at least 29 g, preferably at least 30 g of fat; preferably at least 31 g of fat, preferably at least 32 g, preferably at least 33 g, preferably at least 34 g, preferably at least 35 g of fat; preferably at least 36 g of fat, preferably at least 37 g, preferably at least 38 g, preferably at least 39 g, preferably at least 40 g of fat; preferably at least 41 g of fat, preferably at least 42 g, preferably at least 43 g, preferably at least 44 g, preferably at least 45 g of fat; preferably at least 46 g of fat, preferably at least 47 g, preferably at least 48 g, preferably at least 49 g, preferably at least 50 g of fat; preferably at least 51 g of fat, 52 g, preferably at least 53 g, preferably at least 54 g, preferably at least 55 g of fat; preferably at least 56 g of fat, preferably at least 57 g, preferably at least 58 g, preferably at least 59 g, preferably at least 60 g of fat, or any particular amount or range comprised therein.
8. The compound for use according to any one of statements 1 to 7, wherein said human in a fed state has eaten food comprising at least 0.1 g of fat, preferably at least 0.2 g, preferably at least 0.3 g, preferably at least 0.4 g, preferably at least 0.5 g, preferably at least 0.6 g, preferably at least 0.7 g, preferably at least 0.8 g, preferably at least 0.9 g of fat, preferably at least 1 g of fat, preferably at least 2 g, preferably at least 3 g, preferably at least 4 g, preferably at least 5 g of fat, preferably at least 6 g of fat, preferably at least 7 g, preferably at least 8 g, preferably at least 9 g, preferably at least 10 g of fat, preferably at least 11 g of fat, preferably at least 12 g, preferably at least 13 g, preferably at least 14 g, preferably at least 15 g of fat; preferably at least 16 g of fat, preferably at least 17 g, preferably at least 18 g, preferably at least 19 g, preferably at least 20 g of fat; preferably at least 21 g of fat, preferably at least 22 g, preferably at least 23 g, preferably at least 24 g, preferably at least 25 g of fat; preferably at least 26 g of fat, preferably at least 27 g, preferably at least 28 g, preferably at least 29 g, preferably at least 30 g of fat; preferably at least 31 g of fat, preferably at least 32 g, preferably at least 33 g, preferably at least 34 g, preferably at least 35 g of fat; preferably at least 36 g of fat, preferably at least 37 g, preferably at least 38 g, preferably at least 39 g, preferably at least 40 g of fat; preferably at least 41 g of fat, preferably at least 42 g, preferably at least 43 g, preferably at least 44 g, preferably at least 45 g of fat; preferably at least 46 g of fat, preferably at least 47 g, preferably at least 48 g, preferably at least 49 g, preferably at least 50 g of fat; preferably at least 51 g of fat, 52 g, preferably at least 53 g, preferably at least 54 g, preferably at least 55 g of fat; preferably at least 56 g of fat, preferably at least 57 g, preferably at least 58 g, preferably at least 59 g, preferably at least 60 g of fat, or any particular amount or range comprised therein;
   and wherein the human has eaten less than 4 hours from administration of the compound of formula I, preferably less than 3 hours, preferably less than 2 hours, preferably less than 1 hour, preferably less than 30 minutes, preferably less than 15 minutes, preferably less than 10 minutes before the time of administering the oral dosage form, preferably the oral dosage form is administered at most within 30 minutes of eating; preferably at most within 25 minutes of eating; preferably at most within 20 minutes of eating; preferably at most within 15 minutes of eating; preferably at most within 10 minutes of eating; preferably at most within 5 minutes of eating from administration of the compound of formula I, such as Compound (a); preferably the compound of formula I, such as Compound (a), is administered simultaneously with eating.
9. The compound for use according to any one of statements 1 to 8, wherein said human in a fed state has eaten food comprising at least 0.1 g, 0.2 g, 0.3 g, 0.4 g, 0.5 g, 0.6 g, 0.7 g, 0.8 g or 0.9 g of fat, preferably at least 1 g, 2 g, 3 g, 4 g or 5 g of fat, preferably at least 6 g, 7 g, 8 g, 9 g or 10 g of fat, preferably at least 11 g, 12 g, 13 g, 14 g or 15 g of fat; preferably at least 16 g, 17 g, 18 g, 18 g, 19 g or 20 g of fat; preferably at least 21 g, 22 g, 23 g, 24 g or 25 g of fat; preferably at least 26 g, 27 g, 28 g, 29 g or 30 g of fat; preferably at least 31 g, 32 g, 33 g, 34 g, or 35 g of fat; preferably at least 36 g, 37 g, 38 g, 39 g, or 40 g of fat; preferably at least 41 g, 42 g, 43 g, 44 g, or 45 g of fat; preferably at least 46 g, 47 g, 48 g, 49 g, or 50 g of fat; preferably at least 51 g, 52 g, 53 g, 54 g, or 55 g of fat; preferably at least 56 g, 57 g, 58 g, 59 g, or 60 g of fat, or any particular amount or range comprised therein.
10. The compound for use according to any one of statements 1 to 9, wherein said human in a fed state has eaten food having a total energy count of at least 10 kcal, preferably at least 20 kcal, preferably at least 30 kcal, preferably at least 40 kcal, preferably at least 50 kcal, preferably at least 60 kcal, preferably at least 70 kcal, preferably at least 80 kcal, preferably at least 90 kcal, preferably at least 100 kcal, preferably at least 110 kcal, preferably at least 120 kcal, preferably at least 130 kcal, preferably at least 140 kcal, preferably at least 150 kcal, preferably at least 160 kcal, preferably at least 170 kcal, preferably at least 180 kcal, preferably at least 190 kcal, preferably at least 200 kcal, preferably at least 210 kcal, preferably at least 220 kcal, preferably at least 230 kcal, preferably at least 240 kcal, preferably at least 250 kcal, preferably at least 260 kcal, preferably at least 270 kcal, preferably at least 280 kcal, preferably at least 290 kcal, preferably at least 300 kcal, preferably at least 310 kcal, preferably at least 320 kcal, preferably at least 330 kcal, preferably at least 340 kcal, preferably at least 350 kcal, preferably at least 360 kcal, preferably at least 370 kcal, preferably at least 380 kcal, preferably at least 390 kcal, preferably at least 400 kcal, preferably at least 410 kcal, preferably at least 420 kcal, preferably at least 430 kcal, preferably at least 440 kcal, preferably at least 450 kcal, preferably at least 460 kcal, preferably at least 470 kcal, preferably at least 480 kcal, preferably at least 490 kcal, preferably at least 500 kcal or any particular amount or range comprised therein.
11. The compound for use according to any one of statements 1 to 10, wherein said human in a fed state has eaten food having a total energy count of at most 1500 kcal, preferably at most 1400 kcal, preferably at most 1300 kcal, preferably at most 1200 kcal, preferably at most 1110 kcal, preferably at most 1000 kcal, preferably at most 980 kcal, preferably at most 960 kcal, preferably at most 940 kcal, preferably at most 920 kcal, preferably at most 810 kcal, preferably at most 820 kcal, preferably at most 830 kcal, preferably at most 840 kcal preferably at most 850 kcal, preferably at most 860 kcal, preferably at most 870 kcal, preferably at most 880 kcal, preferably at most 890 kcal preferably at most 900 kcal, preferably at most 710 kcal, preferably at most 720 kcal, preferably at most 730 kcal, preferably at most 740 kcal, preferably at most 750 kcal, preferably at most 760 kcal, preferably at most 770 kcal, preferably at most 780 kcal, preferably at most 790 kcal preferably at most 800 kcal, preferably at most 610 kcal, preferably at most 620 kcal, preferably at most 630 kcal, preferably at most 640 kcal, preferably at most 650 kcal, preferably at most 660 kcal, preferably at most 670 kcal, preferably at most 680 kcal, preferably at most 690 kcal, preferably at most 700 kcal, preferably at most 510 kcal, preferably at most 520 kcal, preferably at most 530 kcal, preferably at most 540 kcal, preferably at most 550 kcal, preferably at most 560 kcal, preferably at most 570 kcal, preferably at most 580 kcal, preferably at most 590 kcal, preferably at most 600 kcal or any particular amount or range comprised therein.
12. The compound for use according to any one of statements 1 to 11, wherein said human in a fed state has eaten food having a total energy count of at least 10 kcal, 20 kcal, 30 kcal, 40 kcal or 50 kcal, preferably at least 60 kcal, 70 kcal, 80 kcal, 90 kcal or 100 kcal, preferably at least 110 kcal, 120 kcal, 130 kcal, 140 kcal or 150 kcal, preferably at least 160 kcal, 170 kcal, 180 kcal, 190 kcal or 200 kcal, preferably at least 210 kcal, 220 kcal, 230 kcal, 240 kcal, 250 kcal, 260 kcal, 270 kcal, 280 kcal, 290 kcal or 300 kcal, preferably at least 310 kcal, 320 kcal, 330 kcal, 340 kcal, 350 kcal, 360 kcal, 370 kcal, 380 kcal, 390 kcal or 400 kcal, preferably at least 410 kcal, 420 kcal, 430 kcal, 440 kcal, 450 kcal, 460 kcal, 470 kcal, 480 kcal, 490 kcal or 500 kcal or any particular amount or range comprised therein. Preferably said human in a fed state has eaten food having a total energy count of at most 1500 kcal, 1400 kcal, 1300 kcal, 1200 kcal or 1110 kcal, preferably at most 1000 kcal, 980 kcal, 960 kcal, 940 kcal or 920 kcal, preferably at most 810 kcal, 820 kcal, 830 kcal, 840 kcal or 850 kcal, preferably at most 860 kcal, 870 kcal, 880 kcal, 890 kcal or 900 kcal, preferably at most 710 kcal, 720 kcal, 730 kcal, 740 kcal, 750 kcal, 760 kcal, 770 kcal, 780 kcal, 790 kcal or 800 kcal, preferably at most 610 kcal, 620 kcal, 630 kcal, 640 kcal, 650 kcal, 660 kcal, 670 kcal, 680 kcal, 690 kcal or 700 kcal, preferably at most 510 kcal, 520 kcal, 530 kcal, 540 kcal, 550 kcal, 560 kcal, 570 kcal, 580 kcal, 590 kcal or 600 kcal or any particular amount or range comprised therein.
13. The compound for use according to any one of statements 1 to 12, wherein the oral dosage form is a solid dosage form formulated in a pharmaceutical formulation.
14. The compound for use according to any one of statements 1 to 13, wherein the oral dosage form, is a solid dosage form and comprises a cellulose derivative such as hydroxypropyl methylcellulose (HPMC), or methacrylic acid copolymer or a combination thereof.
15. The compound for use according to any one of statements 1 to 14, wherein the oral dosage form, is a solid dosage form comprising a cellulose derivative; preferably wherein the compound of formula (I) and said the cellulose derivative (e.g., hydroxypropyl methylcellulose) are present in the oral dosage form in a ratio of from 4:1 w/w to 1:5 w/w compound of formula (I): cellulose derivative, preferably from 3.5:1 w/w to 1:4.5 w/w compound of formula (I): cellulose derivative; preferably from 3:1 w/w to 1:4 w/w compound of formula (I): cellulose derivative; preferably from 2.5:1 w/w to 1:3.5 w/w compound of formula (I): cellulose derivative; preferably from 2:1 w/w to 1:3 w/w compound of formula (I): cellulose derivative; preferably from 2.5:1 w/w to 1:2.5 w/w compound of formula (I): cellulose derivative, preferably from 2:1 w/w to 1:2 w/w compound of formula (I): cellulose derivative; preferably from 1.5:1 w/w to 1:1.5 w/w compound of formula (I): cellulose derivative or a ratio comprised within any two ratios mentioned herein, or a ratio range or sub-range within any two ratios mentioned herein.
16. The compound for use according to any one of statements 1 to 15, wherein the oral dosage form is a solid dosage form comprising a cellulose derivative which is hydroxypropyl methylcellulose (HPMC), preferably wherein said hydroxypropyl methylcellulose has a viscosity ranging between 3 and 5000 mPa·s, in 2 wt % solution in H2O at 25° C.; preferably between 3 and 500 mPa·s, in 2 wt % solution in H2O at 25° C., preferably between 3 and 50 mPa·s in 2 wt % solution in H2O at 25° C. Preferably said cellulose derivative is hydroxypropyl methylcellulose selected from HPMC E5, HPMC E6, HPMC E15, HPMC E50, and mixtures thereof.
17. The compound for use according to any one of statements 1 to 16, wherein the oral dosage form, is a solid dosage form comprising methacrylic acid copolymer; preferably wherein the compound of formula (I) and the methacrylic acid copolymer are present in the solid dosage form in a ratio of from 4:1 w/w to 1:9 w/w, preferably from 3.9:1 w/w to 1:8 w/w; preferably from 3.8:1 w/w to 1:7 w/w; from 3.7:1 w/w to 1:6 w/w; preferably from 3:6:1 w/w to 1:5 w/w, preferably from 3.5:1 w/w to 1:4.5 w/w; preferably from 3:1 w/w to 1:4 w/w; preferably from 2.5:1 w/w to 1:3.5 w/w; preferably from 2:1 w/w to 1:3 w/w; preferably from 2.5:1 w/w to 1:2.5 w/w, preferably from 2:1 w/w to 1:2 w/w; preferably from 1.5:1 w/w to 1:1.5 w/w or a ratio comprised within any two ratios mentioned herein, or a ratio range or sub-range within any two ratios mentioned herein.
18. The compound for use according to any one of statements 1 to 17, wherein the oral dosage form is a solid dosage form comprising a copolymer selected from the group comprising a copolymer of methacrylic acid and methyl methacrylate; a copolymer of methacrylic acid and ethyl acrylate and mixtures thereof.
19. The compound for use according to any one of statements 1 to 18, wherein the oral dosage form is formulated in a pharmaceutical formulation, said formulation comprising at most 50 wt %, preferably at most 40 wt %, preferably at most 30 wt %, preferably at most 25 wt %, preferably at most 20 wt % or any particular amount or range comprised therein, of compound of formula (I) relative to the total weight of the formulation.
20. The compound for use according to any one of statements 1 to 19, wherein the solid dosage form is formulated in a pharmaceutical formulation, said formulation comprising from 0.1 wt % to 50 wt % of compound of formula (I) relative to the total weight of the formulation, preferably from 1 wt % to 40 wt. %, more preferably from 2.5 wt % to 30 wt. %, most preferably from 5 wt % to 25 wt %, or any particular amount or range comprised therein of compound of formula (I) relative to the total weight of the formulation.
21. The compound for use according to any one of statements 1 to 20, wherein the oral dosage form is formulated in a pharmaceutical formulation, said formulation further comprising one or more pharmaceutically acceptable excipients selected from the group comprising disintegrants, binders, diluents, lubricants, stabilizers, wetting agents, glidants, osmotic agents, colorants, plasticizers, coatings, fillers, surfactants, and mixtures thereof.
22. The compound for use according to any one of statements 1 to 21, wherein the oral dosage form is formulated in a pharmaceutical formulation, said formulation comprising one or more pharmaceutically acceptable excipients; wherein the formulation comprises at most 95 wt %, at most 90 wt %, at most 80 wt %, preferably at most 70 wt %, preferably at most 60 wt % of the one or more pharmaceutically acceptable excipients relative to the total weight of the formulation; and/or wherein the formulation comprises at least 10 wt %, preferably at least 20 wt %, preferably at least 30 wt % of the one or more pharmaceutically acceptable excipients relative to the total weight of the formulation.
23. The compound for use according to any one of statements 1 to 22, wherein the oral dosage form is formulated in a pharmaceutical formulation, said formulation comprising one or more diluents; preferably wherein the formulation comprises at most 95 wt %, preferably at most 80 wt %, preferably at most 75 wt % of the diluent relative to the total weight of the formulation; and/or wherein the formulation comprises at least 5 wt %, preferably at least 10 wt %, preferably at least 15 wt % of the diluent relative to the total weight of the formulation.
24. The compound for use according to any one of statements 1 to 23, wherein the oral dosage form is formulated in a pharmaceutical formulation, said formulation comprising one or more disintegrants; preferably wherein the formulation comprises at most 30 wt %, preferably at most 20 wt %, preferably at most 15 wt %, preferably at most 10 wt % of the disintegrant relative to the total weight of the formulation; and/or wherein the formulation comprises at least 1 wt %, preferably at least 2.5 wt %, preferably at least 5 wt %, preferably least 8 wt % of the disintegrant relative to the total weight of the formulation.
25. The compound for use according to any one of statements 1 to 24, wherein the oral dosage form is formulated in a pharmaceutical formulation, said formulation comprising one or more binders; preferably wherein the formulation comprises at most 50 wt %, preferably at most 45 wt %, preferably at most 40 wt %, more preferably at most 35 wt % of the binder relative to the total weight of the formulation; and/or wherein the formulation comprises at least 5 wt %, preferably at least 10 wt %, preferably at least 15 wt %, more preferably at least 20 wt %, most preferably 25 wt % of the binder relative to the total weight of the formulation.
26. The compound for use according to any one of statements 1 to 25, wherein the oral dosage form is formulated in a pharmaceutical formulation, said formulation comprising one or more lubricants; preferably wherein the formulation comprises at most 5.5 wt %, preferably at most 3.5 wt %, preferably at most 2 wt % of the lubricant relative to the total weight of the formulation; and/or wherein the formulation comprises at least 0.5 wt %, preferably at least 1 wt %, preferably at least 1.5 wt % of the lubricant relative to the total weight of the formulation.
27. The compound for use according to any one of statements 1 to 26, wherein the oral dosage form is formulated in a pharmaceutical formulation, said formulation comprising one or more wetting agent; preferably wherein the formulation comprises at most 5.5 wt %, preferably at most 3.5 wt %, preferably at most 2 wt % of the wetting agent relative to the total weight of the formulation; and/or wherein the formulation comprises at least 0.5 wt %, preferably at least 1 wt %, preferably at least 1.5 wt % of the wetting agent relative to the total weight of the formulation.
28. The compound for use according to any one of statements 1 to 27, wherein the oral dosage form is formulated in a pharmaceutical formulation, said formulation comprising one or more glidant; preferably wherein the formulation comprises at most 10 wt %, preferably at most 8 wt %, preferably at most 5 wt % of the glidant relative to the total weight of the formulation; and/or wherein the formulation comprises at least 0.1 wt %, preferably at least 1 wt %, preferably at least 2 wt % of the glidant relative to the total weight of the formulation.
29. The compound for use according to any one of statements 1 to 28, wherein the oral dosage form is formulated in a pharmaceutical formulation, said formulation comprising a plurality of granules forming an intragranular phase of the formulation and one or more pharmaceutically acceptable excipients forming an extragranular phase of the formulation.
30. The compound for use according to any one of statements 1 to 29, wherein the oral dosage form is formulated in a pharmaceutical formulation, said formulation comprising at least 15.0 wt % of intragranular phase by weight of the pharmaceutical formulation; preferably at least 20.0 wt %; preferably at least 28.0 wt %; preferably at least 34.0 wt % of intragranular phase by weight of the pharmaceutical formulation.
31. The compound for use according to any one of statements 1 to 30, wherein the oral dosage form is formulated in a pharmaceutical formulation, said formulation comprising at most 99.0 wt % of intragranular phase by weight of the pharmaceutical formulation; preferably at most 93.0 wt %; preferably at most 85.0 wt %; preferably at most 80.0 wt %; preferably at most 74.0 wt %; preferably at most 73.0 wt %; preferably at most 67.0 wt %; preferably at most 63.0 wt %; preferably at most 60.0 wt %; preferably at most 53.0 wt % of intragranular phase by weight of the pharmaceutical formulation.
32. The compound for use according to any one of statements 1 to 31, wherein the oral dosage form is formulated in a pharmaceutical formulation, said formulation comprising an intragranular phase comprising from 1.0 wt % to 70.0 wt % of compound of formula (I) by weight of the pharmaceutical formulation; preferably from 2.0 wt % to 60.0 wt %; preferably from 3.0 wt % to 55.0 wt %; preferably from 4.0 wt % to 50.0 wt %; preferably from 5.0 wt % to 45.0 wt %; preferably from 5.0 wt % to 40.0 wt %; preferably from 10.0 wt % to 35.0 wt %; preferably from 15.0 wt % to 30.0 wt % of compound of formula (I) by the total weight of the pharmaceutical formulation.
33. The compound for use according to any one of statements 1 to 32, wherein the oral dosage form is formulated in a pharmaceutical formulation, said formulation comprising (a) an active pharmaceutical ingredient (API); and (b) methacrylic acid copolymer, or a cellulose derivative such as methyl cellulose (MC), ethyl cellulose (EC), hydroxyethyl cellulose (HEC), hydroxypropyl cellulose (HPC), carboxymethyl cellulose (CMC), sodium carboxymethyl cellulose (NaCMC) or hydroxypropyl methylcellulose (HPMC), or a combination thereof. Preferably the cellulose derivative is HPMC;
   wherein said API is a compound of formula (I); preferably Compound (a).
34. The compound for use according to any one of statements 1 to 33, wherein the oral dosage form is formulated in a pharmaceutical formulation, said formulation comprising an intragranular phase comprising from 5 wt % to 60 wt %; preferably from 8 wt % to 50 wt %; preferably from 15 wt % to 45 wt % of methacrylic acid copolymer, cellulose derivative or a mixture thereof by the total weight of the pharmaceutical formulation. Preferably, the cellulose derivative is hydroxypropyl methylcellulose.
35. The compound for use according to any one of statements 1 to 34, wherein the oral dosage form is formulated in a pharmaceutical formulation, said formulation comprising an intragranular phase comprising from 5 wt % to 60 wt %; preferably from 10 wt % to 60 wt %; preferably from 10 wt % to 50 wt %; preferably from 15 wt % to 50 wt % of filler by the total weight of the pharmaceutical formulation.
36. The compound for use according to any one of statements 1 to 35, wherein the oral dosage form is formulated in a pharmaceutical formulation, said formulation comprising an intragranular phase comprising from 1 wt % to 10 wt %; preferably from 1 wt % to 9 wt %, preferably from 1.7 wt % to 8 wt % of disintegrant by the total weight of the pharmaceutical formulation.
37. The compound for use according any one of statements 1 to 36, wherein the oral dosage form is formulated in a pharmaceutical formulation, said formulation comprising an intragranular phase comprising from 0.1 wt % to 5 wt %; preferably from 0.2 wt % to 4.7 wt %; preferably from 0.5 to 4.5 wt % of glidant by the total weight of the pharmaceutical formulation.
38. The compound for use according to any one of statements 1 to 37, wherein the oral dosage form is formulated in a pharmaceutical formulation, said formulation comprising an intragranular phase comprising from 0.1 wt % to 5 wt %; preferably from 0.2 wt % to 4.7 wt %; preferably from 0.5 to 4.5 wt % of surfactant by the total weight of the pharmaceutical formulation.
39. The compound for use according to any one of statements 1 to 28, wherein the oral dosage form is formulated in a pharmaceutical formulation, said formulation comprising an intragranular phase comprising from 0.1 wt % to 3 wt %; preferably from 0.2 wt % to 2.7 wt %; preferably from 0.5 to 2.5 wt % of lubricant by the total weight of the pharmaceutical formulation.
40. The compound for use according to any one of statements 1 to 39, wherein the oral dosage form is formulated in a pharmaceutical formulation, said formulation comprising an intragranular phase comprising:
   (i) from 5 wt % to 45 wt %; preferably from 5 wt % to 40 wt %; preferably from 10 wt % to 35 wt %; preferably from 15 wt % to 30 wt % of compound of formula (I) by weight of the pharmaceutical formulation;
   (ii) from 5 wt % to 60 wt %; preferably from 8 wt % to 50 wt %; preferably from 15 wt % to 45 wt % of methacrylic acid copolymer or hydroxypropyl methylcellulose by weight of the pharmaceutical formulation; and
   optionally (iii) from 5 wt % to 60 wt %; preferably from 10 wt % to 60 wt %; preferably from 10 wt % to 50 wt %; preferably from 15 wt % to 50 wt % of filler by weight of the pharmaceutical formulation;
   optionally (iv) from 1 wt % to 10 wt %; preferably from 1 wt % to 9 wt %, preferably from 1.7 wt % to 8 wt % of disintegrant by weight of the pharmaceutical formulation;
   optionally (v) from 0.1 wt % to 50 wt %; preferably from 0.2 wt % to 4.7 wt %; preferably from 0.5 to 4.5 wt % of glidant by weight of the pharmaceutical formulation;
   optionally (vi) from 0.1 wt % to 5 wt %; preferably from 0.2 wt % to 4.7 wt %; preferably from 0.5 to 4.5 wt % of surfactant by weight of the pharmaceutical formulation; and
   optionally (vii) from 0.1 wt % to 3 wt %; preferably from 0.2 wt % to 2.7 wt %; preferably from 0.5 wt % to 2.5 wt % of lubricant by weight of the pharmaceutical formulation.
41. The compound for use according to any one of statements 1 to 40, wherein the oral dosage form is formulated in a pharmaceutical formulation, said formulation comprising at least 5 wt % of extragranular phase by weight of the pharmaceutical formulation; preferably at least 7 wt %; preferably at least 10 wt %; preferably at least 15 wt %; preferably at least 20 wt %; preferably at least 28 wt %; preferably at least 34 wt % of extragranular phase by the total weight of the pharmaceutical formulation.
42. The compound for use according to any one of statements 1 to 41, wherein the oral dosage form is formulated in a pharmaceutical formulation, said formulation comprising at most 74 wt % of extragranular phase by weight of the pharmaceutical formulation; preferably at most 73 wt %; preferably at most 67 wt %; preferably at most 63 wt %; preferably at most 53 wt. %, preferably at most 40 wt % of extragranular phase by the total weight of the pharmaceutical formulation.
43. The compound for use according to any one of statements 1 to 42, wherein the oral dosage form is formulated in a pharmaceutical formulation, said formulation comprising an extragranular phase comprising from 0.1 wt % to 5 wt % of disintegrant by weight of the pharmaceutical formulation; preferably from 0.2 wt % to 4.7 wt %; preferably from 0.5 wt % to 3.5 wt % of disintegrant by the total weight of the pharmaceutical formulation.
44. The compound for use according to any one of statements 1 to 43, wherein the oral dosage form is formulated in a pharmaceutical formulation, said formulation comprising an extragranular phase comprising from 0.1 wt % to 3 wt % of lubricant by weight of the pharmaceutical formulation; preferably from 0.2 wt % to 2.7 wt %; preferably from 0.5 wt % to 2.5 wt % of lubricant by the total weight of the pharmaceutical formulation.
45. The compound for use according to any one of statements 1 to 44, wherein the oral dosage form is formulated in a pharmaceutical formulation, said formulation comprising an extragranular phase comprising from 0.1 wt % to 55 wt % of filler by weight of the pharmaceutical formulation; preferably from 0.2 wt % to 50 wt %; preferably from 0.3 wt % to 45 wt %; preferably from 0.4 wt % to 40 wt %; preferably from 0.5 wt % to 35 wt %; preferably from 0.6 wt % to 30 wt %; preferably from 0.7 to 25 wt %; preferably from 0.8 wt % to 20 wt %; preferably from 1 wt % to 15 wt %; preferably from 1.3 wt % to 12 wt %; preferably from 2 wt % to 10 wt %; preferably from 2.5 wt % to 9 wt %; preferably from 3 wt % to 8 wt %; preferably from 4 wt % to 7 wt %; preferably from 5 wt % to 6 wt % of filler by the total weight of the pharmaceutical formulation.
46. The compound for use according to any one of statements 1 to 45, wherein the oral dosage form is formulated in a pharmaceutical formulation, said formulation comprising an extragranular phase comprising:
   (a) from 0.1 wt % to 5 wt % of disintegrant by weight of the pharmaceutical formulation; preferably from 0.2 wt % to 4.7 wt %; preferably from 0.5 wt % to 3.5 wt %; more preferably from 1 wt % to 3 wt % of disintegrant by the total weight of the pharmaceutical formulation;
   (b) from 0.1 wt % to 3.0 wt % of lubricant by weight of the pharmaceutical formulation; preferably from 0.2 wt % to 2.7 wt %; preferably from 0.5 wt % to 2.5 wt % of lubricant by the total weight of the pharmaceutical formulation; and
   I from 1 wt % to 15 wt % of filler by weight of the pharmaceutical formulation; preferably from 1.3 wt % to 12 wt %; preferably from 2.5 wt % to 8 wt %; more preferably from 3 to 5 wt % of filler by the total weight of the pharmaceutical formulation.
47. The compound for use according to any one of statements 1 to 46, wherein oral dosage form is a tablet.
48. The compound for use according to any one of statements 1 to 47, wherein the oral dosage form is a solid dosage form, preferably a tablet which comprises at least 0.1 mg of compound of formula (I), preferably at least 0.5 mg, preferably at least 1 mg, preferably at least 2 mg, preferably at least 3 mg, preferably at least 4 mg, preferably at least 5 mg, preferably at least 6 mg, preferably at least 7 mg, preferably at least 8 mg, preferably at least 9 mg, preferably at least 10 mg, preferably at least 15 mg, preferably at least 20 mg, preferably at least 25 mg, preferably at least 30 mg, preferably at least 35 mg of compound of formula (I).
49. The compound for use according to any one of statements 1 to 48, wherein the oral dosage form is a solid dosage form, preferably a tablet, said tablet preferably comprising from 0.5 to 1500 mg of compound of formula (I); preferably from 1 to 1400 mg of compound of formula (I); preferably from 2 to 1300 mg of compound of formula (I); preferably from 3 to 1200 mg of compound of formula (I); preferably from 4 to 1250 mg of compound of formula (I); preferably from 5 to 1000 mg of compound of formula (I); preferably from 6 to 1000 mg of compound of formula (I); preferably from 7 to 1300 mg of compound of formula (I); preferably from 8 to 1200 mg of compound of formula (I); preferably from 9 to 1000 mg of compound of formula (I); preferably from 10 to 950 mg of compound of formula (I), preferably from 15 to 900 mg of compound of formula (I), preferably from 20 to 800 mg of compound of formula (I), preferably from 25 to 700 mg of compound of formula (I), preferably from 30 to 600 mg of compound of formula (I), preferably from 35 to 500 mg of compound of formula (I), preferably from 37 to 400 mg of compound of formula (I), preferably from 38 to 300 mg of compound of formula (I), preferably from 40 to 250 mg of compound of formula (I), preferably from 10 to 200 mg of compound of formula (I), preferably from 10 to 100 mg of compound of formula (I), preferably from 10 to 50 mg, or any particular amount or range comprised therein of compound of formula (I).
50. The compound for use according to any one of statements 1 to 49, wherein the oral administration of the oral dosage form results in a maximum concentration (Cₘₐₓ) of compound of formula (I) in plasma of a subject, preferably a human subject, of at least 10 μg/L, preferably at least 20 μg/L, preferably at least 30 μg/L, preferably at least 40 μg/L, preferably at least 50 μg/L, preferably at least 100 μg/L, preferably at least 150 μg/L, preferably from 25 μg/L to 10,000 μg/L; preferably from 50 μg/L to 5000 μg/L; preferably of from 60 μg/L to 4800 μg/L; preferably of from 70 μg/L to 4700 μg/L; preferably of from 80 μg/L to 4600 μg/L; preferably of from 90 μg/L to 4300 μg/L; preferably of from 100 μg/L to 4000 μg/L or any particular amount or range comprised therein.
51. The compound for use according to any one of statements 1 to 50, wherein the oral administration of the oral dosage form to a human in a fed state results in a maximum concentration (Cₘₐₓ) of compound of formula (I) in plasma of a subject, preferably a human subject of at least 50 μg/L, preferably at least 100 μg/L, preferably at least 150 μg/L, preferably from 25 μg/L to 10,000 μg/L; preferably from 50 μg/L to 5000 μg/L; preferably of from 70 μg/L to 4800 μg/L; preferably of from 80 μg/L to 4700 μg/L; preferably of from 90 μg/L to 4600 μg/L; preferably of from 100 μg/L to 4300 μg/L; preferably of from 130 μg/L to 4000 μg/L.
52. The compound for use according to any one of statements 1 to 51, wherein the oral dosage form is administered to the human subject at least once a day, for example at least twice a day, at least three times a day, for example at least four times a day.
53. The compound for use according to any one of statements 1 to 52, wherein the oral dosage form is administered to the human subject once or twice a day.
54. The compound for use according to any one of statements 1 to 53, wherein the amount of compound administered in one day or 24 hours (daily dose) is administered to the subject, preferably a human subject, in a single oral dosage form or is administered in two, three, or four oral dosage forms. For example, wherein the oral dosage form is a tablet, the daily dose may be administered to the subject in one tablet, or in two, three, or four tablets.
55. The compound for use according to any one of statements 1 to 54, wherein the compound of formula (I) is:

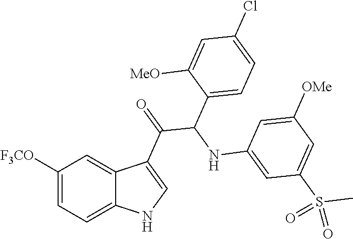 [SMILES: COc1cc(NC(C(=O)c2c[nH]c3ccc(OC(F)(F)F)cc23)c2ccc(Cl)cc2OC)cc(S(C)(=O)=O)c1]

or a stereo-isomeric form, a pharmaceutically acceptable salt, solvate or polymorph thereof.
56. The compound for use according to any one of statements 1 to 55, wherein the oral dosage form is administered following a dosage regimen comprising:
   1) at least one loading phase during which at least one dose (A) of compound of formula (I) is administered at least once a day over a time period of at least one day; and
   2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day over a time period of at least one day;
   wherein dose (A) is either higher or lower than dose (B). Preferably, dose (A) is higher than dose (B).
57. The compound for use according to any one of statements 1 to 56, wherein the oral dosage form is administered following a dosage regimen comprising:
   1) at least one loading phase during which at least one dose (A) of compound of formula (I) is administered at least once a day over a time period of at most 20 days, preferably at most 19 days, preferably at most 18 days, preferably at most 17 days, preferably at most 16 days, preferably at most 15 days, preferably at most 14, preferably at most 13 days, preferably at most 12 days, preferably at most 11 days, preferably at most 10 days, preferably at most 9 days, preferably at most 8 days, preferably at most 7 days, preferably at most 6 days, preferably at most 5 days, preferably at most 4 days, preferably at most 3 days, preferably at most 2 days, preferably at most 1 day, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, preferably at least twice every week, preferably at least once a day over a time period of at least one day to at most 12 months, preferably at most 9 months, preferably at most 6 months, preferably at most 3 months, preferably at most 1.5 months, preferably at most 40 days, preferably at most 39 days, preferably at most 38 days, preferably at most 37 days, preferably at most 36 days, preferably at most 35 days, preferably at most 30 days, preferably at most 28 days, preferably at most 21 days, preferably at most 14 days, preferably at most 7 days, preferably at most 5 days, or any particular amount or range comprised therein;
   wherein dose (A) is either higher or lower than dose (B). Preferably, dose (A) is higher than dose (B).
58. The compound for use according to any one of statements 1 to 57, wherein the oral dosage form is administered following a dosage regimen comprising:
   1) at least one loading phase during which at least one dose (A) of compound of formula (I) is administered at least once a day over a time period of at most 20, 19, 18, 17, 16, 15, 14, 13, 12, 11, 10, 9, 8, 7, 6, 5, 4, 3, 2, or 1 days, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day over a time period of at least one day to at most 12 months, 9 months, 6 months, 3 months, 1.5 months, 40 days, 39 days, 38 days, 37 days, 36 days, 35 days, 30 days, 28 days, 21 days, 14 days, 7 days, 5 days, or any particular amount or range comprised therein;
   wherein dose (A) is either higher or lower than dose (B). Preferably, dose (A) is higher than dose (B).
59. The compound for use according to any one of statements 1 to 58, wherein the oral dosage form is administered following a dosage regimen comprising:
   1) at least one loading phase during which at least one dose (A) of compound of formula (I) is administered at least once a day, preferably at least twice a day, preferably at least three times a day, or preferably at least four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, preferably at least once every week, preferably at least twice every week, preferably at least once a day, preferably at least twice a day, preferably at least three times a day, preferably at least four times a day over a time period of at least one day to at most 12 months, at least 2 days to at most 6 months, preferably at least 3 days to at most 3 months, preferably at least one day to at most 30 days, preferably over a time period of at least 5 days to at most 28 days, preferably over a time period of at least 10 days to at most 26 days or any particular amount or range comprised therein;
   wherein dose (A) is either higher or lower than dose (B). Preferably, dose (A) is higher than dose (B).
60. The compound for use according to any one of statements 56 to 59, wherein dose (A) of compound of formula (I) ranges from 50 mg to 1200 mg; preferably from 100 mg to 1100 mg; preferably from 150 mg to 900 mg; preferably from 50 mg to 800 mg; preferably from 60 mg to 700 mg; preferably from 70 mg to 650 mg; preferably from 80 mg to 600 mg; preferably from 90 mg to 550 mg; preferably from 100 mg to 500 mg or any particular amount or range comprised therein.
61. The compound for use according to any one of statements 56 to 60, wherein dose (A) of compound of formula (I) ranges from 5 mg to 1500 mg; preferably from 10 mg to 1400 mg; preferably from 20 mg to 1300 mg; preferably from 25 mg to 1200 mg; preferably from 30 mg to 1100 mg; preferably from 35 mg to 1000 mg; preferably from 15 mg to 1450 mg; preferably from 15 mg to 1100 mg; preferably from 25 mg to 1000 mg; preferably from 10 mg to 900 mg; preferably from 15 mg to 900 mg; preferably from 20 mg to 900 mg; preferably from 30 mg to 900 mg; preferably from 35 mg to 800 mg; preferably from 50 mg to 800 mg, or any particular amount or range comprised therein.
62. The compound for use according to any one of statements 56 to 61, wherein dose (A) of compound of formula (I) is administered at least once a day; preferably at least twice a day.
63. The compound for use according to any one of statements 56 to 62, wherein dose (B) of compound of formula (I) ranges from 5 mg to 1500 mg; preferably from 25 mg to 1200 mg; preferably from 50 mg to 1100 mg; preferably from 100 mg to 1000 mg; preferably from 150 mg to 900 mg; preferably from 5 mg to 500 mg; preferably from 5 mg to 450 mg; preferably from 5 mg to 400 mg; preferably from 5 mg to 350 mg; preferably from 10 mg to 300 mg; preferably from 15 mg to 250 mg; preferably from 20 mg to 230 mg; preferably from 30 mg to 250 mg; preferably from 35 mg to 200 mg; preferably from 50 mg to 450 mg, or any particular amount or range comprised therein.
64. The compound for use according to any one of statements 56 to 63, wherein dose (B) of compound of formula (I) is administered at least once every two weeks, preferably at least once every week, preferably at least twice every week, preferably at least once a day, preferably at least twice a day. Preferably dose (B) is administered once a day.
65. The compound for use according to any one of statements 56 to 64, wherein dose (B) of compound of formula (I) is administered at least once every two weeks, preferably at least once every week, preferably at least twice every week, preferably at least once a day, preferably at least twice a day, preferably at least three times a day, preferably at least four times a day over a time period of at least one day to at most 12 months, at least 2 days to at most 6 months, preferably at least 3 days to at most 3 months, preferably at least one day to at most 30 days, preferably over a time period of at least 5 days to at most 28 days, preferably over a time period of at least 10 days to at most 26 days or any particular amount or range comprised therein.
66. The compound for use according to any one of statements 56 to 65, wherein dose (A) of compound of formula (I) is administered at least once a day, preferably at least twice a day, preferably at least three times a day, or preferably at least four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein.
67. The compound for use according to any one of statements 56 to 66, wherein dose (A) of compound of formula (I) is administered at least once a day, preferably at least twice a day, preferably at least three times a day, or preferably at least four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein; 20 and wherein dose (A) of compound of formula (I) ranges from 50 mg to 1200 mg; preferably from 100 mg to 1100 mg; preferably from 150 mg to 900 mg; preferably from 50 mg to 800 mg; preferably from 60 mg to 700 mg; preferably from 70 mg to 650 mg; preferably from 80 mg to 600 mg; preferably from 90 mg to 550 mg; preferably from 100 mg to 500 mg or any particular amount or range comprised therein.
68. The compound for use according to any one of statements 56 to 67, wherein dose (A) of compound of formula (I) is administered at least once a day, preferably at least twice a day, preferably at least three times a day, or preferably at least four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein;
   and wherein dose (A) of compound of formula (I) ranges 5 mg to 1500 mg; preferably from 10 mg to 1400 mg; preferably from 20 mg to 1300 mg; preferably from 25 mg to 1200 mg; preferably from 30 mg to 1100 mg; preferably from 35 mg to 1000 mg; preferably from 15 mg to 1450 mg; preferably from 15 mg to 1100 mg; preferably from 25 mg to 1000 mg; preferably from 10 mg to 900 mg; preferably from 15 mg to 900 mg; preferably from 20 mg to 900 mg; preferably from 30 mg to 900 mg; preferably from 35 mg to 800 mg; preferably from 50 mg to 800 mg, or any particular amount or range comprised therein.
69. The compound for use according to any one of statements 56 to 68, wherein dose (B) of compound of formula (I) is administered at least once every two weeks, preferably at least once every week, preferably at least twice every week, preferably at least once a day, preferably at least twice a day, preferably at least three times a day, preferably at least four times a day over a time period of at least one day to at most 12 months, at least 2 days to at most 6 months, preferably at least 3 days to at most 3 months, preferably at least one day to at most 30 days, preferably over a time period of at least 5 days to at most 28 days, preferably over a time period of at least 10 days to at most 26 days or any particular amount or range comprised therein;
   and wherein dose (B) of compound of formula (I) ranges from 5 mg to 1500 mg; preferably from 25 mg to 1200 mg; preferably from 50 mg to 1100 mg; preferably from 100 mg to 1000 mg; preferably from 150 mg to 900 mg; preferably from 5 mg to 500 mg; preferably from 5 mg to 450 mg; preferably from 5 mg to 400 mg; preferably from 5 mg to 350 mg; preferably from 10 mg to 300 mg; preferably from 15 mg to 250 mg; preferably from 20 mg to 230 mg; preferably from 30 mg to 250 mg; preferably from 35 mg to 200 mg; preferably from 50 mg to 450 mg, or any particular amount or range comprised therein.
70. A pharmaceutical formulation for use in the prevention and/or treatment of dengue viral infection, said formulation comprising
   a) a compound of formula (I); and
   b1) methacrylic acid copolymer, or
   b2) a cellulose derivative such as methyl cellulose (MC), ethyl cellulose (EC), hydroxyethyl cellulose (HEC), hydroxypropyl cellulose (HPC), carboxymethyl cellulose (CMC), sodium carboxymethyl cellulose (NaCMC) or hydroxypropyl methylcellulose (HPMC), or a combination thereof. Preferably the cellulose derivative is HPMC;
   wherein the pharmaceutical formulation is administered to a human subject in either a fed or fasted state;
   wherein formula (I) corresponds to

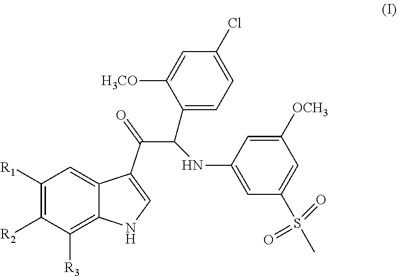

a stereo-isomeric form, a pharmaceutically acceptable salt, solvate or polymorph thereof;
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃.
71. The pharmaceutical formulation for use according to statement 70, wherein the pharmaceutical formulation is administered in an oral dosage form to a human subject in a fed state; preferably the human subject in a fed state has eaten a standard normal fat meal, a high fat-high calorie meal, a low fat-low calorie meal, or a low fat-high calorie meal.
72. The pharmaceutical formulation for use according to any one of statements 70 to 71, wherein said human subject is infected with Dengue virus or at risk of being infected with Dengue virus.
73. The pharmaceutical formulation for use according to any one of statements 70 or 72, wherein said human subject is in a fed state before administration of said oral dosage form or simultaneously with administration of said oral dosage form.
74. The pharmaceutical formulation for use according to any one of statements 70 to 73, wherein the pharmaceutical formulation is administered to a human subject in a fed state, and wherein said human in a fed state and has eaten less than 4 hours, preferably less than 3 hours, preferably less than 2 hours, preferably less than 1 hour, preferably less than 30 minutes, preferably less than 15 minutes, preferably less than 10 minutes before the time of administering the pharmaceutical formulation, preferably the pharmaceutical formulation is administered at most within 30 minutes of eating; preferably at most within 25 minutes of eating; preferably at most within 20 minutes of eating; preferably at most within 15 minutes of eating; preferably at most within 10 minutes of eating; preferably at most within 5 minutes of eating; preferably simultaneously with eating.
75. The pharmaceutical formulation for use according to any one of statements 70 to 74, wherein the pharmaceutical formulation is administered orally.
76. The pharmaceutical formulation for use according to any one of statements 70 to 75, wherein the pharmaceutical formulation is administered in the morning. Said oral dosage form may be administered simultaneously or after the first meal, the second meal or the third meal of the day.
77. The pharmaceutical formulation for use according to any one of statements 70 to 76, wherein said human in a fed state has eaten food comprising at least 0.1 g of fat, preferably at least 0.2 g, preferably at least 0.3 g, preferably at least 0.4 g, preferably at least 0.5 g, preferably at least 0.6 g, preferably at least 0.7 g, preferably at least 0.8 g, preferably at least 0.9 g of fat, preferably at least 1 g of fat, preferably at least 2 g, preferably at least 3 g, preferably at least 4 g, preferably at least 5 g of fat, preferably at least 6 g of fat, preferably at least 7 g, preferably at least 8 g, preferably at least 9 g, preferably at least 10 g of fat, preferably at least 11 g of fat, preferably at least 12 g, preferably at least 13 g, preferably at least 14 g, preferably at least 15 g of fat; preferably at least 16 g of fat, preferably at least 17 g, preferably at least 18 g, preferably at least 19 g, preferably at least 20 g of fat; preferably at least 21 g of fat, preferably at least 22 g, preferably at least 23 g, preferably at least 24 g, preferably at least 25 g of fat; preferably at least 26 g of fat, preferably at least 27 g, preferably at least 28 g, preferably at least 29 g, preferably at least 30 g of fat; preferably at least 31 g of fat, preferably at least 32 g, preferably at least 33 g, preferably at least 34 g, preferably at least 35 g of fat; preferably at least 36 g of fat, preferably at least 37 g, preferably at least 38 g, preferably at least 39 g, preferably at least 40 g of fat; preferably at least 41 g of fat, preferably at least 42 g, preferably at least 43 g, preferably at least 44 g, preferably at least 45 g of fat; preferably at least 46 g of fat, preferably at least 47 g, preferably at least 48 g, preferably at least 49 g, preferably at least 50 g of fat; preferably at least 51 g of fat, 52 g, preferably at least 53 g, preferably at least 54 g, preferably at least 55 g of fat; preferably at least 56 g of fat, preferably at least 57 g, preferably at least 58 g, preferably at least 59 g, preferably at least 60 g of fat, or any particular amount or range comprised therein.
78. The pharmaceutical formulation for use according to any one of statements 70 to 77, wherein said human in a fed state has eaten food comprising at least 0.1 g of fat, preferably at least 0.2 g, preferably at least 0.3 g, preferably at least 0.4 g, preferably at least 0.5 g, preferably at least 0.6 g, preferably at least 0.7 g, preferably at least 0.8 g, preferably at least 0.9 g of fat, preferably at least 1 g of fat, preferably at least 2 g, preferably at least 3 g, preferably at least 4 g, preferably at least 5 g of fat, preferably at least 6 g of fat, preferably at least 7 g, preferably at least 8 g, preferably at least 9 g, preferably at least 10 g of fat, preferably at least 11 g of fat, preferably at least 12 g, preferably at least 13 g, preferably at least 14 g, preferably at least 15 g of fat; preferably at least 16 g of fat, preferably at least 17 g, preferably at least 18 g, preferably at least 19 g, preferably at least 20 g of fat; preferably at least 21 g of fat, preferably at least 22 g, preferably at least 23 g, preferably at least 24 g, preferably at least 25 g of fat; preferably at least 26 g of fat, preferably at least 27 g, preferably at least 28 g, preferably at least 29 g, preferably at least 30 g of fat; preferably at least 31 g of fat, preferably at least 32 g, preferably at least 33 g, preferably at least 34 g, preferably at least 35 g of fat; preferably at least 36 g of fat, preferably at least 37 g, preferably at least 38 g, preferably at least 39 g, preferably at least 40 g of fat; preferably at least 41 g of fat, preferably at least 42 g, preferably at least 43 g, preferably at least 44 g, preferably at least 45 g of fat; preferably at least 46 g of fat, preferably at least 47 g, preferably at least 48 g, preferably at least 49 g, preferably at least 50 g of fat; preferably at least 51 g of fat, 52 g, preferably at least 53 g, preferably at least 54 g, preferably at least 55 g of fat; preferably at least 56 g of fat, preferably at least 57 g, preferably at least 58 g, preferably at least 59 g, preferably at least 60 g of fat, or any particular amount or range comprised therein;
   and wherein the human has eaten less than 4 hours from administration of the compound of formula I, preferably less than 3 hours, preferably less than 2 hours, preferably less than 1 hour, preferably less than 30 minutes, preferably less than 15 minutes, preferably less than 10 minutes before the time of administering the oral dosage form, preferably the oral dosage form is administered at most within 30 minutes of eating; preferably at most within 25 minutes of eating; preferably at most within 20 minutes of eating; preferably at most within 15 minutes of eating; preferably at most within 10 minutes of eating; preferably at most within 5 minutes of eating from administration of the compound of formula I, such as Compound (a); preferably the compound of formula I, such as Compound (a), preferably the compound of formula I is administered simultaneously with eating.
79. The pharmaceutical formulation for use according to any one of statements 70 to 78, wherein said human in a fed state has eaten food comprising at least 0.1 g, 0.2 g, 0.3 g, 0.4 g, 0.5 g, 0.6 g, 0.7 g, 0.8 g or 0.9 g of fat, preferably at least 1 g, 2 g, 3 g, 4 g or 5 g of fat, preferably at least 6 g, 7 g, 8 g, 9 g or 10 g of fat, preferably at least 11 g, 12 g, 13 g, 14 g or 15 g of fat; preferably at least 16 g, 17 g, 18 g, 18 g, 19 g or 20 g of fat; preferably at least 21 g, 22 g, 23 g, 24 g or 25 g of fat; preferably at least 26 g, 27 g, 28 g, 29 g or 30 g of fat; preferably at least 31 g, 32 g, 33 g, 34 g, or 35 g of fat; preferably at least 36 g, 37 g, 38 g, 39 g, or 40 g of fat; preferably at least 41 g, 42 g, 43 g, 44 g, or 45 g of fat; preferably at least 46 g, 47 g, 48 g, 49 g, or 50 g of fat; preferably at least 51 g, 52 g, 53 g, 54 g, or 55 g of fat; preferably at least 56 g, 57 g, 58 g, 59 g, or 60 g of fat, or any particular amount or range comprised therein.
80. The pharmaceutical formulation for use according to any one of statements 70 to 79, wherein said human in a fed state has eaten food having a total energy count of at least 10 kcal, preferably at least 20 kcal, preferably at least 30 kcal, preferably at least 40 kcal, preferably at least 50 kcal, preferably at least 60 kcal, preferably at least 70 kcal, preferably at least 80 kcal, preferably at least 90 kcal, preferably at least 100 kcal, preferably at least 110 kcal, preferably at least 120 kcal, preferably at least 130 kcal, preferably at least 140 kcal, preferably at least 150 kcal, preferably at least 160 kcal, preferably at least 170 kcal, preferably at least 180 kcal, preferably at least 190 kcal, preferably at least 200 kcal, preferably at least 210 kcal, preferably at least 220 kcal, preferably at least 230 kcal, preferably at least 240 kcal, preferably at least 250 kcal, preferably at least 260 kcal, preferably at least 270 kcal, preferably at least 280 kcal, preferably at least 290 kcal, preferably at least 300 kcal, preferably at least 310 kcal, preferably at least 320 kcal, preferably at least 330 kcal, preferably at least 340 kcal, preferably at least 350 kcal, preferably at least 360 kcal, preferably at least 370 kcal, preferably at least 380 kcal, preferably at least 390 kcal, preferably at least 400 kcal, preferably at least 410 kcal, preferably at least 420 kcal, preferably at least 430 kcal, preferably at least 440 kcal, preferably at least 450 kcal, preferably at least 460 kcal, preferably at least 470 kcal, preferably at least 480 kcal, preferably at least 490 kcal, preferably at least 500 kcal or any particular amount or range comprised therein. Preferably said human in a fed state has eaten food having a total energy count of at most 1500 kcal, preferably at most 1400 kcal, preferably at most 1300 kcal, preferably at most 1200 kcal, preferably at most 1110 kcal, preferably at most 1000 kcal, preferably at most 980 kcal, preferably at most 960 kcal, preferably at most 940 kcal, preferably at most 920 kcal, preferably at most 810 kcal, preferably at most 820 kcal, preferably at most 830 kcal, preferably at most 840 kcal preferably at most 850 kcal, preferably at most 860 kcal, preferably at most 870 kcal, preferably at most 880 kcal, preferably at most 890 kcal preferably at most 900 kcal, preferably at most 710 kcal, preferably at most 720 kcal, preferably at most 730 kcal, preferably at most 740 kcal, preferably at most 750 kcal, preferably at most 760 kcal, preferably at most 770 kcal, preferably at most 780 kcal, preferably at most 790 kcal preferably at most 800 kcal, preferably at most 610 kcal, preferably at most 620 kcal, preferably at most 630 kcal, preferably at most 640 kcal, preferably at most 650 kcal, preferably at most 660 kcal, preferably at most 670 kcal, preferably at most 680 kcal, preferably at most 690 kcal, preferably at most 700 kcal, preferably at most 510 kcal, preferably at most 520 kcal, preferably at most 530 kcal, preferably at most 540 kcal, preferably at most 550 kcal, preferably at most 560 kcal, preferably at most 570 kcal, preferably at most 580 kcal, preferably at most 590 kcal, preferably at most 600 kcal or any particular amount or range comprised therein.
81. The pharmaceutical formulation for use according to any one of statements 70 to 80, wherein said human in a fed state has eaten food having a total energy count of at least 10 kcal, 20 kcal, 30 kcal, 40 kcal or 50 kcal, preferably at least 60 kcal, 70 kcal, 80 kcal, 90 kcal or 100 kcal, preferably at least 110 kcal, 120 kcal, 130 kcal, 140 kcal or 150 kcal, preferably at least 160 kcal, 170 kcal, 180 kcal, 190 kcal or 200 kcal, preferably at least 210 kcal, 220 kcal, 230 kcal, 240 kcal, 250 kcal, 260 kcal, 270 kcal, 280 kcal, 290 kcal or 300 kcal, preferably at least 310 kcal, 320 kcal, 330 kcal, 340 kcal, 350 kcal, 360 kcal, 370 kcal, 380 kcal, 390 kcal or 400 kcal, preferably at least 410 kcal, 420 kcal, 430 kcal, 440 kcal, 450 kcal, 460 kcal, 470 kcal, 480 kcal, 490 kcal or 500 kcal or any particular amount or range comprised therein. Preferably said human in a fed state has eaten food having a total energy count of at most 1500 kcal, 1400 kcal, 1300 kcal, 1200 kcal or 1110 kcal, preferably at most 1000 kcal, 980 kcal, 960 kcal, 940 kcal or 920 kcal, preferably at most 810 kcal, 820 kcal, 830 kcal, 840 kcal or 850 kcal, preferably at most 860 kcal, 870 kcal, 880 kcal, 890 kcal or 900 kcal, preferably at most 710 kcal, 720 kcal, 730 kcal, 740 kcal, 750 kcal, 760 kcal, 770 kcal, 780 kcal, 790 kcal or 800 kcal, preferably at most 610 kcal, 620 kcal, 630 kcal, 640 kcal, 650 kcal, 660 kcal, 670 kcal, 680 kcal, 690 kcal or 700 kcal, preferably at most 510 kcal, 520 kcal, 530 kcal, 540 kcal, 550 kcal, 560 kcal, 570 kcal, 580 kcal, 590 kcal or 600 kcal or any particular amount or range comprised therein.
82. The pharmaceutical formulation for use according to any one of statements 70 to 81, wherein the pharmaceutical formulation comprises a cellulose derivative (such as HPMC); preferably wherein the compound of formula (I) and said hydroxypropyl methylcellulose are present in the oral dosage form in a ratio of from 4:1 w/w to 1:5 w/w compound of formula (I): cellulose derivative, preferably from 3.5:1 w/w to 1:4.5 w/w compound of formula (I): cellulose derivative; preferably from 3:1 w/w to 1:4 w/w compound of formula (I): cellulose derivative; preferably from 2.5:1 w/w to 1:3.5 w/w compound of formula (I): cellulose derivative; preferably from 2:1 w/w to 1:3 w/w compound of formula (I): cellulose derivative; preferably from 2.5:1 w/w to 1:2.5 w/w compound of formula (I): cellulose derivative, preferably from 2:1 w/w to 1:2 w/w compound of formula (I): cellulose derivative; preferably from 1.5:1 w/w to 1:1.5 w/w compound of formula (I): cellulose derivative or a ratio comprised within any two ratios mentioned herein, or a ratio range or sub-range within any two ratios mentioned herein.
83. The pharmaceutical formulation for use according to any one of statements 70 to 82, wherein the pharmaceutical formulation comprises a cellulose derivative, preferably wherein said hydroxypropyl methylcellulose has a viscosity ranging between 3 and 5000 mPa·s, in 2 wt % solution in H2O at 25° C.; preferably between 3 and 500 mPa·s, in 2 wt % solution in H2O at 25° C., preferably between 3 and 50 mPa·s in 2 wt % solution in H2O at 25° C. Preferably said cellulose derivative is hydroxypropyl methylcellulose selected from HPMC E5, HPMC E6, HPMC E15, HPMC E50, and mixtures thereof.
84. The pharmaceutical formulation for use according to any one of statements 70 to 83, wherein the pharmaceutical formulation comprises methacrylic acid copolymer; preferably wherein the compound of formula (I) and the methacrylic acid copolymer are present in the solid dosage form in a ratio of from 4:1 w/w to 1:9 w/w, preferably from 3.9:1 w/w to 1:8 w/w; preferably from 3.8:1 w/w to 1:7 w/w; from 3.7:1 w/w to 1:6 w/w; preferably from 3:6:1 w/w to 1:5 w/w, preferably from 3.5:1 w/w to 1:4.5 w/w; preferably from 3:1 w/w to 1:4 w/w; preferably from 2.5:1 w/w to 1:3.5 w/w; preferably from 2:1 w/w to 1:3 w/w; preferably from 2.5:1 w/w to 1:2.5 w/w, preferably from 2:1 w/w to 1:2 w/w; preferably from 1.5:1 w/w to 1:1.5 w/w or a ratio comprised within any two ratios mentioned herein, or a ratio range or sub-range within any two ratios mentioned herein.
85. The pharmaceutical formulation for use according to any one of statements 70 to 84, wherein the pharmaceutical formulation comprises a copolymer selected from the group comprising a copolymer of methacrylic acid and methyl methacrylate; a copolymer of methacrylic acid and ethyl acrylate and mixture thereof.
86. The pharmaceutical formulation for use according to any one of statements 70 to 85, wherein said pharmaceutical formulation comprises at most 50 wt %, preferably at most 40 wt %, preferably at most 30 wt %, preferably at most 25 wt %, preferably at most 20 wt % or any particular amount or range comprised therein, of compound of formula (I) relative to the total weight of the formulation.
87. The pharmaceutical formulation for use according to any one of statements 70 to 86, wherein said pharmaceutical formulation comprises from 0.1 wt % to 50 wt % of compound of formula (I) relative to the total weight of the formulation, preferably from 1 wt % to 40 wt %, more preferably from 2.5 wt % to 30 wt %, most preferably from 5 wt % to 25 wt %, or any particular amount or range comprised therein of compound of formula (I) relative to the total weight of the formulation.
88. The pharmaceutical formulation for use according to any one of statements 70 to 87, wherein said pharmaceutical formulation further comprises one or more pharmaceutically acceptable excipients, preferably selected from the group comprising disintegrants, binders, diluents, lubricants, stabilizers, wetting agents, glidants, osmotic agents, colorants, plasticizers, coatings, fillers, surfactants and mixtures thereof.
89. The pharmaceutical formulation for use according to any one of statements 70 to 88, wherein said pharmaceutical formulation further comprises one or more pharmaceutically acceptable excipients; preferably wherein the formulation comprises at most 95 wt %, at most 90 wt %, at most 80 wt % of the one or more pharmaceutically acceptable excipients relative to the total weight of the formulation, preferably at most 70 wt. %, preferably at most 60 wt % of the one or more pharmaceutically acceptable excipients relative to the total weight of the formulation.
90. The pharmaceutical formulation for use according to any one of statements 70 to 89, wherein said pharmaceutical formulation further comprises one or more diluents; preferably wherein the formulation comprises at most 95 wt %, preferably at most 80 wt %, preferably at most 75 wt % of the diluent relative to the total weight of the formulation; and/or wherein the formulation comprises at least 5 wt %, preferably at least 10 wt %, preferably at least 15 wt % of the diluent relative to the total weight of the formulation.
91. The pharmaceutical formulation for use according to any one of statements 70 to 90, wherein said pharmaceutical formulation further comprises one or more disintegrants; preferably wherein the formulation comprises at most 30 wt %, preferably at most 20 wt %, preferably at most 15 wt %, preferably at most 10 wt % of the disintegrant relative to the total weight of the formulation; and/or wherein the formulation comprises at least 1 wt %, preferably at least 2.5 wt %, preferably at least 5 wt %, preferably at least 8 wt % of the disintegrant relative to the total weight of the formulation.
92. The pharmaceutical formulation for use according to any one of statements 70 to 91, wherein said pharmaceutical formulation further comprises one or more binders; preferably wherein the formulation comprises at most 50 wt %, preferably at most 45 wt %, preferably at most 40 wt %, more preferably at most 35 wt % of the binder relative to the total weight of the formulation; and/or wherein the formulation comprises at least 5 wt %, preferably at least 10 wt %, preferably at least 15 wt %, more preferably at least 20 wt %, most preferably 25 wt % of the binder relative to the total weight of the formulation.
93. The pharmaceutical formulation for use according to any one of statements 70 to 92, wherein said pharmaceutical formulation further comprises one or more lubricants; preferably wherein the formulation comprises at most 5.5 wt %, preferably at most 3.5 wt %, preferably at most 2 wt % of the lubricant relative to the total weight of the formulation; and/or wherein the formulation comprises at least 0.5 wt %, preferably at least 1 wt %, preferably at least 1.5 wt % of the lubricant relative to the total weight of the formulation.
94. The pharmaceutical formulation for use according to any one of statements 70 to 93, wherein said pharmaceutical formulation further comprises one or more wetting agent; preferably wherein the formulation comprises at most 5.5 wt %, preferably at most 3.5 wt %, preferably at most 2 wt % of the wetting agent relative to the total weight of the formulation; and/or wherein the formulation comprises at least 0.5 wt %, preferably at least 1 wt %, preferably at least 1.5 wt % of the wetting agent relative to the total weight of the formulation.
95. The pharmaceutical formulation for use according to any one of statements 70 to 94, wherein said pharmaceutical formulation further comprises one or more glidant; preferably wherein the formulation comprises at most 10 wt %, preferably at most 8 wt %, preferably at most 5 wt % of the glidant relative to the total weight of the formulation; and/or wherein the formulation comprises at least 0.1 wt %, preferably at least 1 wt %, preferably at least 2 wt % of the glidant relative to the total weight of the formulation.
96. The pharmaceutical formulation for use according to any one of statements 70 to 95, wherein said pharmaceutical formulation comprises a plurality of granules forming an intragranular phase of the formulation and one or more pharmaceutically acceptable excipients forming an extragranular phase of the formulation.
97. The pharmaceutical formulation for use according to any one of statements 70 to 96, wherein said pharmaceutical formulation comprises at least 15 wt % of intragranular phase by weight of the pharmaceutical formulation; preferably at least 20 wt %; preferably at least 28 wt %; preferably at least 34 wt % of intragranular phase by the total weight of the pharmaceutical formulation.
98. The pharmaceutical formulation for use according to any one of statements 70 to 97, wherein said pharmaceutical formulation comprises at most 99 wt % of intragranular phase by weight of the pharmaceutical formulation; preferably at most 93 wt %; preferably at most 85 wt %; preferably at most 80 wt %; preferably at most 74 wt %; preferably at most 73 wt %; preferably at most 67 wt %; preferably at most 63 wt %; preferably at most 60 wt %; preferably at most 53 wt % of intragranular phase by the total weight of the pharmaceutical formulation.
99. The pharmaceutical formulation for use according to any one of statements 70 to 98, wherein said pharmaceutical formulation comprises an intragranular phase comprising from 1 wt % to 70 wt % of compound of formula (I) by weight of the pharmaceutical formulation; preferably from 2 wt % to 60 wt %; preferably from 3 wt % to 55 wt %; preferably from 4 wt % to 50 wt %; preferably from 5 wt % to 45 wt %; preferably from 5 wt % to 40 wt %; preferably from 10 wt % to 35 wt %; preferably from 15 wt % to 30 wt % of compound of formula (I) by the total weight of the pharmaceutical formulation.
100. The pharmaceutical formulation for use according to any one of statements 70 to 99, wherein said pharmaceutical formulation comprises an intragranular phase comprising from 5 wt % to 60 wt %; preferably from 8 wt % to 50 wt %; preferably from 15 wt % to 45 wt % of methacrylic acid copolymer, cellulose derivative or a mixture thereof by the total weight of the pharmaceutical formulation. Preferably, the cellulose derivative is hydroxypropyl methylcellulose.
101. The pharmaceutical formulation for use according to any one of statements 70 to 100, wherein said pharmaceutical formulation comprises an intragranular phase comprising from 5 wt % to 60 wt %; preferably from 10 wt % to 60 wt %; preferably from 10 wt % to 50 wt %; preferably from 15 wt % to 50 wt % of filler by the total weight of the pharmaceutical formulation.
102. The pharmaceutical formulation for use according to any one of statements 70 to 101, wherein said pharmaceutical formulation comprises an intragranular phase comprising from 1 wt % to 10 wt %; preferably from 1 wt % to 9 wt %, preferably from 1.7 wt % to 8 wt % of disintegrant by the total weight of the pharmaceutical formulation.
103. The pharmaceutical formulation for use according any one of statements 70 to 102, wherein said pharmaceutical formulation comprises an intragranular phase comprising from 0.1 wt % to 5 wt %; preferably from 0.2 wt % to 4.7 wt %; preferably from 0.5 to 4.5 wt % of glidant by the total weight of the pharmaceutical formulation.
104. The pharmaceutical formulation for use according to any one of statements 70 to 103, wherein said pharmaceutical formulation comprises an intragranular phase comprising 0.1 wt % to 5 wt %; preferably from 0.2 wt % to 4.7 wt %; preferably from 0.5 to 4.5 wt % of surfactant by the total weight of the pharmaceutical formulation.
105. The pharmaceutical formulation for use according to any one of statements 70 to 104, wherein said pharmaceutical formulation comprises an intragranular phase comprising from 0.1 wt % to 3 wt %; preferably from 0.2 wt % to 2.7 wt %; preferably from 0.5 to 2.5 wt % of lubricant by the total weight of the pharmaceutical formulation.
106. The pharmaceutical formulation for use according to any one of statements 70 to 105, wherein said pharmaceutical formulation comprises an intragranular phase comprising:
   (i) from 5 wt % to 45 wt %; preferably from 5 wt % to 40 wt %; preferably from 10 wt % to 35 wt %; preferably from 15 wt % to 30 wt % of compound of formula (I) by weight of the pharmaceutical formulation;
   (ii) from 5 wt % to 60 wt %; preferably from 8 wt % to 50 wt %; preferably from 15 wt % to 45 wt % of methacrylic acid copolymer or hydroxypropyl methylcellulose by weight of the pharmaceutical formulation; and
   optionally (iii) from 5 wt % to 60 wt %; preferably from 10 wt % to 60 wt %; preferably from 10 wt % to 50 wt %; preferably from 15 wt % to 50 wt % of filler by weight of the pharmaceutical formulation;
   optionally (iv) from 1 wt % to 10 wt %; preferably from 1 wt % to 9 wt. %, preferably from 1.7 wt % to 8 wt % of disintegrant by weight of the pharmaceutical formulation;
   optionally (v) from 0.1 wt % to 5 wt %; preferably from 0.2 wt % to 4.7 wt %; preferably from 0.5 to 4.5 wt % of glidant by weight of the pharmaceutical formulation;
   optionally (vi) from 0.1 wt % to 5 wt %; preferably from 0.2 wt % to 4.7 wt %; preferably from 0.5 to 4.5 wt % of surfactant by weight of the pharmaceutical formulation; and
   optionally (vii) from 0.1 wt % to 30 wt %; preferably from 0.2 wt % to 2.7 wt %; preferably from 0.5 wt % to 2.5 wt % of lubricant by weight of the pharmaceutical formulation.
107. The pharmaceutical formulation for use according to any one of statements 70 to 106, wherein said pharmaceutical formulation comprises at least 5 wt % of extragranular phase by weight of the pharmaceutical formulation; preferably at least 7 wt %; preferably at least 10 wt %; preferably at least 15 wt %; preferably at least 20 wt %; preferably at least 28 wt %; preferably at least 34 wt % of extragranular phase by the total weight of the pharmaceutical formulation.
108. The pharmaceutical formulation for use according to any one of statements 70 to 107, wherein said pharmaceutical formulation comprises at most 74 wt % of extragranular phase by weight of the pharmaceutical formulation; preferably at most 73 wt %; preferably at most 67 wt %; preferably at most 63 wt %; preferably at most 53 wt %, preferably at most 40 wt % of extragranular phase by the total weight of the pharmaceutical formulation.
109. The pharmaceutical formulation for use according to any one of statements 70 to 108, wherein said pharmaceutical formulation comprises an extragranular phase comprising from 0.1 wt % to 5 wt % of disintegrant by weight of the pharmaceutical formulation; preferably from 0.2 wt % to 4.7 wt %; preferably from 0.5 wt % to 3.5 wt % of disintegrant by the total weight of the pharmaceutical formulation.
110. The pharmaceutical formulation for use according to any one of statements 70 to 109, wherein said pharmaceutical formulation comprises an extragranular phase comprising from 0.1 wt % to 3 wt % of lubricant by weight of the pharmaceutical formulation; preferably from 0.2 wt % to 2.7 wt %; preferably from 0.5 wt % to 2.5 wt % of lubricant by the total weight of the pharmaceutical formulation.
111. The pharmaceutical formulation for use according to any one of statements 70 to 110, wherein said pharmaceutical formulation comprises an extragranular phase comprising from 0.1 wt % to 55 wt % of filler by weight of the pharmaceutical formulation; preferably from 0.2 wt % to 50 wt %; preferably from 0.3 wt % to 45 wt %; preferably from 0.4 wt % to 40 wt %; preferably from 0.5 wt % to 35 wt %; preferably from 0.6 wt % to 30 wt %; preferably from 0.7 to 25 wt %; preferably from 0.8 wt % to 20 wt %; preferably from 1 wt % to 15 wt %; preferably from 1.3 wt % to 12 wt %; preferably from 2 wt % to 10 wt %; preferably from 2.5 wt % to 9 wt %; preferably from 3 wt % to 8 wt %; preferably from 4 wt % to 7 wt %; preferably from 5 wt % to 6 wt % of filler by the total weight of the pharmaceutical formulation.
112. The pharmaceutical formulation for use according to any one of statements 70 to 111, wherein said pharmaceutical formulation comprises an extragranular phase comprising:
   (a) from 0.1 wt % to 5 wt % of disintegrant by weight of the pharmaceutical formulation; preferably from 0.2 wt % to 4.7 wt %; preferably from 0.5 wt % to 3.5 wt %; more preferably from 1 wt % to 3 wt % of disintegrant by the total weight of the pharmaceutical formulation;
   (b) from 0.1 wt % to 3 wt % of lubricant by weight of the pharmaceutical formulation; preferably from 0.2 wt % to 2.7 wt %; preferably from 0.5 wt % to 2.5 wt % of lubricant by the total weight of the pharmaceutical formulation; and
   (c) from 1 wt % to 15 wt % of filler by weight of the pharmaceutical formulation; preferably from 1.3 wt % to 12 wt %; preferably from 2.5 wt % to 8 wt %; more preferably from 3 to 5 wt % of filler by the total weight of the pharmaceutical formulation.
113. The pharmaceutical formulation for use according to any one of statements 70 to 112, wherein pharmaceutical formulation is a tablet.
114. The pharmaceutical formulation for use according to any one of statements 70 to 113, wherein the pharmaceutical formulation, preferably a tablet, comprises at least 0.1 mg of compound of formula (I), preferably at least 0.5 mg, preferably at least 1 mg, preferably at least 2 mg, preferably at least 3 mg, preferably at least 4 mg, preferably at least 5 mg, preferably at least 6 mg, preferably at least 7 mg, preferably at least 8 mg, preferably at least 9 mg, preferably at least, preferably at least 10 mg, preferably at least 15 mg, preferably at least 20 mg, preferably at least 25 mg, preferably at least 30 mg, preferably at least 35 mg of compound of formula (I).
115. The pharmaceutical formulation for use according to any one of statements 70 to 114, wherein the pharmaceutical formulation, preferably a tablet, comprises from 0.5 to 1500 mg of compound of formula (I); preferably from 1 to 1400 mg of compound of formula (I); preferably from 2 to 1300 mg of compound of formula (I); preferably from 3 to 1200 mg of compound of formula (I); preferably from 40 to 1250 mg of compound of formula (I); preferably from 5 to 1000 mg of compound of formula (I); preferably from 6 to 1000 mg of compound of formula (I); preferably from 7 to 1300 mg of compound of formula (I); preferably from 8 to 1200 mg of compound of formula (I); preferably from 9 to 1000 mg of compound of formula (I); preferably from 10 to 950 mg of compound of formula (I), preferably from 15 to 900 mg of compound of formula (I), preferably from 20 to 800 mg of compound of formula (I), preferably from 25 to 700 mg of compound of formula (I), preferably from 30 to 600 mg of compound of formula (I), preferably from 35 to 500 mg of compound of formula (I), preferably from 37 to 400 mg of compound of formula (I), preferably from 38 to 300 mg of compound of formula (I), preferably from 40 to 250 mg of compound of formula (I), preferably from 10 to 200 mg of compound of formula (I), preferably from 10 to 100 mg of compound of formula (I), preferably from 10 to 50 mg of compound of formula (I), or any particular amount or range comprised therein of compound of formula (I).
116. The pharmaceutical formulation for use according to any one of statements 70 to 115, wherein the oral administration of the pharmaceutical formulation results in a maximum concentration (Cₘₐₓ) of compound of formula (I) in plasma of a subject, preferably a human subject, of at least 10 μg/L, preferably at least 20 μg/L, preferably at least 30 μg/L, preferably at least 40 μg/L, preferably at least 50 μg/L, preferably at least 100 μg/L, preferably at least 150 μg/L, preferably from 50 μg/L to 5000 μg/L; preferably of from 60 μg/L to 4800 μg/L; preferably of from 70 μg/L to 4700 μg/L; preferably of from 80 μg/L to 4600 μg/L; preferably of from 90 μg/L to 4300 μg/L; preferably of from 100 μg/L to 4000 μg/L or any particular amount or range comprised therein.
117. The pharmaceutical formulation for use according to any one of statements 70 to 116, wherein the oral administration of the pharmaceutical formulation to a human in a fed state results in a n maximum concentration (Cₘₐₓ) of compound of formula (I) in plasma of a subject, preferably a human subject, of at least 50 μg/L, preferably at least 100 μg/L, preferably at least 150 μg/L, preferably from 50 μg/L to 5000 μg/L; preferably of from 70 μg/L to 4800 μg/L; preferably of from 80 μg/L to 4700 μg/L; preferably of from 90 μg/L to 4600 μg/L; preferably of from 100 μg/L to 4300 μg/L; preferably of from 130 μg/L to 4000 μg/L or any particular amount or range comprised therein.
118. The pharmaceutical formulation for use according to any one of statements 70 to 117, wherein the pharmaceutical formulation is administered orally to the human at least once a day, for example at least twice a day, at least three times a day, for example at least four times a day.
119. The pharmaceutical formulation for use according to any one of statements 70 to 118, wherein the pharmaceutical formulation is administered orally to the human once or twice a day.
120. The pharmaceutical formulation for use according to any one of statements 70 to 119, wherein the amount of compound comprised in the formulation and administered in one day or 24 hours (daily dose) is administered to the subject, preferably a human subject, in a single oral dosage form or is administered in two, three, or four oral dosage forms. For example, wherein the oral dosage form is a tablet, the daily dose may be administered to the subject in one tablet, or in two, three, or four tablets.
121. The pharmaceutical formulation for use according to any one of statements 70 to 120, wherein the compound of formula (I) is:

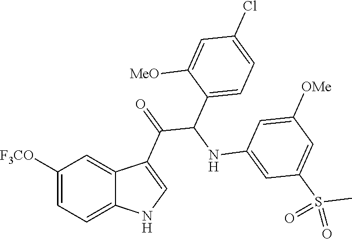 [SMILES: COc1cc(NC(C(=O)c2c[nH]c3ccc(OC(F)(F)F)cc23)c2ccc(Cl)cc2OC)cc(S(C)(=O)=O)c1]

or a stereo-isomeric form, a pharmaceutically acceptable salt, solvate or polymorph thereof.
122. The pharmaceutical formulation for use according to any one of statements 70 to 121, wherein the pharmaceutical formulation is administered following a dosage regimen comprising:
   1) at least one loading phase during which at least one dose (A) of compound of formula (I) is administered at least once a day over a time period of at least one day; and
   2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day over a time period of at least one day;
   wherein dose (A) is either higher or lower than dose (B). Preferably, dose (A) is higher than dose (B).
123. The pharmaceutical formulation for use according to any one of statements 70 to 122, wherein the pharmaceutical formulation is administered following a dosage regimen comprising:
   1) at least one loading phase during which at least one dose (A) of compound of formula (I) is administered at least once a day over a time period of most 20 days, preferably at most 19 days, preferably at most 18 days, preferably at most 17 days, preferably at most 16 days, preferably at most 15 days, preferably at most 14, preferably at most 13 days, preferably at most 12 days, preferably at most 11 days, preferably at most 10 days, preferably at most 9 days, preferably at most 8 days, preferably at most 7 days, preferably at most 6 days, preferably at most 5 days, preferably at most 4 days, preferably at most 3 days, preferably at most 2 days, preferably at most 1 day, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, preferably at least twice every week, preferably at least once a day over a time period of at least one day to at most 12 months, preferably to at most 9 months, preferably to at most 6 months, preferably to at most 3 months, preferably to at most 1.5 months, preferably to at most 40 days, preferably to at most 39 days, preferably to at most 38 days, preferably to at most 37 days, preferably to at most 36 days, preferably to at most 35 days, preferably to at most 30 days, preferably to at most 28 days, preferably to at most 21 days, preferably to at most 14 days, preferably to at most 7 days, preferably to at most 5 days, or any particular amount or range comprised therein;
   wherein dose (A) is either higher or lower than dose (B). Preferably, dose (A) is higher than dose (B).
124. The pharmaceutical formulation for use according to any one of statements 70 to 123, wherein the pharmaceutical formulation is administered following a dosage regimen comprising:
   1) at least one loading phase during which at least one dose (A) of compound of formula (I) is administered at least once a day over a time period of most 20, 19, 18, 17, 16, 15, 14, 13, 12, 11, 10, 9, 8, 7, 6, 5, 4, 3, 2, or 1 days, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day over a time period of at least one day to at most 12 months, 9 months, 6 months, 3 months, 1.5 months, 40 days, 39 days, 38 days, 37 days, 36 days, 35 days, 30 days, 28 days, 21 days, 14 days, 7 days, 5 days, or any particular amount or range comprised therein;
   wherein dose (A) is either higher or lower than dose (B). Preferably, dose (A) is higher than dose (B).
125. The pharmaceutical formulation for use according to any one of statements 70 to 124, wherein the pharmaceutical formulation is administered following a dosage regimen comprising:
   1) at least one loading phase during which at least one dose (A) of compound of formula (I) is administered at least once a day, preferably twice a day, preferably at least three times a day, preferably at least four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, preferably at least once every week, preferably at least twice every week, preferably at least once a day, preferably at least twice a day, preferably at least three times a day, preferably at least four times a day over a time period of at least one day to at most 12 months, preferably at least 2 days to at most 6 months, preferably at least 3 days to at most 3 months, preferably at least one day to at most 30 days, preferably over a time period of at least 5 days to at most 28 days, preferably over a time period of at least 10 days to at most 26 days or any particular amount or range comprised therein;
   wherein dose (A) is either higher or lower than dose (B). Preferably, dose (A) is higher than dose (B).
126. The pharmaceutical formulation for use according to any one of statements 70 to 125, wherein dose (A) of compound of formula (I) ranges from 50 mg to 1200 mg; preferably from 100 mg to 1100 mg; preferably from 150 mg to 900 mg; preferably from 50 mg to 800 mg; preferably from 60 mg to 700 mg; preferably from 70 mg to 650 mg; preferably from 80 mg to 600 mg; preferably from 90 mg to 550 mg; preferably from 100 mg to 500 mg or any particular amount or range comprised therein.
127. The pharmaceutical formulation for use according to any one of statements 70 to 126, wherein dose (A) of compound of formula (I) ranges from 5 mg to 1500 mg; preferably from 10 mg to 1400 mg; preferably from 20 mg to 1300 mg; preferably from 25 mg to 1200 mg; preferably from 30 mg to 1100 mg; preferably from 35 mg to 1000 mg; preferably from 15 mg to 1450 mg; preferably from 15 mg to 1100 mg; preferably from 25 mg to 1000 mg; preferably from 10 mg to 900 mg; preferably from 15 mg to 900 mg; preferably from 20 mg to 900 mg; preferably from 30 mg to 900 mg; preferably from 35 mg to 800 mg; preferably from 50 mg to 800 mg, or any particular amount or range comprised therein.
128. The pharmaceutical formulation for use according to any one of statements 70 to 127, wherein dose (A) of compound of formula (I) is administered at least once a day; preferably at least twice a day.
129. The pharmaceutical formulation for use according to any one of statements 70 to 128, wherein dose (B) of compound of formula (I) ranges from 5 mg to 1500 mg; preferably from 25 mg to 1200 mg; preferably from 50 mg to 1100 mg; preferably from 100 mg to 1000 mg; preferably from 150 mg to 900 mg; preferably from 5 mg to 500 mg; preferably from 5 mg to 450 mg; preferably from 5 mg to 400 mg; preferably from 5 mg to 350 mg; preferably from 10 mg to 300 mg; preferably from 15 mg to 250 mg; preferably from 20 mg to 230 mg; preferably from 30 mg to 250 mg; preferably from 35 mg to 200 mg, preferably from 50 mg to 450 mg, or any particular amount or range comprised therein.
130. The pharmaceutical formulation for use according to any one of statements 70 to 129, wherein dose (B) of compound of formula (I) is administered at least once every two weeks, preferably at least once every week, preferably at least twice every week, preferably at least once a day, preferably at least twice a day. Preferably dose (B) is administered once a day.
131. The pharmaceutical formulation for use according to any one of statements 70 to 130, wherein dose (B) of compound of formula (I) is administered at least once every two weeks, preferably at least once every week, preferably at least twice every week, preferably at least once a day, preferably at least twice a day, preferably at least three times a day, preferably at least four times a day over a time period of at least one day to at most 12 months, at least 2 days to at most 6 months, preferably at least 3 days to at most 3 months, preferably at least one day to at most 30 days, preferably over a time period of at least 5 days to at most 28 days, preferably over a time period of at least 10 days to at most 26 days or any particular amount or range comprised therein.
132. The pharmaceutical formulation for use according to any one of statements 70 to 131, wherein dose (A) of compound of formula (I) is administered at least once a day, preferably at least twice a day, preferably at least three times a day, or preferably at least four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein.
133. The pharmaceutical formulation for use according to any one of statements 70 to 132, wherein dose (A) of compound of formula (I) is administered at least once a day, preferably at least twice a day, preferably at least three times a day, or preferably at least four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein; and wherein dose (A) of compound of formula (I) ranges from 50 mg to 1200 mg; preferably from 100 mg to 1100 mg; preferably from 150 mg to 900 mg; preferably from 50 mg to 800 mg; preferably from 60 mg to 700 mg; preferably from 70 mg to 650 mg; preferably from 80 mg to 600 mg; preferably from 90 mg to 550 mg; preferably from 100 mg to 500 mg or any particular amount or range comprised therein.
134. The pharmaceutical formulation for use according to any one of statements 70 to 133, wherein dose (A) of compound of formula (I) is administered at least once a day, preferably at least twice a day, preferably at least three times a day, or preferably at least four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein;
   and wherein dose (A) of compound of formula (I) ranges 5 mg to 1500 mg; preferably from 10 mg to 1400 mg; preferably from 20 mg to 1300 mg; preferably from 25 mg to 1200 mg; preferably from 30 mg to 1100 mg; preferably from 35 mg to 1000 mg; preferably from 15 mg to 1450 mg; preferably from 15 mg to 1100 mg; preferably from 25 mg to 1000 mg; preferably from 10 mg to 900 mg; preferably from 15 mg to 900 mg; preferably from 20 mg to 900 mg; preferably from 30 mg to 900 mg; preferably from 35 mg to 800 mg; preferably from 50 mg to 800 mg, or any particular amount or range comprised therein.
135. The pharmaceutical formulation for use according to any one of statements 70 to 134, wherein dose (B) of compound of formula (I) is administered at least once every two weeks, preferably at least once every week, preferably at least twice every week, preferably at least once a day, preferably at least twice a day, preferably at least three times a day, preferably at least four times a day over a time period of at least one day to at most 12 months, at least 2 days to at most 6 months, preferably at least 3 days to at most 3 months, preferably at least one day to at most 30 days, preferably over a time period of at least 5 days to at most 28 days, preferably over a time period of at least 10 days to at most 26 days or any particular amount or range comprised therein;
   and wherein dose (B) of compound of formula (I) ranges from 5 mg to 1500 mg; preferably from 25 mg to 1200 mg; preferably from 50 mg to 1100 mg; preferably from 100 mg to 1000 mg; preferably from 150 mg to 900 mg; preferably from 5 mg to 500 mg; preferably from 5 mg to 450 mg; preferably from 5 mg to 400 mg; preferably from 5 mg to 350 mg; preferably from 10 mg to 300 mg; preferably from 15 mg to 250 mg; preferably from 20 mg to 230 mg; preferably from 30 mg to 250 mg; preferably from 35 mg to 200 mg; preferably from 50 mg to 450 mg, or any particular amount or range comprised therein.
136. A method of treating or preventing dengue viral infections, comprising administering to a human subject in need thereof (at risk of being infected by Dengue virus or infected by Dengue virus) a therapeutically effective amount of a compound of formula (I) for use according to any one of statements 1 to 69, or an effective amount of a pharmaceutical formulation for use according to any one of statements 70 to 135, wherein said compound of formula (I) or said pharmaceutical formulation is administered to the human subject in either a fed or fasted state and wherein formula (I) corresponds to

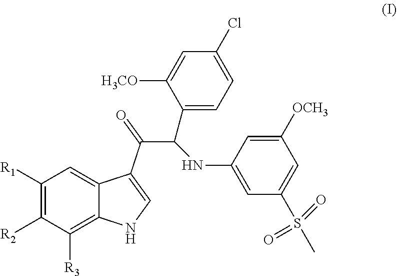

a stereo-isomeric form, a pharmaceutically acceptable salt, solvate or polymorph thereof;
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃.
137. The compound for use according to any one of statements 1 to 69, wherein the oral administration of the oral dosage form results in a maximum concentration (Cₘₐₓ) of compound of formula (I) in plasma of a subject, preferably a human subject, of at most 15,000 μg/L, preferably at most 12,000 μg/L, preferably at most 11,000 μg/L, preferably at most 10,000 μg/L, preferably at most 9500 μg/L, preferably at most 9000 μg/L, preferably at most 8500 μg/L, preferably from 25 μg/L to 15,000 μg/L; preferably from 50 μg/L to 12,000 μg/L; preferably of from 60 μg/L to 10,000 μg/L; preferably of from 70 μg/L to 9500 μg/L; preferably of from 80 μg/L to 8500 μg/L; preferably of from 90 μg/L to 8000 μg/L; preferably of from 100 μg/L to 8000 μg/L or any particular amount or range comprised therein.
138. The compound for use according to any one of statements 1 to 69 or 137, wherein the oral dosage form is administered following a dosage regimen comprising:
   1) at least one loading phase during which at least one dose (A) of compound of formula (I) is administered at least once; preferably twice a day; preferably three times a day; preferably four times a day over a time period of at most 20 days; preferably over a period of at most 19 days; preferably of at most 18 days; preferably of at most 17 days; preferably of at most 16 days; preferably of at most 15 days; preferably of at most 14 days; preferably of at most 13 days; preferably of at most 12 days; preferably of at most 11 days, preferably of at most 10 days, preferably of at most 9 days; preferably of at most 8 days; preferably of at most 7 days; preferably of at most 6 days; preferably of at most 5 days; preferably of at most 4 days; preferably of at most 3 days; preferably of at most 2 days; preferably of at most 1 day, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day, over a time period of at least one day; preferably over a time period of at least one day to at most 12 months, preferably over a time period of at most 9 months; preferably at most 6 months; preferably at most 3 months; preferably at most 1.5 months; preferably at most 40 days; preferably at most 39 days; preferably at most 38 days; preferably at most 37 days; preferably at most 36 days; preferably at most 35 days; preferably at most 30 days; preferably at most 28 days; preferably at most 21 days; preferably at most 14 days; preferably at most 7 days; preferably at most 5 days, or any particular amount or range comprised therein;
   wherein dose (A) is either higher or lower than dose (B); preferably, dose (A) is higher than dose (B).
139. The compound for use according to any one of statements 1 to 69 or 137 to 138, wherein dose (A) of compound of formula (I) ranges from 55 mg to 1200 mg; preferably from 50 mg to 1100 mg; preferably from 50 mg to 1000 mg; preferably from 55 mg to 1100 mg; preferably from 55 mg to 1000 mg; preferably from 55 mg to 900 mg; preferably from 60 mg to 1200 mg; preferably from 60 mg to 1100 mg; preferably from 60 m to 1000 mg; preferably from 60 mg to 800 mg; preferably from 60 mg to 750 mg; preferably from 70 mg to 700 mg; preferably from 75 mg to 600 mg; preferably from 85 mg to 550 mg; preferably from 50 mg to 500 mg or any particular amount or range comprised therein.
140. The compound for use according to any one of statements 1 to 69 or 137 to 139, wherein dose (A) of compound of formula (I) is administered at least once a day; preferably at least twice a day; preferably at least three times a day; preferably at least four times a day.
141. The compound for use according to any one of statements 1 to 69 or 137 to 140, wherein dose (A) of compound of formula (I) is administered at least once a day; preferably at least twice a day; preferably at least three times a day; preferably at least four times a day, over a time period of at least 1 day; preferably over a period of at least one day to at most 20 days; preferably over a period of at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein.
142. The compound for use according to any one of statements 1 to 69 or 137 to 141, wherein dose (B) of compound of formula (I) ranges from 5 mg to 1450 mg; preferably from 50 mg to 1500, preferably from 10 mg to 1300 mg, preferably from 10 mg to 1200 mg; preferably from 20 mg to 1100 mg; preferably from 25 mg to 1000 mg; preferably from 25 mg to 900 mg; preferably from 15 mg to 500 mg; preferably from 15 mg to 450 mg; preferably from 15 mg to 400 mg; preferably from 15 mg to 350 mg; preferably from 5 mg to 300 mg; preferably from 10 mg to 250 mg; preferably from 10 mg to 230 mg; preferably from 25 mg to 250 mg; preferably from 5 mg to 200 mg; preferably from 10 mg to 450 mg, or any particular amount or range comprised therein.
143. The compound for use according to any one of statements 1 to 69 or 137 to 142, wherein dose (B) of compound of formula (I) is administered at least once every two weeks, preferably at least once every week, preferably at least twice every week, preferably at least once a day, preferably at least twice a day; preferably dose (B) is administered once a day, over a time period of at least one day; preferably over a time period of at least one day to at most 12 months, preferably over a time period of at most 9 months; preferably at most 6 months; preferably at most 3 months; preferably at most 1.5 months; preferably at most 40 days; preferably at most 39 days; preferably at most 38 days; preferably at most 37 days; preferably at most 36 days; preferably at most 35 days; preferably at most 30 days; preferably at most 28 days; preferably at most 21 days; preferably at most 14 days; preferably at most 7 days; preferably at most 5 days, or any particular amount or range comprised therein.
144. The compound for use according to any one of statements 1 to 69 or 137 to 143, wherein the oral dosage form is administered following a dosage regimen comprising:
   1) at least one loading phase during which at least one dose (A) of compound of formula (I) is administered at least once a day over a time period of at least one day; and
   2) at least one maintenance phase starting on the next day following the last day of administration of dose (A) or the last day of the loading phase, during which at least one dose (B) of compound of formula (I), is administered at least once a day over a time period of at least one day;
   wherein dose (A) is either higher or lower than dose (B), preferably, dose (A) is higher than dose (B);
   wherein the oral dosage form, is a solid dosage form comprising a cellulose derivative having a viscosity ranging between 3 and 5000 mPa·s in 2 wt % solution in H2O at 25° C. and is selected from the group comprising HPMC E5, HPMC E6, HPMC E15, HPMC E50, HPMC K4M HPMC-AS and any combination thereof.
145. The compound for use according to any one of statements 1 to 69 or 137 to 144, wherein the oral dosage form is administered following a dosage regimen comprising:
   1) at least one loading phase during which at least one dose (A) of compound of formula (I) is administered at least once; preferably twice a day; preferably three times a day; preferably four times a day over a time period of at most 20 days; preferably over a period of at most 19 days; preferably of at most 18 days; preferably of at most 17 days; preferably of at most 16 days; preferably of at most 15 days; preferably of at most 14 days; preferably of at most 13 days; preferably of at most 12 days; preferably of at most 11 days, preferably of at most 10 days, preferably of at most 9 days; preferably of at most 8 days; preferably of at most 7 days; preferably of at most 6 days; preferably of at most 5 days; preferably of at most 4 days; preferably of at most 3 days; preferably of at most 2 days; preferably of at most 1 day, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day, over a time period of at least one day; preferably over a time period of at least one day to at most 12 months, preferably over a time period of at most 9 months; preferably at most 6 months; preferably at most 3 months; preferably at most 1.5 months; preferably at most 40 days; preferably at most 39 days; preferably at most 38 days; preferably at most 37 days; preferably at most 36 days; preferably at most 35 days; preferably at most 30 days; preferably at most 28 days; preferably at most 21 days; preferably at most 14 days; preferably at most 7 days; preferably at most 5 days, or any particular amount or range comprised therein;
      wherein dose (A) is either higher or lower than dose (B). Preferably, dose (A) is higher than dose (B);
      wherein the oral dosage form, is a solid dosage form comprising a cellulose derivative having a viscosity ranging between 3 and 5000 mPa·s in 2 wt % solution in H2O at 25° C. and is selected from the group comprising HPMC E5, HPMC E6, HPMC E15, HPMC E50, HPMC K4M HPMC-AS and any combination thereof.
146. The compound for use according to any one of statements 1 to 69 or 137 to 145, wherein the oral dosage form is administered following a dosage regimen comprising:
   1) at least one loading phase during which at least one dose (A) of compound of formula (I) is administered at least once a day, twice a day, three times a day or four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, at least twice every week, at least once a day, twice a day, three times a day or four times a day over a time period of at least one day to at most 12 months, at least 2 days to at most 6 months, at least 3 days to at most 3 months, at least one day to at most 30 days, preferably over a time period of at least 5 days to at most 28 days, preferably over a time period of at least 10 days to at most 26 days or any particular amount or range comprised therein;
      wherein dose (A) is either higher or lower than dose (B). Preferably, dose (A) is higher than dose (B);
      wherein the oral dosage form, is a solid dosage form comprising a cellulose derivative having a viscosity ranging between 3 and 5000 mPa·s in 2 wt % solution in H2O at 25° C. and is selected from the group comprising HPMC E5, HPMC E6, HPMC E15, HPMC E50, HPMC K4M HPMC-AS and any combination thereof.
147. The pharmaceutical formulation for use according to any one of statements 70 to 135, wherein the oral administration of the oral dosage form results in a maximum concentration (Cₘₐₓ) of compound of formula (I) in plasma of a subject, preferably a human subject, of at most 15,000 μg/L, preferably at most 12,000 μg/L, preferably at most 11,000 μg/L, preferably at most 10,000 μg/L, preferably at most 9500 μg/L, preferably at most 9000 μg/L, preferably at most 8500 μg/L, preferably from 25 μg/L to 15,000 μg/L; preferably from 50 μg/L to 12,000 μg/L; preferably of from 60 μg/L to 10,000 μg/L; preferably of from 70 μg/L to 9500 μg/L; preferably of from 80 μg/L to 8500 μg/L; preferably of from 90 μg/L to 8000 μg/L; preferably of from 100 μg/L to 8000 μg/L or any particular amount or range comprised therein.
148. The pharmaceutical formulation for use according to any one of statements 70 to 135, or 147, wherein the oral dosage form is administered following a dosage regimen comprising:
   1) at least one loading phase during which at least one dose (A) of compound of formula (I) is administered at least once; preferably twice a day; preferably three times a day; preferably four times a day over a time period of at most 20 days; preferably over a period of at most 19 days; preferably of at most 18 days; preferably of at most 17 days; preferably of at most 16 days; preferably of at most 15 days; preferably of at most 14 days; preferably of at most 13 days; preferably of at most 12 days; preferably of at most 11 days, preferably of at most 10 days, preferably of at most 9 days; preferably of at most 8 days; preferably of at most 7 days; preferably of at most 6 days; preferably of at most 5 days; preferably of at most 4 days; preferably of at most 3 days; preferably of at most 2 days; preferably of at most 1 day, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day, over a time period of at least one day; preferably over a time period of at least one day to at most 12 months, preferably over a time period of at most 9 months; preferably at most 6 months; preferably at most 3 months; preferably at most 1.5 months; preferably at most 40 days; preferably at most 39 days; preferably at most 38 days; preferably at most 37 days; preferably at most 36 days; preferably at most 35 days; preferably at most 30 days; preferably at most 28 days; preferably at most 21 days; preferably at most 14 days; preferably at most 7 days; preferably at most 5 days, or any particular amount or range comprised therein;
      wherein dose (A) is either higher or lower than dose (B). Preferably, dose (A) is higher than dose (B).
149. The pharmaceutical formulation for use according to any one of statements 70 to 135 or 147 to 148, wherein dose (A) of compound of formula (I) ranges from 55 mg to 1200 mg; preferably from 50 mg to 1100 mg; preferably from 50 mg to 1000 mg; preferably from 55 mg to 1100 mg; preferably from 55 mg to 1000 mg; preferably from 55 mg to 900 mg; preferably from 60 mg to 1200 mg; preferably from 60 mg to 1100 mg; preferably from 60 m to 1000 mg; preferably from 60 mg to 800 mg; preferably from 60 mg to 750 mg; preferably from 70 mg to 700 mg; preferably from 75 mg to 600 mg; preferably from 85 mg to 550 mg; preferably from 50 mg to 500 mg or any particular amount or range comprised therein.
150. The pharmaceutical formulation for use according to any one of statements 70 to 135 or 147 to 150, wherein dose (A) of compound of formula (I) is administered at least once a day; preferably at least twice a day; preferably at least three times a day; preferably at least four times a day.
151. The pharmaceutical formulation for use according to any one of statements 70 to 135 or 147 to 150, wherein dose (A) of compound of formula (I) is administered at least once a day; preferably at least twice a day; preferably at least three times a day; preferably at least four times a day, over a time period of at least 1 day; preferably over a period of at least one day to at most 20 days; preferably over a period of at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein.
152. The pharmaceutical formulation for use according to any one of statements 70 to 135 or 147 to 151, wherein dose (B) of compound of formula (I) ranges from 5 mg to 1450 mg; preferably from 50 mg to 1500, preferably from 10 mg to 1300 mg, preferably from 10 mg to 1200 mg; preferably from 20 mg to 1100 mg; preferably from 25 mg to 1000 mg; preferably from 25 mg to 900 mg; preferably from 15 mg to 500 mg; preferably from 15 mg to 450 mg; preferably from 15 mg to 400 mg; preferably from 15 mg to 350 mg; preferably from 5 mg to 300 mg; preferably from 10 mg to 250 mg; preferably from 10 mg to 230 mg; preferably from 25 mg to 250 mg; preferably from 5 mg to 200 mg; preferably from 10 mg to 450 mg, or any particular amount or range comprised therein.
153. The pharmaceutical formulation for use according to any one of statements 70 to 135 or 147 to 152, wherein dose (B) of compound of formula (I) is administered at least once every two weeks, preferably at least once every week, preferably at least twice every week, preferably at least once a day, preferably at least twice a day; preferably dose (B) is administered once a day, over a time period of at least one day; preferably over a time period of at least one day to at most 12 months, preferably over a time period of at most 9 months; preferably at most 6 months; preferably at most 3 months; preferably at most 1.5 months; preferably at most 40 days; preferably at most 39 days; preferably at most 38 days; preferably at most 37 days; preferably at most 36 days; preferably at most 35 days; preferably at most 30 days; preferably at most 28 days; preferably at most 21 days; preferably at most 14 days; preferably at most 7 days; preferably at most 5 days, or any particular amount or range comprised therein.
154. The pharmaceutical formulation for use according to any one of statements 70 to 135 or 147 to 153, wherein the oral dosage form is administered following a dosage regimen comprising:
   1) at least one loading phase during which at least one dose (A) of compound of formula (I) is administered at least once a day over a time period of at least one day; and
   2) at least one maintenance phase starting on the next day following the last day of administration of dose (A) or the last day of the loading phase, during which at least one dose (B) of compound of formula (I), is administered at least once a day over a time period of at least one day;
   wherein dose (A) is either higher or lower than dose (B), preferably, dose (A) is higher than dose (B);
   wherein the oral dosage form, is a solid dosage form comprising a cellulose derivative having a viscosity ranging between 3 and 5000 mPa·s in 2 wt % solution in H₂O at 25° C. and is selected from the group comprising HPMC E5, HPMC E6, HPMC E15, HPMC E50, HPMC K4M HPMC-AS and any combination thereof.
155. The pharmaceutical formulation for use according to any one of statements 70 to 135 or 147 to 154, wherein the oral dosage form is administered following a dosage regimen comprising:
   1) at least one loading phase during which at least one dose (A) of compound of formula (I) is administered at least once; preferably twice a day; preferably three times a day; preferably four times a day over a time period of at most 20 days; preferably over a period of at most 19 days; preferably of at most 18 days; preferably of at most 17 days; preferably of at most 16 days; preferably of at most 15 days; preferably of at most 14 days; preferably of at most 13 days; preferably of at most 12 days; preferably of at most 11 days, preferably of at most 10 days, preferably of at most 9 days; preferably of at most 8 days; preferably of at most 7 days; preferably of at most 6 days; preferably of at most 5 days; preferably of at most 4 days; preferably of at most 3 days; preferably of at most 2 days; preferably of at most 1 day, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day, over a time period of at least one day; preferably over a time period of at least one day to at most 12 months, preferably over a time period of at most 9 months; preferably at most 6 months; preferably at most 3 months; preferably at most 1.5 months; preferably at most 40 days; preferably at most 39 days; preferably at most 38 days; preferably at most 37 days; preferably at most 36 days; preferably at most 35 days; preferably at most 30 days; preferably at most 28 days; preferably at most 21 days; preferably at most 14 days; preferably at most 7 days; preferably at most 5 days, or any particular amount or range comprised therein;
      wherein dose (A) is either higher or lower than dose (B). Preferably, dose (A) is higher than dose (B);
      wherein the oral dosage form, is a solid dosage form comprising a cellulose derivative having a viscosity ranging between 3 and 5000 mPa·s in 2 wt % solution in H₂O at 25° C. and is selected from the group comprising HPMC E5, HPMC E6, HPMC E15, HPMC E50, HPMC K4M HPMC-AS and any combination thereof.
156. The pharmaceutical formulation for use according to any one of statements 70 to 135 or 147 to 155, wherein the oral dosage form is administered following a dosage regimen comprising:
   1) at least one loading phase during which at least one dose (A) of compound of formula (I) is administered at least once a day, twice a day, three times a day or four times a day, over a time period of at least 1 day to at most 20 days; preferably at least 2 days to at most 19 days, preferably at least 3 days to at most 18 days, preferably at least 4 days to at most 17 days, preferably at least 5 days to at most 16 days, preferably at least 6 days to at most 15 days, preferably at least 7 days to at most 14 days, preferably at least 8 days to at most 13 days, preferably at least 9 days to at most 12 days, preferably at least 10 days to at most 11 days, or any particular amount or range comprised therein; and
   2) at least one maintenance phase starting on the next day following the last day of administration of dose (A) or the last day of the loading phase, during which at least one dose (B) of compound of formula (I), is administered at least once every two weeks, at least once every week, at least twice every week, at least once a day, twice a day, three times a day or four times a day over a time period of at least one day to at most 12 months, at least 2 days to at most 6 months, at least 3 days to at most 3 months, at least one day to at most 30 days, preferably over a time period of at least 5 days to at most 28 days, preferably over a time period of at least 10 days to at most 26 days or any particular amount or range comprised therein;
      wherein dose (A) is either higher or lower than dose (B). Preferably, dose (A) is higher than dose (B);
      wherein the oral dosage form, is a solid dosage form comprising a cellulose derivative having a viscosity ranging between 3 and 5000 mPa·s in 2 wt % solution in H₂O at 25° C. and is selected from the group comprising HPMC E5, HPMC E6, HPMC E15, HPMC E50, HPMC K4M HPMC-AS and any combination thereof.

The present invention provides a compound of formula (I) for use in the prevention and/or treatment of dengue viral infections, wherein the compound is administered in an oral dosage form to a human in either a fed or fasted state and wherein formula (I) corresponds to

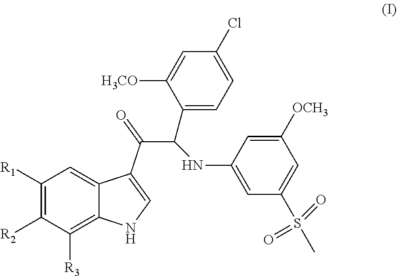

a stereo-isomeric form, a pharmaceutically acceptable salt, solvate or polymorph thereof; said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃.

In some embodiments, the oral dosage form is a solid dosage form formulated in a in a pharmaceutical formulation comprising a cellulose derivative such as hydroxypropyl methylcellulose (HPMC), or methacrylic acid copolymer or a combination thereof.

The present invention also provides a compound of formula (I) for use in the prevention and/or treatment of dengue viral infections, wherein the compound is administered in an oral dosage form to a human in either a fed or fasted state and wherein formula (I) corresponds to

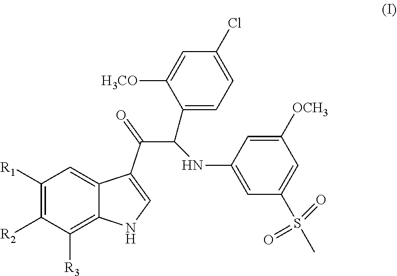

a stereo-isomeric form, a pharmaceutically acceptable salt, solvate or polymorph thereof;
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃;
wherein the oral dosage form is administered following a dosage regimen comprising:
1) at least one loading phase during which at least one dose (A) of compound of formula (I) is administered at least once; preferably twice a day; preferably three times a day; preferably four times a day over a time period of at most 20 days; preferably over a period of at most 19 days; preferably of at most 18 days; preferably of at most 17 days; preferably of at most 16 days; preferably of at most 15 days; preferably of at most 14 days; preferably of at most 13 days; preferably of at most 12 days; preferably of at most 11 days, preferably of at most 10 days, preferably of at most 9 days; preferably of at most 8 days; preferably of at most 7 days; preferably of at most 6 days; preferably of at most 5 days; preferably of at most 4 days; preferably of at most 3 days; preferably of at most 2 days; preferably of at most 1 day, or any particular amount or range comprised therein; and
2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day, over a time period of at least one day; preferably over a time period of at least one day to at most 12 months, preferably over a time period of at most 9 months; preferably at most 6 months; preferably at most 3 months; preferably at most 1.5 months; preferably at most 40 days; preferably at most 39 days; preferably at most 38 days; preferably at most 37 days; preferably at most 36 days; preferably at most 35 days; preferably at most 30 days; preferably at most 28 days; preferably at most 21 days; preferably at most 14 days; preferably at most 7 days; preferably at most 5 days, or any particular amount or range comprised therein;
wherein dose (A) is either higher or lower than dose (B); preferably, dose (A) is higher than dose (B).

In some embodiments, the human subject in a fed state has eaten a standard normal fat meal, a high fat-high calorie meal, a low fat-low calorie meal, or a low fat-high calorie meal.

In some embodiments the compound of formula (I) is administered to an individual at risk of being infected by Dengue virus. Said individual may be living in or traveling to a dengue endemic region. The compound of formula (I) may be also administered to an individual who is already infected by Dengue virus. In some embodiments, the human subject is infected with Dengue virus or at risk of being infected with Dengue virus.

In some embodiments at most 30% of subjects, particularly human subjects, in a fasting or fed state experience treatment emergent adverse events related to the administration a compound of formula (I), in particular Compound (a); preferably at most 25%; preferably at most 20%; preferably at most 19% of subjects; preferably at most 18% of subjects; preferably at most 17% of subjects; preferably at most 15% of subjects; preferably at most 14% of subjects; preferably at most 13% of subjects in a fasting or fed state experience treatment emergent adverse events related to the administration a compound of formula (I), in particular of Compound (a).

In some embodiments at most 30% of subjects, particularly human subjects, in a fasting or fed state experience treatment emergent adverse events related to the administration a compound of formula (I), in particular Compound (a), wherein said treatment emergent adverse events are of Grade 2 or lower; preferably at most 25%; preferably at most 20%; preferably at most 19% of subjects; preferably at most 18% of subjects; preferably at most 17% of subjects; preferably at most 15% of subjects; preferably at most 14% of subjects in a fasting or fed state experience treatment emergent adverse events related to the administration a compound of formula (I), in particular Compound (a), wherein said treatment emergent adverse events are of Grade 2 or lower.

In some embodiments at most 30% of subjects, particularly human subjects, in a fasting or fed state experience treatment emergent adverse events related to the administration a compound of formula (I), in particular Compound (a); preferably at most 25%; preferably at most 20%; preferably at most 19% of subjects; preferably at most 18% of subjects; preferably at most 17% of subjects; preferably at most 15% of subjects; preferably at most 14% of subjects; preferably at most 13% of subjects in a fed state experience treatment emergent adverse events related to the administration a compound of formula (I), in particular of Compound (a).

In some embodiments at most 30% of subjects, particularly human subjects, in a fasting or fed state experience treatment emergent adverse events related to the administration a compound of formula (I), in particular Compound (a), wherein said treatment emergent adverse events are of Grade 2 or lower; preferably at most 25%; preferably at most 20%; preferably at most 19% of subjects; preferably at most 18% of subjects; preferably at most 17% of subjects; preferably at most 15% of subjects; preferably at most 14% of subjects in a fed state experience treatment emergent adverse events related to the administration a compound of formula (I), in particular Compound (a), wherein said treatment emergent adverse events are of Grade 2 or lower. In some embodiments at most 30% of subjects, particularly human subjects, in a fasting or fed state experience treatment emergent adverse events related to the administration a compound of formula (I), in particular Compound (a); preferably at most 25%; preferably at most 20%; preferably at most 19% of subjects; preferably at most 18% of subjects; preferably at most 17% of subjects; preferably at most 15% of subjects; preferably at most 14% of subjects; preferably at most 13% of subjects in a fasting state experience treatment emergent adverse events related to the administration a compound of formula (I), in particular of Compound (a).

In some embodiments at most 30% of subjects, particularly human subjects, in a fasting or fed state experience treatment emergent adverse events related to the administration a compound of formula (I), in particular Compound (a), wherein said treatment emergent adverse events are of Grade 2 or lower; preferably at most 25%; preferably at most 20%; preferably at most 19% of subjects; preferably at most 18% of subjects; preferably at most 17% of subjects; preferably at most 15% of subjects; preferably at most 14% of subjects in a fasting state experience treatment emergent adverse events related to the administration a compound of formula (I), in particular Compound (a), wherein said treatment emergent adverse events are of Grade 2 or lower.

The present invention also provides a compound of formula (I) for use in the prevention and/or treatment of dengue viral infections, wherein the compound is administered in an oral dosage form to a human in either a fed or fasted state and wherein formula (I) corresponds to

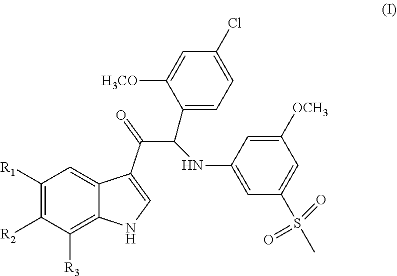

a stereo-isomeric form, a pharmaceutically acceptable salt, solvate or polymorph thereof;
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃;
   wherein the oral dosage form is administered following a dosage regimen comprising:
1) at least one loading phase during which at least one dose (A) of compound of formula (I) is administered at least once; preferably twice a day; preferably three times a day; preferably four times a day over a time period of at most 20 days; preferably over a period of at most 19 days; preferably of at most 18 days; preferably of at most 17 days; preferably of at most 16 days; preferably of at most 15 days; preferably of at most 14 days; preferably of at most 13 days; preferably of at most 12 days; preferably of at most 11 days, preferably of at most 10 days, preferably of at most 9 days; preferably of at most 8 days; preferably of at most 7 days; preferably of at most 6 days; preferably of at most 5 days; preferably of at most 4 days; preferably of at most 3 days; preferably of at most 2 days; preferably of at most 1 day, or any particular amount or range comprised therein; and
2) at least one maintenance phase starting on the next day following the last day of administration of dose (A) or the last day of the loading phase, during which at least one dose (B) of compound of formula (I), is administered at least once every two weeks, at least once every week, at least twice every week, or at least once a day, over a time period of at least one day; preferably over a time period of at least one day to at most 12 months, preferably over a time period of at most 9 months; preferably at most 6 months; preferably at most 3 months; preferably at most 1.5 months; preferably at most 40 days; preferably at most 39 days; preferably at most 38 days; preferably at most 37 days; preferably at most 36 days; preferably at most 35 days; preferably at most 30 days; preferably at most 28 days; preferably at most 21 days; preferably at most 14 days; preferably at most 7 days; preferably at most 5 days, or any particular amount or range comprised therein;
wherein dose (A) is either higher or lower than dose (B); preferably, dose (A) is higher than dose (B);
wherein the oral dosage form, is a solid dosage form comprising a cellulose derivative having a viscosity ranging between 3 and 5000 mPa·s in 2 wt % solution in H₂O at 25° C. and is selected from the group comprising HPMC E5, HPMC E6, HPMC E15, HPMC E50, HPMC K4M HPMC-AS and any combination thereof.

The present invention also provides a compound of formula (I) for use in the prevention and/or treatment of dengue viral infections, wherein the compound is administered in an oral dosage form to a human in either a fed or fasted state and wherein formula (I) corresponds to

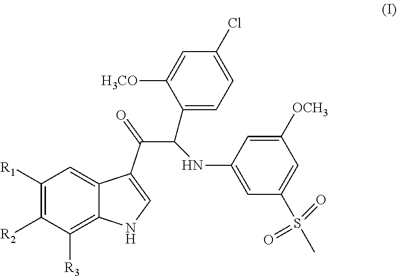

a stereo-isomeric form, a pharmaceutically acceptable salt, solvate or polymorph thereof;
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃;
wherein the oral dosage form, is a solid dosage form comprising a cellulose derivative having a viscosity ranging between 3 and 5000 mPa·s in 2 wt % solution in H₂O at 25° C. and is selected from the group comprising HPMC E5, HPMC E6, HPMC E15, HPMC E50, HPMC K4M HPMC-AS and any combination thereof.

The present invention also comprises a pharmaceutical formulation for use in the prevention and/or treatment of dengue viral infection, said formulation, comprising
11) a) a compound formula (I) as described herein; and
b1) methacrylic acid copolymer, or
b2) a cellulose derivative such as hydroxypropyl methylcellulose (HPMC);
wherein the pharmaceutical formulation is administered in an oral dosage form to a human in either a fed or fasted state. Said human is at risk of being infected by Dengue virus or is already infected by Dengue virus.

In some embodiments, the oral dosage form and/or pharmaceutical formulation according to the invention comprises a compound of formula (I) as described herein and hydroxypropyl methylcellulose.

In some embodiments, the pharmaceutical formulation according to the invention and/or the oral dosage form comprising the compound of formula (I) according to the invention is used for the prevention and/or treatment of dengue viral infections. In some embodiments, the pharmaceutical formulation and/or the oral dosage form is administered following a dosage regimen comprising: 1) at least one loading phase during which at least one dose (A) of compound of formula (I) is administered at least once a day over a time period of at least one day; and 2) at least one maintenance phase starting on the next day following the last day of administration of dose (A) or the last day of the loading phase, during which at least one dose (B) of compound of formula (I), is administered at least once every two weeks, preferably at least once every week, preferably at least twice every week, preferably at least once a day over a time period of at least one day; wherein dose (A) is either higher or lower than dose (B). Preferably, dose (A) is higher than dose (B).

In some embodiments, any of dose (A) or dose (B) may change throughout the days of the loading phase or the maintenance phase such as it increases and/or decreases during the days of said loading phase or the maintenance phase. For instance, any of dose (A) or dose (B) may be unchanged for the first 1 day, preferably the first 2 days, preferably the first 3 days, preferably the first 4 days, preferably the first 5 days, preferably the first 6 days, preferably the first 7 days, preferably the first 8 days, preferably the first 9 days, preferably the first 10 days, preferably the first 11 days, preferably the first 12 days, preferably the first 13 days, preferably the first 14 days, preferably the first 15 days, preferably the first 16 days, preferably the first 17 days, preferably the first 18 days, preferably the first 19 days, preferably the first 20 days, preferably the first 21 days, preferably the first 22 days, preferably the first 23 days, preferably the first 24 days, preferably the first 25 days, preferably the first 26 days, preferably the first 27 days, preferably the first 28 days, preferably the first 29 days, preferably the first 30 days, preferably the first 31 days, preferably the first 32 days, preferably the first 33 days, preferably the first 34 days, preferably the first 35 days, preferably the first 36 days, preferably the first 37 days, preferably the first 38 days, preferably the first 39 days, preferably the first 40 days or more followed by a lower and/or a higher dose (e.g., biweekly dose, weekly dose, twice weekly dose, daily dose) for the remaining days of the same phase. For instance, any of dose (A) or dose (B) may be unchanged for the first 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 days or more followed by a lower and/or a higher dose (e.g., biweekly dose, weekly dose, twice weekly dose, daily dose) for the remaining days of the same phase. Said remaining days of each phase can be 1 day, preferably 2 days, preferably 3 days, preferably 4 days, preferably 5 days, preferably 6 days, preferably 7 days, preferably 8 days, preferably 9 days, preferably 10 days, preferably 11 days, preferably 12 days, preferably 13 days, preferably 14 days, preferably 15 days, preferably 16 days, preferably 17 days, preferably 18 days, preferably 19 days, preferably 20 days, preferably 21 days, preferably 22 days, preferably 23 days, preferably 24 days, preferably 25 days, preferably 26 days, preferably 27 days, preferably 28 days, preferably 29 days, preferably 30 days, preferably 31 days, preferably 32 days, preferably 33 days, preferably 34 days, preferably 35 days, preferably 36 days, preferably 37 days, preferably 38 days, preferably 39 days, preferably 40 days, preferably 41 days, preferably 42 days, preferably 43 days, preferably 44 days, preferably 45 days, preferably 46 days, preferably 47 days, preferably 48 days, preferably 49 days, preferably 50 days, preferably 51 days, preferably 52 days, preferably 53 days, preferably 54 days, preferably 55 days, preferably 56 days, preferably 57 days, preferably 58 days, preferably 59 days, preferably 60 days or more. Said remaining days of each phase can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more. The pharmaceutical formulation and/or the oral dosage form may also be administered at least once every week, preferably at least twice every week, preferably at least once every two weeks, preferably at least once every three weeks, preferably at least once every four weeks, preferably at least once a month, preferably at least once every two months, preferably at least once every three months, preferably at least once every four months, preferably at least once every five months, preferably at least once every sixth months, preferably at least once a year. The pharmaceutical formulation and/or the oral dosage form may also be administered daily for 1 day, preferably 2 days, preferably 3 days, preferably 4 days, preferably 5 days, preferably 6 days, preferably 7 days, preferably 8 days, preferably 9 days, preferably 10 days, preferably 11 days, preferably 12 days, preferably 13 days, preferably 14 days, preferably 15 days, preferably 16 days, preferably 17 days, preferably 18 days, preferably 19 days, preferably 20 days, preferably 21 days, preferably 22 days, preferably 23 days, preferably 24 days, preferably 25 days, preferably 26 days, preferably 27 days, preferably 28 days, preferably 29 days, preferably 30 days, preferably 31 days, preferably 32 days, preferably 33 days, preferably 34 days, preferably 35 days, preferably 36 days, preferably 37 days, preferably 38 days, preferably 39 days, preferably 40 days, preferably 41 days, preferably 42 days, preferably 43 days, preferably 44 days, preferably 45 days, preferably 46 days, preferably 47 days, preferably 48 days, preferably 49 days, preferably 50 days, preferably 51 days, preferably 52 days, preferably 53 days, preferably 54 days, preferably 55 days, preferably 56 days, preferably 57 days, preferably 58 days, preferably 59 days, preferably 60 days or more followed by at least an administration at least once every week, preferably at least twice every week, preferably at least once every two weeks, preferably at least once every three weeks, preferably at least once every four weeks or a month, preferably at least once every two months, preferably at least once every three months, preferably at least once every four months, preferably at least once every five months, preferably at least once every sixth months, preferably at least once a year. The pharmaceutical formulation and/or the oral dosage form may also be administered daily for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more followed by at least an administration at least once every week, at least twice every week, at least once every two weeks, at least once every three weeks, at least once every four weeks or a month, at least once every two months, at least once every three months, at least once every four months, at least once every five months, at least once every sixth months or at least once a year.

In some embodiments, the pharmaceutical formulation according to the invention comprises a crystallization rate inhibitor. The term “crystallization rate inhibitor” refers to an excipient, for example a polymeric excipient, that is added to the formulation with the aim of inhibiting crystallization of an API when the formulation is administered to a subject. A crystallization rate inhibitor may be used to improve the bioavailability of an API where the bioavailability of the crystalline form is significantly lower in comparison to the amorphous/dissolved state. The crystallization rate inhibitor may be referred to as a crystallization inhibitor or a stabilizer.

In an embodiment, the crystallization rate inhibitor is selected from polyvinylpyrrolidone (PVP), a polyvinylpyrrolidone-vinyl acetate copolymer (PVPVA), a poly(meth)acrylate polymer (e.g. methacrylic acid-methyl methacrylate copolymer), a cyclodextrin or a cyclodextrin derivative (e.g. (2-hydroxypropyl)-β-cyclodextrin (HPBCD)), hydroxypropylcellulose, hydroxyethylcellulose, methylcellulose, hydroxypropyl methylcellulose (HPMC), hydroxypropyl methylcellulose acetate succinate (HPMCAS), a polyethylene glycol-polyvinyl acetate-polyvinyl caprolactame graft copolymer, poly(vinyl alcohol), a poloxamer (e.g. poloxamer 188, 338, or 407), and any combinations thereof.

In an embodiment, the crystallization rate inhibitor is selected from hydroxypropyl methylcellulose (HPMC), hydroxypropyl methylcellulose acetate succinate (HPMCAS), a polyethylene glycol-polyvinyl acetate-polyvinyl caprolactame graft copolymer, polyvinylpyrrolidone (PVP) and a polyvinylpyrrolidone-vinyl acetate copolymer (PVPVA), and a combination thereof. In a further embodiment, the crystallization rate inhibitor is selected from hydroxypropyl methylcellulose (HPMC) and polyvinylpyrrolidone-vinyl acetate copolymer (PVPVA). The PVPVA may be a copolymer of 1-vinyl-2-pyrrolidone and vinyl acetate in a ratio of 6:4 by mass (PVPVA64).

Names and abbreviations for polyvinylpyrrolidone-vinyl acetate copolymer include, but are not limited to, PVPVA, PVP-Vac-copolymer, and poly(1-vinylpyrrolidone-co-vinylacetate).

Names and abbreviations for a copolymer of 1-vinyl-2-pyrrolidone and vinyl acetate in a ratio of 6:4 by mass (PVPVA64) include, but are not limited to, copolyvidone, copovidum, and copovidone. Examples of commercially available PVPVA64 are Kollidon® VA64, Kollidon® VA64 Fine, Luviskol VA64®, and Plasdone S-630®.

Names and abbreviations for polyvinylpyrrolidone include, but are not limited to, PVP, povidone and crospovidone. Crospovidone is a crosslinked homopolymer of vinyl pyrrolidone. An example of commercially available PVP is Plasdone® K-12.

Hydroxypropyl methylcellulose, also known as Hypromellose (HPMC) is an anhydroglucose in which some of the hydroxyl groups are substituted with methyl groups to form methyl ether moieties, and others are substituted with hydroxypropyl groups or with methoxypropyl groups to form hydroxypropyl ether or methoxypropyl ether moieties.

Hydroxypropyl methylcellulose polymers (HPMCs) are available in different viscosity grades from several sources such as Dow Chemical Co. under the brand name Methocel® and from Shin Etsu under Metolose®. Non-limiting examples of low viscosity polymers are Methocel E5®, Methocel E6®, Methocel E-15LV®, Methocel E50LV®, Methocel K100LVR and Methocel F50LV®, whose 2% aqueous solutions at 25° C. have viscosities of about 5 mPas, 6 mPas, 15 mPas, 50 mPas, 100 mPas and 50 mPas, respectively. Non-limiting examples of medium viscosity HPMCs are Methocel E4M® and Methocel K4M, whose 2% aqueous solutions at 25° C. have viscosities of 4,000 mPas. Non-limiting examples of high viscosity HPMCs are Methocel K15M® and Methocel K100M® whose 2% aqueous solutions at 25° C. have viscosities of 15,000 mPas and 100,000 mPas.

In some embodiments, the hydroxypropyl methylcellulose has a viscosity ranging between 3 and 5000 mPa·s (2% in H₂O at 25° C.) and can be selected from the group comprising HPMC E5, HPMC E6, HPMC E15, HPMC E50, HPMC K4M, HPMC K15M, HPMC-AS and mixtures thereof. In some embodiments, the hydroxypropyl methylcellulose has a viscosity ranging between 3 and 500 mPa·s (2% in H₂O at 25° C.) and can be selected from the group comprising HPMC E5, HPMC E6, HPMC E15, HPMC E50, and mixtures thereof. In some embodiments, the hydroxypropyl methylcellulose has a viscosity ranging between 3 and 50 mPa·s (2% in H₂O at 25° C.) and can be selected from the group comprising HPMC E5, HPMC E6, HPMC E15, HPMC E50, and mixtures thereof.

As used herein the term “methacrylic acid copolymer”, preferably refers to a copolymer of acrylic- and/or methacrylic acids/ester such as those compounds sold under the trade name Eudragit®. Eudragit® is commercially available, for example, from Evonik Healthcare & Nutrition GmbH, Essen, Germany.

Different types of methacrylic acid copolymer can be used, and they include poly(methacrylic acid co-methyl methacrylate) 1:1 (such as Eudragit® L-100, Eudragit® L12.5); poly(methacrylic acid co-methyl methacrylate) 1:2 (such as Eudragit® S-100, Eudragit® S12,5, Eudragit® FS30D); poly(methacrylic acid co-ethyl acrylate) 1:1 (such as Eudragit® L30D55, Eudragit® L100-55); Poly(ethyl acrylate-co-methyl methacrylate-co trimethylammonioethyl methacrylate chloride) 1:2:0.1 (such as Eudragit® RS30D); poly(ethyl acrylate, methyl methacrylate, trimethylammonioethyl methacrylate chloride) 1:2:0.2 (such as Eudragit® RL30D); poly(ethyl acrylate co-methyl methacrylate) 2:1 (such as Eudragit® NM 30D, or Eudragit NE 30D); cationic copolymer based on dimethylaminoethyl methacrylate, butyl methacrylate, and methyl methacrylate with a ratio of 2:1:1 (Eudragit® E-100) and combinations thereof.

In some embodiments, the methacrylic acid copolymer is selected from the group comprising a copolymer of methacrylic acid and methyl methacrylate; a copolymer of methacrylic acid and ethyl acrylate; and mixture thereof.

In some embodiments, the methacrylic acid copolymer is a copolymer of methacrylic acid and methyl methacrylate. Preferably, the ratio of methacrylic acid to methyl methacrylate in the copolymer is 0.5:2 to 2:0.5, preferably 0.8:1 to 1.2:1 (e.g., 1:1).

In some embodiments, the methacrylic acid copolymer is poly(methacrylic acid-co-methyl methacrylate) 1:1 (EUDRAGIT® L100, CAS number: 25086-15-1).

In some embodiments, oral dosage form and/or pharmaceutical formulation according to the present invention comprises, a) a compound of formula (I) as defined herein above and preferably

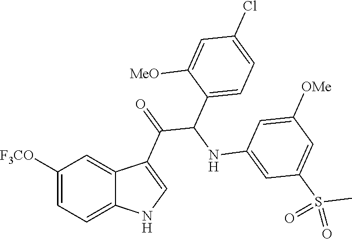 [SMILES: COc1cc(NC(C(=O)c2c[nH]c3ccc(OC(F)(F)F)cc23)c2ccc(Cl)cc2OC)cc(S(C)(=O)=O)c1]

or a stereo-isomeric form, a pharmaceutically acceptable salt, solvate or polymorph thereof; and
b1) methacrylic acid copolymer, or
b2) a cellulose derivative such as hydroxypropyl methylcellulose (HPMC).

The oral dosage form and/or pharmaceutical formulation of the invention may comprise at most 50 wt % of compound of formula (I) relative to the total weight of the formulation, preferably at most 40 wt %, preferably at most 35 wt %, preferably at most 30 wt %, preferably at most 25 wt % of compound of formula (I) relative to the total weight of the formulation. The pharmaceutical formulation may comprise at least 0.1 wt % of compound of formula (I) relative to the total weight of the formulation, preferably at least 0.5 wt %, preferably at least 1 wt %, preferably at least 5 wt %, preferably at least 10 wt %, preferably at least 15 wt %, preferably at least 17 wt %, preferably or at least 20 wt % of compound of formula (I) relative to the total weight of the formulation. The pharmaceutical formulation may comprise from 0.1 wt % to 45 wt % of compound of formula (I) relative to the total weight of the formulation, preferably from 0.5 wt % to 40 wt %, preferably from 1 wt % to 40 wt %, preferably from 5 wt % to 35 wt %, preferably from 10 wt % to 35 wt % of compound of formula (I) relative to the total weight of the formulation.

The oral dosage form and/or pharmaceutical formulation of the invention may contain from 0.1 mg to 3000 mg of the compound of formula (I), preferably from 1 mg to 2000 mg of compound of formula (I), preferably from 5 mg to 1500 mg of compound of formula (I), preferably from 5 to 1000 mg of compound of formula (I), preferably from 10 to 1100 mg of compound of formula (I), preferably from 15 to 950 mg of the compound of formula (I), preferably from 20 to 900 mg of compound of formula (I) or any particular amount or range comprised therein.

The oral dosage form and/or pharmaceutical formulation of the invention may comprise:
from 20 mg to 6000 mg of the of the methacrylic acid copolymer, preferably from 30 mg to 4000 mg of the methacrylic acid copolymer, preferably from 40 mg to 2000 mg of the methacrylic acid copolymer, preferably from 50 mg to 1500 mg of the methacrylic acid copolymer, preferably from 60 mg to 1000 mg of the methacrylic acid copolymer, preferably from 70 mg to 1000 mg of the methacrylic acid copolymer, preferably from 80 mg to 600 mg of the methacrylic acid copolymer, or any particular amount or range comprised therein; or
from 20 mg to 6000 mg of the of the hydroxypropyl methylcellulose, preferably from 30 mg to 4000 mg of the hydroxypropyl methylcellulose, preferably from 40 mg to 2000 mg of the hydroxypropyl methylcellulose, preferably from 50 mg to 1500 mg of the hydroxypropyl methylcellulose, preferably from 60 mg to 1000 mg of the hydroxypropyl methylcellulose, preferably from 70 mg to 1000 mg of the hydroxypropyl methylcellulose, preferably from 80 mg to 600 mg of the hydroxypropyl methylcellulose or, any particular amount or range comprised therein.

The oral dosage form and/or pharmaceutical formulation of the invention may further comprise one or more diluents; wherein the formulation comprises from 20 mg to 7500 mg of the diluent, preferably from 30 mg to 6500 mg, preferably from 40 mg to 4500 mg, preferably from 50 mg to 2500 mg, preferably from 60 mg to 2000 mg, preferably from 80 mg to 1000 mg, preferably from 90 mg to 550 mg of the diluent, or any particular amount or range comprised therein.

The oral dosage form and/or pharmaceutical formulation of the invention may further comprise one or more surfactants; wherein the formulation comprises from 0.5 mg to 300 mg of the surfactant, preferably from 0.6 mg to 250 mg, preferably from 0.8 mg to 200 mg, preferably from 1 mg to 150 mg, preferably from 1.2 mg to 100 mg, preferably from 1.5 mg to 80 mg, preferably from 2 mg to 30 mg of the surfactant, or any particular amount or range comprised therein.

The oral dosage form and/or pharmaceutical formulation of the invention may further comprise one or more disintegrant; wherein the formulation comprises from 3 mg to 900 mg of the disintegrant, preferably from 4 mg to 850 mg, preferably from 5 mg to 600 mg, preferably from 6 mg to 500 mg, preferably from 7 mg to 400 mg, preferably from 7.5 mg to 200 mg, preferably from 8 mg to 100 mg of the disintegrant, or any particular amount or range comprised therein.

The oral dosage form and/or pharmaceutical formulation of the invention may further comprise one or more glidant; wherein the formulation comprises from 1 mg to 400 mg of the glidant, preferably from 2 mg to 350 mg, preferably from 3 mg to 300 mg, preferably from 4 mg to 200 mg, preferably from 5 mg to 100 mg, preferably from 6 mg to 50 mg, preferably from 8.5 mg to 35 mg of the glidant, or any particular amount or range comprised therein.

The oral dosage form and/or pharmaceutical formulation of the invention may further comprise one or more lubricant; wherein the formulation comprises from 0.5 mg to 200 mg of the lubricant, preferably from 1 mg to 150 mg, preferably from 3 mg to 100 mg, preferably from 4 mg to 90 mg, preferably from 7 mg to 90 mg, preferably from 8 mg to 80 mg, preferably from 10 mg to 50 mg of the lubricant, or any particular amount or range comprised therein.

The oral dosage form and/or pharmaceutical formulation of the invention may comprise:
at most 60 wt %, preferably most 50 wt %, preferably at most 45 wt %, preferably at most 40 wt %, preferably at most 35 wt % of the methacrylic acid copolymer relative to the total weight of the formulation; or
at most 60 wt %, preferably most 50 wt %, preferably at most 45 wt %, preferably at most 40 wt %, preferably at most 35 wt % of the hydroxypropyl methylcellulose relative to the total weight of the formulation.

The oral dosage form and/or pharmaceutical formulation of the invention may comprise:
at least 0.2 wt %, preferably at least 1 wt %, preferably at least 5 wt %, preferably at least 10 wt %, preferably at least 20 wt %, of the methacrylic acid copolymer relative to the total weight of the formulation; or
at least 0.2 wt %, preferably at least 1 wt %, preferably at least 5 wt %, preferably at least 10 wt %, preferably at least 20 wt %, of the hydroxypropyl methylcellulose relative to the total weight of the formulation.

The oral dosage form and/or pharmaceutical formulation according to the present invention may comprise:
from 0.2 wt % to 60 wt %, preferably from 1 wt % to 50 wt %, preferably from 5 wt % to 40 wt % of the methacrylic acid copolymer relative to the total weight of the formulation; or
from 0.2 wt % to 60 wt %, preferably from 1 wt % to 50 wt %, preferably from 5 wt % to 40 wt % of the hydroxypropyl methylcellulose relative to the total weight of the formulation.

In some embodiments, the compound of formula (I) and the methacrylic acid copolymer are present in the oral dosage form and/or pharmaceutical formulation of the invention in a ratio of 4:1 w/w; preferably a ratio of 3.8:1 w/w; preferably a ratio of 3.5:1 w/w; preferably a ratio of 3.3:1 w/w; preferably a ratio of 3:1 w/w; preferably a ratio of 2.8:1 w/w; preferably a ratio of 2.5:1 w/w; preferably a ratio of 2.3:1 w/w; preferably a ratio of 2:1 w/w; preferably a ratio of 1.8:1 w/w; preferably a ratio of 1.5:1 w/w; preferably a ratio of 1:5 w/w; preferably a ratio of 1:4.8 w/w; preferably a ratio of 1:4.5 w/w; preferably a ratio of 1:4.3 w/w; preferably a ratio of 1:4 w/w; preferably a ratio of 1:3.8 w/w; preferably a ratio of 1:3.5 w/w; preferably a ratio of 1:3.3 w/w; preferably a ratio of 1:3 w/w; preferably a ratio of 1:2.8 w/w; preferably a ratio of 1:2.5 w/w; preferably a ratio of 1:2.3 w/w; preferably a ratio of 1:2 w/w; preferably a ratio of 1:1.5 w/w, preferably a ratio of 1:1.2 w/w or a ratio comprised within any two ratios mentioned herein, or a ratio range or sub-range within any two ratios mentioned herein.

In some embodiments, the compound of formula (I) and the hydroxypropyl methylcellulose are present in the oral dosage form and/or pharmaceutical formulation of the invention in a ratio of 4:1 w/w compound of formula (I): hydroxypropyl methylcellulose; preferably a ratio of 3.8:1 w/w compound of formula (I): hydroxypropyl methylcellulose; preferably a ratio of 3.5:1 w/w compound of formula (I): hydroxypropyl methylcellulose; preferably a ratio of 3.3:1 w/w compound of formula (I): hydroxypropyl methylcellulose; preferably a ratio of 3:1 w/w compound of formula (I): hydroxypropyl methylcellulose; preferably a ratio of 2.8:1 w/w compound of formula (I): hydroxypropyl methylcellulose; preferably a ratio of 2.5:1 w/w compound of formula (I): hydroxypropyl methylcellulose; preferably a ratio of 2.3:1 w/w compound of formula (I): hydroxypropyl methylcellulose; preferably a ratio of 2:1 w/w compound of formula (I): hydroxypropyl methylcellulose; preferably a ratio of 1.8:1 w/w compound of formula (I): hydroxypropyl methylcellulose; preferably a ratio of 1.5:1 w/w compound of formula (I): hydroxypropyl methylcellulose; preferably a ratio of 1:5 w/w compound of formula (I): hydroxypropyl methylcellulose; preferably a ratio of 1:4.8 w/w compound of formula (I): hydroxypropyl methylcellulose; preferably a ratio of 1:4.5 w/w compound of formula (I): hydroxypropyl methylcellulose; preferably a ratio of 1:4.3 w/w compound of formula (I): hydroxypropyl methylcellulose; preferably a ratio of 1:4 w/w compound of formula (I): hydroxypropyl methylcellulose; preferably a ratio of 1:3.8 w/w compound of formula (I): hydroxypropyl methylcellulose; preferably a ratio of 1:3.5 w/w compound of formula (I): hydroxypropyl methylcellulose; preferably a ratio of 1:3.3 w/w compound of formula (I): hydroxypropyl methylcellulose; preferably a ratio of 1:3 w/w compound of formula (I): hydroxypropyl methylcellulose; preferably a ratio of 1:2.8 w/w compound of formula (I): hydroxypropyl methylcellulose; preferably a ratio of 1:2.5 w/w compound of formula (I): hydroxypropyl methylcellulose; preferably a ratio of 1:2.3 w/w compound of formula (I): hydroxypropyl methylcellulose; preferably a ratio of 1:2 w/w compound of formula (I): hydroxypropyl methylcellulose; preferably a ratio of 1:1.5 w/w compound of formula (I): hydroxypropyl methylcellulose, preferably a ratio of 1:1.2 w/w compound of formula (I): hydroxypropyl methylcellulose or a ratio comprised within any two ratios mentioned herein, or a ratio range or sub-range within any two ratios mentioned herein.

The oral dosage form and/or pharmaceutical formulation of the invention may further comprise one or more pharmaceutically acceptable excipients, as described in more detail herein. Pharmaceutically acceptable excipients include, but are not limited to, disintegrants, binders, diluents, lubricants, stabilizers, osmotic agents, colorants, plasticizers, coatings, fillers, surfactants and the like. Additional suitable pharmaceutical excipients and their properties may be found in texts such as Handbook of Pharmaceutical Excipients, Edited by R. C. Rowe, P. J. Sheskey & P. J. Weller, Sixth Edition (Published by Pharmaceutical Press, a Division of Royal Pharmaceutical Society of Great Britain).

Diluents (or fillers) useful in the present invention include microcrystalline celluloses (e.g., Avicel® PH 102, Avicel® PH 101, Ceolus UF, Ceolus KG, or Ceolus PH), silicified microcrystalline celluloses, lactoses, sorbitols, celluloses, calcium phosphates, starches (e.g., partially or fully pregelatinized maize starch), sugars or lactoses (e.g., mannitol, sucrose, or the like), or any combination thereof. Non-limiting examples of microcrystalline celluloses include commercially available Avicel® series, such as microcrystalline celluloses having a particle size of 100 μm (e.g., Avicel® PH 102). Microcrystalline celluloses also include commercially available Ceolus in UF, KG, or PH grade. Other non-limiting examples of diluents include silicified microcrystalline celluloses, such as commercially available Prosolv® series (e.g., Prosolv® SMCC 50 and SMCC HD90). Lactoses suitable for the invention includes lactose monohydrate. Amounts of the diluents relative to the total weight of the pharmaceutical formulation may be 5 wt % to 95 wt %, preferably 20 wt % to 80 wt %, preferably 25 wt % to 50 wt %, preferably 30 wt % to 48 wt %, preferably 30 wt % to 52 wt %, preferably 35 wt % to 52 wt %, preferably 40 wt % to 50 wt %. For example, the diluent in the pharmaceutical formulation may comprise microcrystalline cellulose, silicified microcrystalline cellulose, and partially or fully pregelatinized maize starch having a combined (or total) concentration of 5 wt % to 95 wt %, preferably 20 wt % to 80 wt %, preferably 25 wt % to 50 wt %, preferably 30 wt % to 48 wt %, preferably 30 wt % to 52 wt %, preferably 35 wt % to 52 wt %, preferably 30 wt % to 45 wt %, preferably 32.5 wt % to 45 wt % by weight of the pharmaceutical formulation.

Disintegrants enhance the dispersal of pharmaceutical formulations. Non-limiting examples of disintegrants that are useful in the present invention include crosscarmellose (e.g., croscarmellose sodium), crospovidone, metal starch glycolates (e.g., sodium starch glycolate), and any combination thereof. Other examples of disintegrants include croscarmellose sodium (e.g., Ac-Di-Sol®) and sodium starch glycolate. Pharmaceutical formulations of the present invention may comprise one or more disintegrants giving a combined (or total) concentration of 1 wt % to 10 wt. %, preferably 5 wt % to 9 wt. %, preferably 6 wt % to 8 wt. %, preferably 6.5 wt % to 7.5 wt. %, preferably 6.75 wt % to 7.25 wt. %, preferably 3 wt % to 7 wt. %, preferably 1 wt % to 7 wt. %, or preferably 1.2 wt % to 8.2 wt % of the pharmaceutical formulation. In some embodiments, the pharmaceutical formulation comprises 2 wt % to 8 wt % (e.g., 2.5 wt % to 7.5 wt. %), preferably from 3 wt % to 6 wt %; more preferably from 4 wt % to 5 wt % of disintegrant (e.g., crospovidone) by weight of the pharmaceutical formulation.

Binders may include agents used while making granules of the active pharmaceutical ingredient by mixing the binder(s) with diluent and the active pharmaceutical ingredient. Non-limiting examples of binders useful in the present invention include polyvinyl pyrrolidones, sugar, modified celluloses (e.g., hydroxypropyl methylcelluloses (HPMC), hydroxy propyl celluloses (HPC), and hydroxy ethyl celluloses (HEC)), and any combination thereof. Other examples of the binders include polyvinyl pyrrolidones (PVP). An example of HPC includes a low viscosity polymer, HPC-SL. PVP may be characterized by its “K-value”, which is a useful measure of the polymeric composition's viscosity. PVP can be commercially purchased (e.g., Tokyo Chemical Industry Co., Ltd.) under the trade name of Povidone® K12, Povidone® K17, Povidone® K25, Povidone® K30, Povidone® K60, and Povidone® K90. Specific examples of PVP include soluble spray dried PVP. Another example includes PVP having an average molecular weight of 3,000 to 4,000, such as Povidone® K12 having an average molecular weight of 4,000. PVP can be used in either wet or dry state. Pharmaceutical formulations of the present invention may comprise one or more binders giving a combined (or total) concentration of 0.1 wt % to 50 wt %; preferably 0.5 wt % to 43 wt %; preferably 2 wt % to 45 wt %; preferably 5 wt % to 40 wt %, preferably 10 wt % to 35 wt %, preferably 15 wt % to 30 wt %; preferably 20 wt % to 25 wt % of the pharmaceutical formulation. In some embodiments, the pharmaceutical formulation comprises 0.5 wt % to 2 wt % (e.g., 1.5 wt % to 2 wt % or 1.75 wt % to 2.25 wt %) of binder (e.g., hydroxypropyl methylcellulose) by weight of the pharmaceutical formulation.

Lubricants function to improve the compression and ejection of pharmaceutical formulations from, e.g., a die press. Non-limiting examples of lubricants useful in the present invention include magnesium stearate, stearic acid (stearin), hydrogenated oil, sodium stearyl fumarate, compritol (glyceryl behenate) and any combination thereof. In one example, the lubricant includes sodium stearyl fumarate. In another example, the lubricant includes magnesium stearate. Pharmaceutical formulations of the present invention may comprise one or more lubricants giving a combined (or total) concentration of 0.10 wt % to 10 wt %, preferably 0.5 wt % to 6 wt %, preferably 0.8 wt % to 3.5 wt %, preferably 1 wt % to 3 wt %, preferably 1.50 wt % to 5.5 wt %, preferably 2 wt % to 4 wt % or preferably 0.25 wt % to 5.25 wt % by weight of the pharmaceutical formulation. In some embodiments, the pharmaceutical formulation comprises 0.5 wt % to 5.5 wt %, preferably 1 wt % to 2.5 wt % of lubricant (e.g., magnesium stearate).

One or more wetting agents can be employed in the oral dosage form and/or pharmaceutical formulations of the invention. Wetting agents suitable for the present invention generally enhance the solubility of pharmaceutical formulations. Wetting agents include surfactants, such as non-ionic surfactants and anionic surfactants. Non-limiting examples of surfactants useful in the invention include sodium lauryl sulfate (SLS), polyoxyethylene sorbitan fatty acids (e.g., polysorbate 20 (e.g., TWEEN 20™)), sorbitan fatty acid esters (e.g., Spans®), sodium dodecylbenzene sulfonate (SDBS), dioctyl sodium sulfosuccinate (Docusate), dioxycholic acid sodium salt (DOSS), sorbitan monostearate, sorbitan tristearate, sodium N-lauroylsarcosine, sodium oleate, sodium myristate, sodium stearate, sodium palmitate, gelucire 44/14, ethylenediamine tetraacetic acid (EDTA), vitamin E d-alpha tocopheryl polyethylene glycol 1000 succinate (TPGS), lecithin, 15 MW 677-692, glutanic acid monosodium monohydrate, labrasol, PEG 8 caprylic/capric glycerides, transcutol, diethylene glycol monoethyl ether, solutol HS-15, Soluplus®, polyethylene glycol/hydroxystearate, taurocholic acid, copolymers of polyoxypropylene and polyoxyethylene (e.g., poloxamers also known and commercially available under Pluronics®, such as, Pluronic® L61, Pluronic® F68, Pluronic® F108, and Pluronic® F127), saturated polyglycolized glycerides (Gelucirs®), and any combination thereof. Other examples include sodium lauryl sulfate, which is an anionic surfactant; and copolymers of polyoxypropylene and polyoxyethylene which are non-ionic surfactants. Examples of the copolymers of polyoxypropylene and polyoxyethylene include poloxamers, such as poloxamer with a polyoxypropylene molecular mass of 1,800 g/mol and an 80% polyoxyethylene content (e.g., poloxamer 188). Pharmaceutical formulations of the present invention may comprise one or more wetting agents giving a combined (or total) concentration of 0.25 wt % to 10 wt. %, preferably 0.25 wt % to 5.75 wt. %, preferably 0.50 wt % to 5 wt. %, preferably 1 wt % to 3 wt % or preferably 1.50 wt % to 2 wt % by weight of the pharmaceutical formulation. In some embodiments, the pharmaceutical formulation comprises 0.25 wt % to 5.00 wt % (e.g., 0.35 wt % to 4.50 wt. %) of a wetting agent (e.g., sodium lauryl sulfate).

Glidants enhance the flow properties of formulations during processing into final drug product form. Non-limiting examples of glidants useful in the present invention include silicon dioxide (e.g., colloidal fumed silica, colloidal anhydrous silica) and/or talc. Specific examples of glidants include colloidal fumed silica (e.g., Aerosil® 200) Pharmaceutical formulations of the present invention may comprise one or more glidants giving a combined (or total) concentration of 0.10 wt % to 10 wt %, preferably 1 wt % to 8 wt %; preferably 2 wt % to 7.5 wt %; preferably 3 wt % to 5 wt % by the weight of the pharmaceutical formulation. In some embodiments, the pharmaceutical formulation comprises glidant in an amount of 0.10 wt % to 5 wt % (e.g., 0.75 wt % to 3.25 wt. %) by weight of the pharmaceutical formulation. In other embodiments, the pharmaceutical formulation comprises fumed silica in an amount of 0.10 wt % to 2 wt % (e.g., 0.75 wt % to 1.5 wt. %) by weight of the pharmaceutical formulation.

In some embodiments the oral dosage form according to the invention is a solid dosage form formulated in a pharmaceutical formulation; preferably the solid dosage form is a tablet. Tablet dosage forms of the present invention may further comprise a coating. Suitable coatings are film-forming polymers, such as, for example, those from the group of the cellulose derivatives (such as HPC (hydroxypropylcellulose), HPMC (hydroxypropoxymethylcellulose), MC (methylcellulose), HPMCAS (hydroxypropoxymethylcelluclose acetate succinate), dextrins, starches, natural gums, such as, for example, gum arabic, xanthans, alginates, polyvinyl alcohol, polymethacrylates and derivatives thereof, such as, for example, Eudragit®, which may be applied to the tablet as solutions or suspensions by means of the various pharmaceutical conventional methods, such as, for example, film coating. The coating is typically applied as a solution/suspension which, in addition to any film-forming polymer present, may further comprise one or more adjuvants, such as hydrophilizers, plasticizers, surfactants, dyes and white pigments, such as, for example, titanium dioxide.

In some embodiments the oral dosage form according to the invention is formulated in a pharmaceutical formulation comprising a plurality of granules forming an intragranular phase of the formulation and one or more pharmaceutically acceptable excipients forming an extragranular phase of the formulation. Preferably the oral dosage form is a tablet, said tablet comprising an intragranular phase and an extragranular phase.

As used herein, the term “intragranular phase” refers to those components of a formulation that are with granules. As used herein, the term “extragranular phase” refers to those components of a formulation that are outside of the granules.

In some embodiments the intragranular phase of the pharmaceutical formulation according to the invention comprises the active pharmaceutical ingredient and one of or a combination of methacrylic acid copolymer, or a cellulose derivative such as methyl cellulose (MC), ethyl cellulose (EC), hydroxyethyl cellulose (HEC), hydroxypropyl cellulose (HPC), carboxymethyl cellulose (CMC), sodium carboxymethyl cellulose (NaCMC) or hydroxypropyl methylcellulose (HPMC). In some embodiments the intragranular phase of the pharmaceutical formulation according to the invention comprises the active pharmaceutical ingredient and one of or a combination of methacrylic acid copolymer, or hydroxypropyl methylcellulose (HPMC) and one or more pharmaceutically acceptable excipient selected from disintegrants, binders, diluents, lubricants, stabilizers, wetting agents, glidants, osmotic agents, colorants, plasticizers, coatings, fillers and surfactants.

In some embodiments the pharmaceutical formulation comprises at least 15 wt %; at least 20 wt %; preferably at least 25 wt %; preferably at least 28 wt %; preferably at least 30 wt %; preferably at least 34 wt %; preferably at least 40 wt %; preferably at least 45 wt %, preferably at least 50 wt %; preferably at least 55 wt %; preferably at least 60 wt %; preferably at least 65 wt % of intragranular phase by the total weight of the pharmaceutical formulation. In some embodiments the pharmaceutical formulation comprises at most 99 wt %; preferably at most 95 wt %; preferably at most 93 wt %; preferably at most 90 wt %; preferably at most 85 wt %; preferably at most 80 wt %; preferably at most 75 wt %; preferably at most 74 wt %; preferably at most 73 wt %; preferably at most 70 wt %; preferably at most 67 wt %; preferably at most 63 wt %; preferably at most 60% wt %; preferably at most 53 wt % of intragranular phase by the total weight of the pharmaceutical formulation.

In some embodiments the pharmaceutical formulation comprises an intragranular phase comprising from preferably from 1 wt % to 70 wt %; preferably from 2 wt % to 69 wt %; preferably from 3 wt % to 68 wt %; preferably from 4 wt % to 67 wt %; preferably from 5 wt % to 66 wt %; preferably from 6 wt % to 65 wt %; preferably from 10 wt % to 64 wt %; preferably from 15 wt % to 60 wt %; preferably from 20 wt % to 55 wt %; preferably from 25 wt % to 50 wt %; preferably from 30 wt % to 45 wt %; preferably from 35 to 40 wt % of API by the total weight of the pharmaceutical formulation.

In some embodiments the oral dosage form is formulated in a pharmaceutical formulation comprises an intragranular phase comprising from 5 wt % to 60 wt %; preferably from 7 wt % to 55 wt %; preferably from 8 wt % to 50 wt %; preferably from 17 wt % to 45 wt %; preferably from 15 wt % to 45 wt % of methacrylic acid copolymer or hydroxypropyl methylcellulose or a combination thereof by the total weight of the pharmaceutical formulation.

In some embodiments the oral dosage form is formulated in a pharmaceutical formulation comprises an intragranular phase comprising from 5 wt % to 60 wt %; preferably from 10 wt % to 60 wt %; preferably from 12 wt % to 60 wt %; preferably from 15 wt % to 50 wt %; preferably from 18 wt % to 45 wt % of filler by the total weight of the pharmaceutical formulation.

In some embodiments the oral dosage form is formulated in a pharmaceutical formulation comprises an intragranular phase comprising from 1 wt % to 10 wt %; preferably from 1 wt % to 9 wt %, preferably from 1.7 wt % to 8 wt %; preferably from 1.6 wt % to 7.5 wt %; preferably from 2 wt % to 5 wt % of disintegrant by the total weight of the pharmaceutical formulation.

In some embodiments the oral dosage form is formulated in a pharmaceutical formulation comprises an intragranular phase comprising from 0.1 wt % to 5 wt %; preferably from 0.2 wt % to 4.7 wt %; preferably from 0.5 wt % to 4.5 wt %; preferably from 0.8 wt % to 4 wt %; preferably from 1 wt % to 3.5 wt % of glidant by the total weight of the pharmaceutical formulation.

In some embodiments the oral dosage form is formulated in a pharmaceutical formulation comprises an intragranular phase comprising from 0.1 wt % to 5 wt %; preferably from 0.2 wt % to 4.7 wt %; preferably from 0.5 wt % to 4.5 wt %; preferably from 0.6 wt % to 4.5 wt %; preferably from 0.8 wt % to 4.0 wt %; preferably from 1 wt % to 3.5 wt % of surfactant by the total weight of the pharmaceutical formulation.

In some embodiments the oral dosage form is formulated in a pharmaceutical formulation comprises an intragranular phase comprising from 0.1 wt % to 3 wt %; preferably from 0.2 wt % to 2.7 wt %; preferably from 0.5 wt % to 2.5 wt %; preferably from 0.7 wt % to 2 wt % of lubricant by the total weight of the pharmaceutical formulation.

In some embodiments the oral dosage form is formulated in a pharmaceutical formulation comprising at least 5 wt %; preferably at least 7 wt %; preferably at least 10 wt %; preferably at least 15 wt %; preferably at least 20 wt %; preferably at least 25 wt %; preferably at least 28 wt %; preferably at least 30 wt %; preferably at least 34 wt %; preferably at least 35 wt % of extragranular phase by the total weight of the pharmaceutical formulation. In some embodiments the pharmaceutical formulation comprises at most 75 wt %; preferably at most 74 wt %; preferably at most 73 wt %; preferably at most 70 wt %; preferably at most 67 wt %; preferably at most 63 wt %; preferably at most 65 wt %; preferably at most 60 wt %; preferably at most 55 wt %, preferably at most 53 wt %, preferably at most 40 wt % of extragranular phase by the total weight of the pharmaceutical formulation. In some embodiments the pharmaceutical formulation comprises at least 15 wt % to at most 75 wt %; preferably at least 25 wt % to at most 70 wt %; preferably at least 30 wt % to at most 65 wt %; preferably at least 35 wt % to at most 60 wt %; preferably at least 35 wt % to at most 70 wt % of extragranular phase by the total weight of the pharmaceutical formulation.

In some embodiments the oral dosage form is formulated in a pharmaceutical formulation comprising at least 15 wt %; at least 20 wt %; preferably at least 25 wt %; preferably at least 28 wt %; preferably at least 30 wt %; preferably at least 34 wt %; preferably at least 35 wt % of intragranular phase by the total weight of the pharmaceutical formulation. In some embodiments the pharmaceutical formulation comprises at most 99 wt %; at most 93 wt %; preferably at most 85 wt %; preferably at most 80 wt %; preferably at most 75 wt %; preferably at most 74 wt %; preferably at most 73 wt %; preferably at most 70 wt %; preferably at most 67 wt %; preferably at most 65 wt %; preferably at most 63 wt %; preferably at most 60 wt %; preferably at most 55 wt %; preferably at most 53 wt % of intragranular phase by the total weight of the pharmaceutical formulation. In some embodiments the pharmaceutical formulation comprises at least 15 wt % to at most 75 wt %; preferably at least 25 wt % to at most 70 wt %; preferably at least 30 wt % to at most 65 wt %; preferably at least 35 wt % to at most 60 wt %; preferably at least 35 wt % to at most 70 wt % of intragranular phase by the total weight of the pharmaceutical formulation.

In some embodiments the oral dosage form is formulated in a pharmaceutical formulation comprising an intragranular phase comprising:
(i) from 5 wt % to 45 wt %, preferably from 10 wt % to 40 wt %, f preferably rom 15 wt % to 35 wt % of API by the total weight of the pharmaceutical formulation;
(ii) from 5 wt % to 60 wt %, preferably from 10 wt % to 50 wt %, preferably from 15 wt % to 55 wt % of methacrylic acid copolymer or hydroxypropyl methylcellulose or a combination thereof by the total weight of the pharmaceutical formulation;
(iii) from 5 wt % to 60 wt %, preferably from 10 wt % to 50 wt %, preferably from 15 wt % to 40 wt % of filler (e.g., mannitol, microcrystalline cellulose) by the total weight of the pharmaceutical formulation;
(iv) from 1 wt % to 10 wt %, preferably from 1.5 wt % to 9 wt %, preferably from 1.5 wt % to 8 wt % of disintegrant (e.g., croscarmellose sodium) by the total weight of the pharmaceutical formulation;
(v) from 0.1 wt % to 5 wt %, preferably from 0.2 wt % to 4.5 wt %, preferably from 0.5 wt % to 4 wt % of glidant (e.g., colloidal fumed silica) by the total weight of the pharmaceutical formulation;
(vi) from 0.1 wt % to 5 wt %, preferably from 0.2 wt % to 4.5 wt %, preferably from 0.5 wt % to 4 wt % of surfactant (e.g., sodium lauryl sulfate) by the total weight of the pharmaceutical formulation; and
(vii) from 0.1 wt % to 3 wt %, preferably from 0.2 wt % to 2.5 wt %, preferably from 0.5 wt % to 2 wt % of lubricant (e.g., magnesium stearate) by the total weight of the pharmaceutical formulation.

In some embodiments the oral dosage form is formulated in a pharmaceutical formulation comprising an extragranular phase comprising from 0.1 wt % to 5.0 wt %; preferably from 0.2 wt % to 4.7 wt %; preferably from 0.5 wt % to 4.5 wt %; preferably from 1 wt % to 3.5 wt %; preferably from 1.5 wt % to 3 wt % of disintegrant by the total weight of the pharmaceutical formulation.

In some embodiments the oral dosage form is formulated in a pharmaceutical formulation comprising an extragranular phase comprising from 0.1 wt % to 3 wt %; preferably from 0.2 wt % to 2.7 wt %; preferably from 0.5 wt % to 2.5 wt %; preferably from 0.8 wt % to 2 wt % of lubricant by the total weight of the pharmaceutical formulation.

In some embodiments the oral dosage form is formulated in a pharmaceutical formulation comprising an extragranular phase comprising from 0.1 wt % to 55 wt %; preferably from 0.2 wt % to 50 wt %; preferably from 0.3 wt % to 45 wt %; preferably from 0.4 wt % to 40 wt %; preferably from 0.5 wt % to 35 wt %; preferably from 0.6 wt % to 30 wt %; preferably from 0.7 wt % to 25 wt %; preferably from 0.8 wt % to 20 wt %; preferably 1 wt % to 15 wt %; preferably from 1.3 wt % to 12 wt %; preferably from 2 wt % to 10 wt %; preferably from 2.5 wt % to 9 wt %; preferably from 3 wt % to 8 wt %; preferably from 4 wt % to 7 wt %; preferably from 5 wt % to 6 wt % of filler by the total weight of the pharmaceutical formulation.

In some embodiments the oral dosage form is formulated in a pharmaceutical formulation comprising an extragranular phase comprising:
(a) from 0.1 wt % to 5.0 wt %, preferably from 0.2 wt % to 4.5 wt %, preferably from 0.5 wt % to 3 wt % of disintegrant (e.g., croscarmellose sodium) by the total weight of the pharmaceutical formulation;
(b) from 0.1 wt % to 3 wt %, preferably from 0.2 wt % to 2.5 wt %, preferably from 0.5 wt % to 2 wt % of lubricant (e.g. magnesium stearate) by the total weight of the pharmaceutical formulation; and
(c) from 1 wt % to 15 wt %, preferably from 1.5 wt % to 10 wt %, preferably from 2 wt % to 8 wt % of filler (e.g., hydroxypropyl methylcellulose) by the total weight of the pharmaceutical formulation.

In some embodiments, the oral dosage form of the invention is formulated in a pharmaceutical formulation comprising an intragranular phase and an extragranular phase; wherein the formulation comprises from 50 mg to 17000 mg of the intragranular phase, preferably from 80 mg to 10000 mg, preferably from 100 mg to 5000 mg, preferably from 120 mg to 3000 mg, preferably from 150 mg to 2500 mg, preferably from 200 mg to 2000 mg, preferably from 250 mg to 1200 mg of the intragranular phase, or any particular amount or range comprised therein.

In some embodiments, the oral dosage form of the invention is formulated in a pharmaceutical formulation comprising an intragranular phase and an extragranular phase; wherein the formulation comprises from 4 mg to 1500 mg of the extragranular phase, preferably from 6 mg to 1000 mg, preferably from 8 mg to 500 mg, preferably from 10 mg to 300 mg, preferably from 15 mg to 250 mg, preferably from 20 mg to 200 mg, preferably from 30 mg to 100 mg of the extragranular phase, or any particular amount or range comprised therein.

In some embodiments the oral dosage form is formulated in a pharmaceutical formulation comprising an intragranular phase comprising from 1 mg to 2000 mg; preferably from 5 mg to 1500 mg; preferably from 5 mg to 1000 mg; preferably from 10 mg to 500 mg; preferably from 15 mg to 400 mg; preferably from 20 mg to 350 mg of compound of formula (I), or any particular amount or range comprised therein.

In some embodiments the oral dosage form is formulated in a pharmaceutical formulation comprising an intragranular phase comprising from 20 mg to 6000 mg; preferably 30 mg to 4000 mg; preferably from 40 mg to 2000 mg; preferably from 70 mg to 1000 mg of one of methacrylic acid copolymer or hydroxypropyl methylcellulose or a combination thereof, or any particular amount or range comprised therein.

In some embodiments the oral dosage form is formulated in a pharmaceutical formulation comprising an intragranular phase comprising from 10 mg to 6500 mg, preferably from 12 mg to 5000 mg, preferably from 15 mg to 4000 mg, preferably from 15 mg to 2000 mg, preferably from 18 mg to 1000 mg, preferably from 18 mg to 500 mg of diluent/filler, or any particular amount or range comprised therein.

In some embodiments the oral dosage form is formulated in a pharmaceutical formulation comprising an intragranular phase comprising from 0.5 mg to 300 mg, preferably from 0.6 mg to 250 mg, preferably from 0.8 mg to 200 mg, preferably from 1 mg to 150 mg, preferably from 1.2 mg to 100 mg, preferably from 1.5 mg to 80 mg, preferably from 2 mg to 30 mg of surfactant, or any particular amount or range comprised therein.

In some embodiments the oral dosage form is formulated in a pharmaceutical formulation comprising an intragranular phase comprising from 2 mg to 720 mg, preferably from 4 mg to 650 mg, preferably from 5 mg to 400 mg, preferably from 6 mg to 300 mg, preferably from 7 mg to 200 mg, preferably from 7.5 mg to 100 mg, preferably from 8 mg to 60 mg of disintegrant, or any particular amount or range comprised therein.

In some embodiments the oral dosage form is formulated in a pharmaceutical formulation comprising an intragranular phase comprising from 1 mg to 400 mg, preferably from 2 mg to 350 mg, preferably from 3 mg to 300 mg, preferably from 4 mg to 200 mg, preferably from 5 mg to 100 mg, preferably from 6 mg to 50 mg, preferably from 8.5 mg to 35 mg of glidant, or any particular amount or range comprised therein.

In some embodiments the oral dosage form is formulated in a pharmaceutical formulation comprising an intragranular phase comprising from 0.3 mg to 90 mg; preferably from 0.5 mg to 70 mg; preferably from 0.6 mg to 50 mg; preferably from 0.7 mg to 25 mg; preferably from 0.8 mg to 10 mg of lubricant, or any particular amount or range comprised therein.

In some embodiments the oral dosage form is formulated in a pharmaceutical formulation comprising an intragranular phase comprising:
(i) from 1 mg to 2000 mg; preferably from 5 mg to 1500 mg; preferably; from 5 mg to 1000 mg; preferably from 10 mg to 500 mg; preferably from 15 mg to 400 mg; preferably from 20 mg to 350 mg of API;
(ii) from 20 mg to 6000; preferably 30 mg to 4000 mg; preferably from 40 mg to 2000 mg; preferably from 70 mg to 1000 mg of one of methacrylic acid copolymer or hydroxypropyl methylcellulose or a combination thereof;
(iii) from 10 mg to 6500 mg, preferably from 12 mg to 5000 mg, preferably from 15 mg to 4000 mg, preferably from 15 mg to 2000 mg, preferably from 18 mg to 1000 mg, preferably from 18 mg to 500 mg of diluent/filler (e.g., mannitol, microcrystalline cellulose);
(iv) from 2 mg to 720 mg, preferably from 4 mg to 650 mg, preferably from 5 mg to 400 mg, preferably from 6 mg to 300 mg, preferably from 7 mg to 200 mg, preferably from 7.5 mg to 100 mg, preferably from 8 mg to 60 mg of disintegrant (e.g., croscarmellose sodium);
(v) from 1 mg to 400 mg, preferably from 2 mg to 350 mg, preferably from 3 mg to 300 mg, preferably from 4 mg to 200 mg, preferably from 5 mg to 100 mg, preferably from 6 mg to 50 mg, preferably from 8.5 mg to 35 mg of glidant (e.g., colloidal fumed silica);
(vi) from 0.5 mg to 300 mg, preferably from 0.6 mg to 250 mg, preferably from 0.8 mg to 200 mg, preferably from 1 mg to 150 mg, preferably from 1.2 mg to 100 mg, preferably from 1.5 mg to 80 mg, preferably from 2 mg to 30 mg of surfactant (e.g., sodium lauryl sulfate); and
(vii) from 0.3 mg to 90 mg; preferably from 0.5 mg to 70 mg; preferably from 0.6 mg to 50 mg; preferably from 0.7 mg to 25 mg; preferably from 0.8 mg to 10 mg of lubricant (e.g., magnesium stearate).

In some embodiments the oral dosage form is formulated in a pharmaceutical formulation comprising an extragranular phase comprising from 0.6 mg to 180 mg; preferably from 1 mg to 100 mg; preferably from 2 mg to 80 mg; preferably from 3 mg to 50 mg; preferably from 5 mg to 20 mg of disintegrant.

In some embodiments the oral dosage form is formulated in a pharmaceutical formulation comprising an extragranular phase comprising from 3.5 mg to 1100 mg; preferably from 5 mg to 500 mg; preferably from 10 mg to 250 mg; preferably from 15 mg to 100 mg; preferably from 25 mg to 90 mg of diluent/filler.

In some embodiments the oral dosage form is formulated in a pharmaceutical formulation comprising an extragranular phase comprising from 0.3 mg to 90 mg; preferably from 0.5 mg to 70 mg; preferably from 1 mg to 50 mg; preferably from 1.5 mg to 20 mg; preferably from 2 mg to 10 mg of lubricant.

In some embodiments the oral dosage form is formulated in a pharmaceutical formulation comprising an extragranular phase comprising:
(a) from 0.6 mg to 180 mg; preferably from 1 mg to 100 mg; preferably from 2 mg to 80 mg; preferably from 3 mg to 50 mg; preferably from 5 mg to 20 mg of disintegrant (e.g., croscarmellose sodium);
(b) from 0.3 mg to 90 mg; preferably from 0.5 mg to 70 mg; preferably from 1 mg to 50 mg; preferably from 1.5 mg to 20 mg; preferably from 2 mg to 10 mg of lubricant (e.g. magnesium stearate); and
(c) from 3.5 mg to 1100 mg; preferably from 5 mg to 500 mg; preferably from 10 mg to 250 mg; preferably from 15 mg to 100 mg; preferably from 25 mg to 90 mg of diluent/filler (e.g., hydroxypropyl methylcellulose).

One skilled in the art will readily recognize that the appropriate pharmaceutically acceptable excipients are selected such that they are compatible with other excipients and do not bind with the active pharmaceutical ingredient (i.e., compound of formula (I)) or cause degradation.

It will be appreciated that any of the above description relating to components of the pharmaceutical formulation may apply to any of the other aspects and embodiments of the invention.

The present invention provides a compound of formula (I) as described herein, for use in the prevention and/or treatment of dengue viral infections, wherein the compound is comprised in a pharmaceutical formulation which is administered in either the fed or fasted state. Preferably the compound is administered to a human at risk of being infected by Dengue virus or is already infected by Dengue virus.

In some embodiments, the compound of formula (I) is in amorphous form or dissolved state (i.e., molecular dispersion) in the pharmaceutical formulation.

In additional embodiments of the invention the compound of formula (I) is selected from the group consisting of:

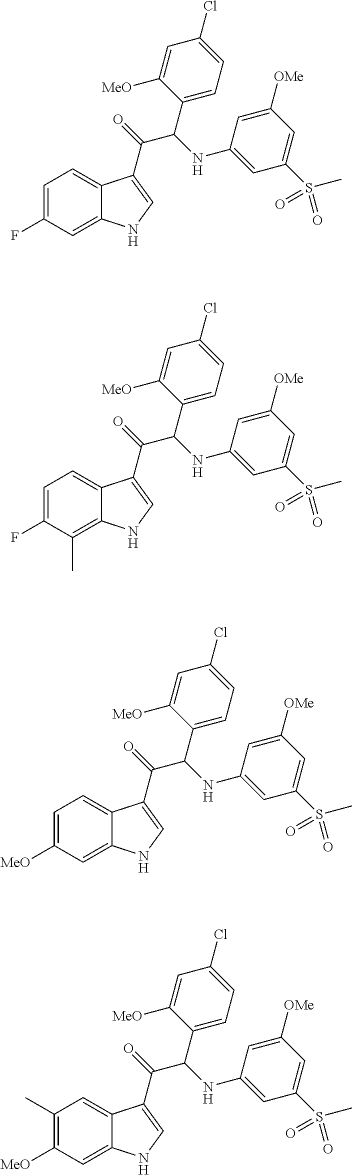 [SMILES: COc1cc(NC(C(=O)c2c[nH]c3cc(F)ccc23)c2ccc(Cl)cc2OC)cc(S(C)(=O)=O)c1 ; COc1cc(NC(C(=O)c2c[nH]c3c(C)c(F)ccc23)c2ccc(Cl)cc2OC)cc(S(C)(=O)=O)c1 ; COc1cc(NC(C(=O)c2c[nH]c3cc(OC)ccc23)c2ccc(Cl)cc2OC)cc(S(C)(=O)=O)c1 ; COc1cc(NC(C(=O)c2c[nH]c3cc(OC)c(C)cc23)c2ccc(Cl)cc2OC)cc(S(C)(=O)=O)c1]

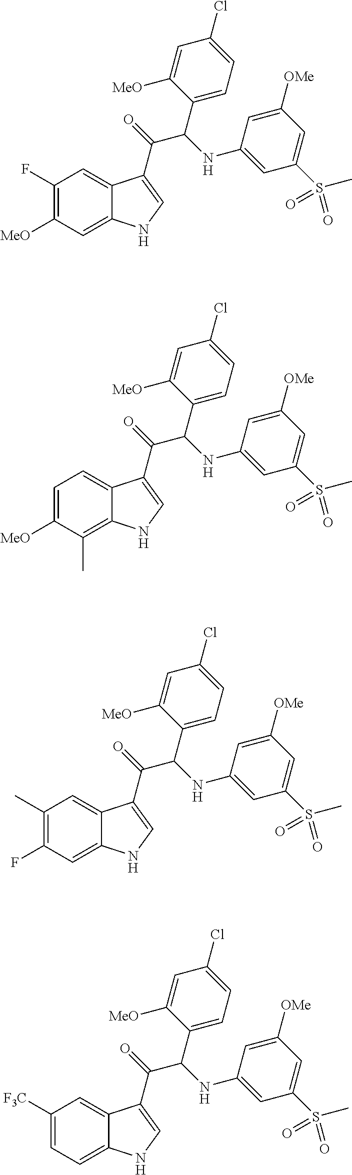 [SMILES: COc1cc(NC(C(=O)c2c[nH]c3cc(OC)c(F)cc23)c2ccc(Cl)cc2OC)cc(S(C)(=O)=O)c1 ; COc1cc(NC(C(=O)c2c[nH]c3c(C)c(OC)ccc23)c2ccc(Cl)cc2OC)cc(S(C)(=O)=O)c1 ; COc1cc(NC(C(=O)c2c[nH]c3cc(F)c(C)cc23)c2ccc(Cl)cc2OC)cc(S(C)(=O)=O)c1 ; COc1cc(NC(C(=O)c2c[nH]c3ccc(C(F)(F)F)cc23)c2ccc(Cl)cc2OC)cc(S(C)(=O)=O)c1]

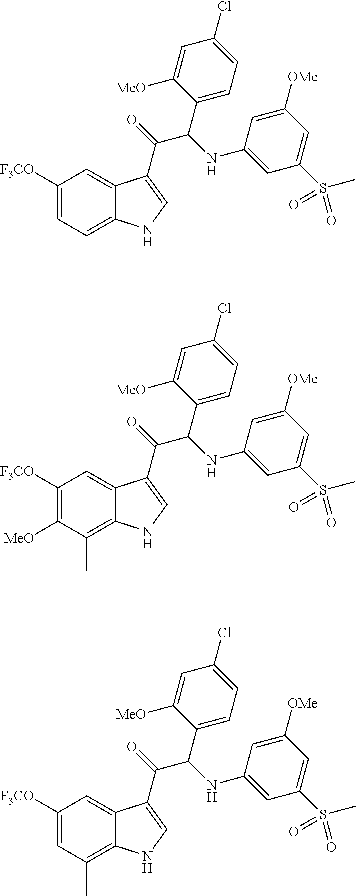 [SMILES: COc1cc(NC(C(=O)c2c[nH]c3ccc(OC(F)(F)F)cc23)c2ccc(Cl)cc2OC)cc(S(C)(=O)=O)c1 ; COc1cc(NC(C(=O)c2c[nH]c3c(C)c(OC)c(OC(F)(F)F)cc23)c2ccc(Cl)cc2OC)cc(S(C)(=O)=O)c1 ; COc1cc(NC(C(=O)c2c[nH]c3c(C)cc(OC(F)(F)F)cc23)c2ccc(Cl)cc2OC)cc(S(C)(=O)=O)c1]

or its stereo-isomeric form, a pharmaceutically acceptable salt, solvate or polymorph thereof.

The present invention also comprises the compound of formula (I), or an enantiomer, diastereomer or pharmaceutically acceptable salt form thereof.

The present invention also comprises the compound of formula (I), or an enantiomer, diastereomer or pharmaceutically acceptable salt form thereof, in amorphous state or dissolved state (i.e., molecular dispersion).

In particular, the starting material in the process to prepare a pharmaceutical formulation as described herein, is a compound of formula (I), or an enantiomer, diastereomer, solvate, or a pharmaceutically acceptable salt form thereof; while the final pharmaceutical formulation or solid dosage form as defined herein also comprises a compound of formula (I), or an enantiomer, diastereomer, or pharmaceutically acceptable salt form thereof, in amorphous form or dissolved state.

In a preferred embodiment, the compound of formula (I) is

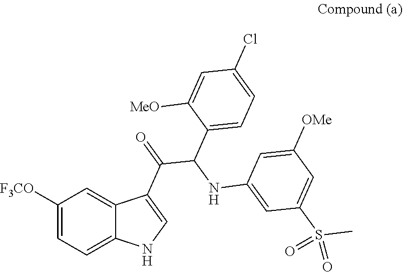 [SMILES: COc1cc(NC(C(=O)c2c[nH]c3ccc(OC(F)(F)F)cc23)c2ccc(Cl)cc2OC)cc(S(C)(=O)=O)c1]

or a stereo-isomeric form, a pharmaceutically acceptable salt, solvate or polymorph thereof.

The compound of formula (I) may be Compound (a) or a solvate or pharmaceutically acceptable salt form thereof. The compound of formula (I) may be Compound (a) or a pharmaceutically acceptable salt form thereof. The compound of formula (I) may be Compound (a) in a solvated form, for example as a monohydrate. The compound of formula (I) may be Compound (a) in a solvated with an organic solvent and water, e.g., a solvate and a hydrate. Preferably the compound of formula (I) is Compound (a). Preferably the compound of formula (I) is the (+)-enantiomer of Compound (a). Preferably the compound of formula (I) is the(S)-enantiomer of Compound (a). Preferably the compound of formula (I) is Compound (a) in anhydrous form. Preferably the compound of formula (I) is Compound (a) in amorphous form. Preferably the compound of formula (I) is Compound (a) or a pharmaceutically acceptable salt form thereof in amorphous form or dissolved state. Preferably the compound of formula (I) is Compound (a) in amorphous form or dissolved state. Preferably the compound of formula (I) is the(S)-enantiomer of Compound (a) in amorphous form. Preferably the compound of formula (I) is the(S)-enantiomer of Compound (a) in anhydrous form.

In some embodiments, the compound of formula (I) is: Enantiomer 9A,

wherein

¹H NMR (400 MHZ, DMSO-d₆) δ ppm 3.09 (s, 3H) 3.73 (s, 3H) 3.99 (s, 3H) 6.26 (d, J=7.9 Hz, 1H) 6.55-6.62 (m, 2H) 6.91 (t, J=1.5 Hz, 1H) 6.98 (dd, J=8.4, 2.0 Hz, 1H) 7.07 (d, J=7.9 Hz, 1H) 7.13 (d, J=2.0 Hz, 1H) 7.21 (dd, J=8.8, 1.8 Hz, 1H) 7.36 (d, J=8.4 Hz, 1H) 7.59 (d, J=8.8 Hz, 1H) 8.07 (d, J=0.9 Hz, 1H) 8.55 (s, 1H) 12.29 (br s, 1H).

In some embodiments, the compound of formula (I) is: Enantiomer 9B,

wherein

¹H NMR (400 MHZ, DMSO-d₆) δ ppm 3.09 (s, 3H) 3.73 (s, 3H) 3.99 (s, 3H) 6.26 (d, J=7.9 Hz, 1H) 6.56-6.62 (m, 2H) 6.92 (t, J=2.0 Hz, 1H) 6.98 (dd, J=8.1, 2.0 Hz, 1H) 7.07 (d, J=7.9 Hz, 1H) 7.13 (d, J=2.0 Hz, 1H) 7.22 (dd, J=8.8, 1.8 Hz, 1H) 7.36 (d, J=8.4 Hz, 1H) 7.59 (d, J=8.8 Hz, 1H) 8.07 (d, J=0.9 Hz, 1H) 8.55 (s, 1H) 12.30 (br s, 1H).

Compounds of formula (I) can be synthesized according to the procedures disclosed in WO 2016/180696, which is incorporated herein by reference in its entirety.

Compounds of formula (I) for use in the present invention can be synthesized in accordance with the general synthetic methods described below and illustrated in the schemes and examples that follow. Since the schemes are an illustration, the invention should not be construed as being limited by the chemical reactions and conditions described in the schemes and examples. Compounds analogous to the target compounds of these examples can be made according to similar routes. The disclosed compounds are useful as pharmaceutical agents as described herein. The various starting materials used in the schemes and examples are commercially available or may be prepared by methods well within the skill of persons versed in the art.

The synthesis of compounds of general formula (I) can be performed as outlined in Scheme 1. 2-(4-Chloro-2-methoxyphenyl) acetic acid (II) can be converted to the corresponding 2-(4-chloro-2-methoxyphenyl) acetyl chloride (III) with a chlorination reagent like for example thionyl chloride. The Friedel-Crafts reaction of the acid chloride III with a substituted indole of general formula IV can be performed using a Lewis acid reagent like for example Et₂AlCl or TiCl₄ in a suitable solvent like for example CH₂Cl₂ or 1,2-dichloroethane, and under suitable reaction conditions that typically (but not exclusively) involve cooling, to provide the 3-acylated indole of general formula V. The introduction of an aniline moiety in alpha position to the carbonyl moiety of the compounds of general formula V can be accomplished by a reaction sequence that involves for example bromination of V with a reagent like for example phenyltrimethylammonium tribromide in a suitable solvent like for example THF (tetrahydrofuran), to provide the compounds of general formula VI, and subsequent reaction of the compounds of general formula VI with 3-methoxy-5-(methylsulfonyl) aniline (VII) in a suitable solvent like for example CH₃CN, and typically using a base like for example TEA or DIPEA, to provide the compounds of general formula I as racemic mixtures. Chiral separation of the compounds of general formula I can be performed by for example chiral chromatography to provide the Enantiomers A and B of general formula (I).

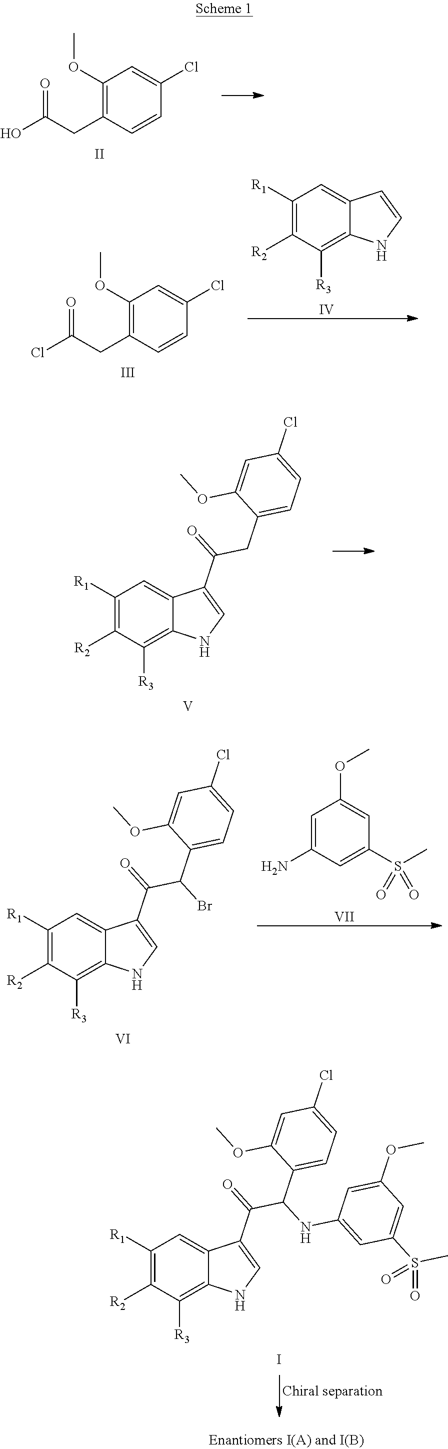

In some cases, the synthesis of the intermediate of general formula V via the Friedel-Crafts synthesis approach, benefits from the presence of a protecting group (PG) at the indole-N during the Friedel-Crafts reaction step, as outlined in Scheme 2. To this end, the substituted indole of general formula IV can be converted first to an N-protected intermediate of general formula VIII, such as for example an N-Tosylated intermediate of general formula VIII (PG=Ts), using a reagent like for example tosyl chloride, in the presence of a base like for example sodium hydride. The Friedel-Crafts reaction of the substituted indole of general formula IV with acid chloride III can be performed using a Lewis acid reagent like for example Et₂AlCl or TiCl₄ in a suitable solvent like for example CH₂Cl₂ or 1,2-dichloroethane, and under suitable reaction conditions that typically (but not exclusively) involve cooling, to provide the 3-acylated N-protected indole of general formula IX. Removal of the indole-N protecting group PG of the intermediate of general formula IX can be accomplished with a reagent like for example LiOH (for PG=Ts) in a solvent mixture like for example THF/water an at a suitable reaction temperature, to provide the 3-acylated indole of general formula V.

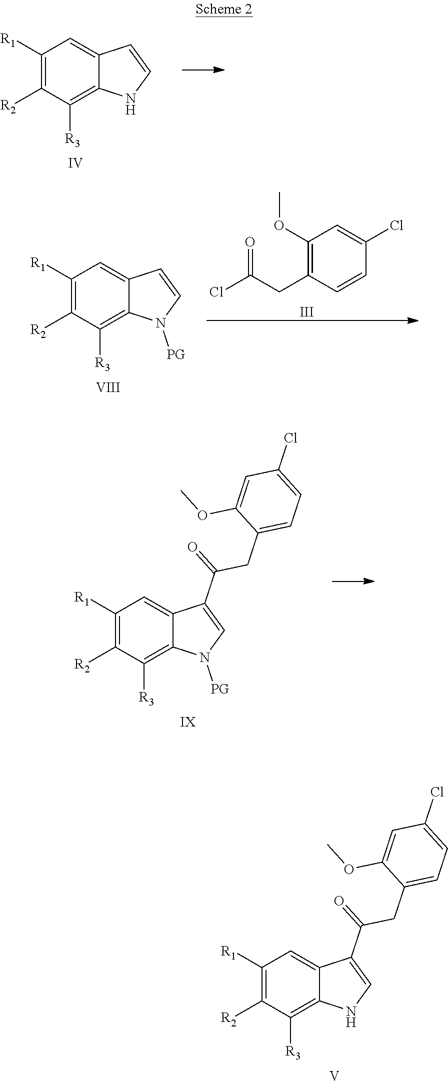

As an alternative approach, the intermediate of general formula V can also be prepared as outlined in Scheme 3: The N-Boc-protected substituted indole-3-carbaldehyde of general formula X can be converted to the corresponding Strecker-type of intermediate of general formula XI by reaction with morpholine in the presence of reagents like for example sodium cyanide and sodium bisulfite and in a suitable solvent like for example a mixture of water and a water-mixable organic solvent like for example dioxane. Alkylation of the compound of general formula XI with 4-chloro-2-methoxy-benzylchloride can be accomplished in the presence of a base like for example potassium hexamethyldisilazane and in a suitable solvent like for example DMF to provide the compound of general formula XII. Submission of the compound of general formula XII to a suitable aqueous acidic hydrolytic condition like for example by treatment with an aqueous hydrochloric acid solution at elevated temperature, provides the intermediate of general formula V.

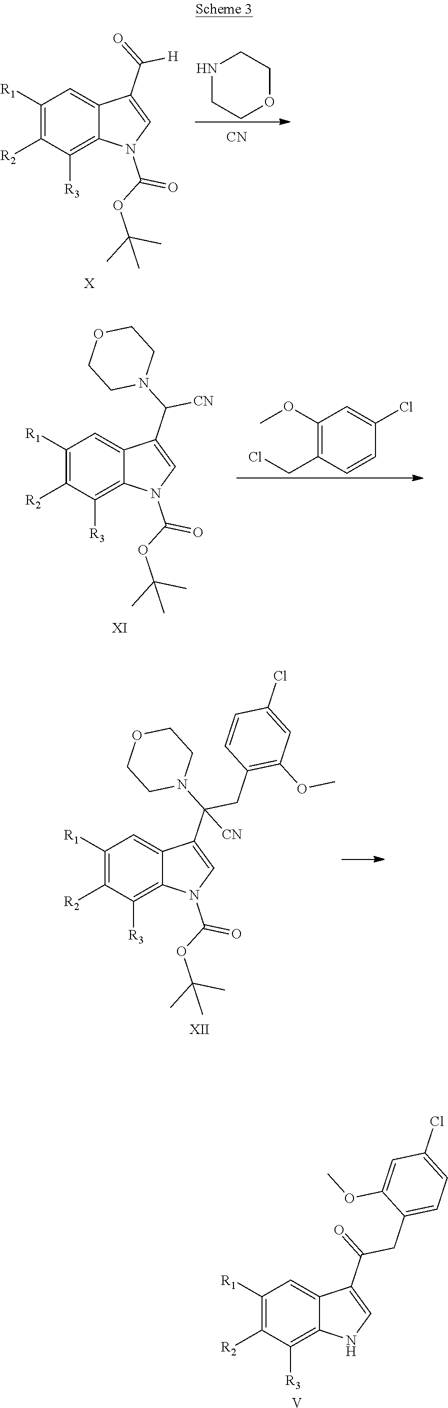

In a particular embodiment, the compound of formula (I) is Compound (a), or a stereo-isomeric form, a pharmaceutically acceptable salt, solvate or polymorph thereof.

The invention also provides a compound of formula (I) as described herein, wherein the compound is comprised in a pharmaceutical formulation which is administered as solid dosage form as described herein.

The dosage form may be an oral dosage form (e.g., a capsule for oral administration). Alternatively, the dosage form may be an enteral dosage form. The solid dosage form may alternatively be a tablet.

The solid dosage form as described herein (e.g., a tablet) may contain from 0.1 mg to 3000 mg of the compound of formula (I), preferably from 1 mg to 2000 mg of compound of formula (I), preferably from 5 mg to 1500 mg of compound of formula (I), preferably from 5 mg to 1000 mg of compound of formula (I), preferably from 10 mg to 1100 mg of compound of formula (I), preferably from 15 mg to 950 mg of the compound of formula (I), preferably from 20 mg to 900 mg of compound of formula (I) or any particular amount or range comprised therein.

The solid dosage form as described herein (e.g., a tablet) may contain from 0.5 mg to 2000 mg of the compound of formula (I) or any particular amount or range comprised therein.

In some embodiments the solid dosage form may comprise from 0.5 mg to 1800 mg, preferably from 1 mg to 1600 mg, preferably from 2 mg to 1500 mg, preferably from 3 mg to 1450 mg, preferably from 3 mg to 1300 mg, preferably from 4 mg to 1200 mg, preferably from 5 mg to 1100 mg, preferably from 6 mg to 1000 mg, preferably from 6 mg to 900 mg, preferably from 7 mg to 800 mg, preferably from 8 mg to 700 mg, preferably from 9 mg to 600 mg, preferably from 10 mg to 500 mg, preferably from 11 mg to 400 mg, preferably from 12 mg to 300 mg, preferably from 13 mg to 200 mg, preferably from 14 mg to 100 mg, preferably from 15 mg to 50 mg, or any particular amount or range comprised therein; preferably the compound of formula (I) is Compound (a) shown below

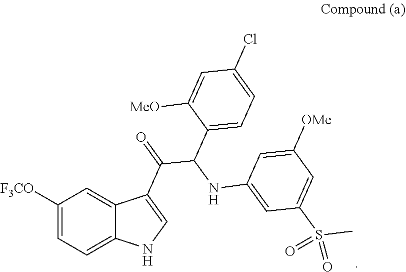 [SMILES: COc1cc(NC(C(=O)c2c[nH]c3ccc(OC(F)(F)F)cc23)c2ccc(Cl)cc2OC)cc(S(C)(=O)=O)c1]

Preferably the compound of formula (I) is the (+)-enantiomer of Compound (a), preferably the(S)-enantiomer of Compound (a).

The solid dosage form may comprise at least 2 mg of Compound (a), preferably at least 10 mg, preferably at least 15 mg, preferably at least 20 mg, preferably at least 30 mg, preferably at least 40 mg of Compound (a).

In a particular embodiment, the solid dosage form is a tablet comprising
a) a compound of formula (I); and
b1) methacrylic acid copolymer, or
b2) a cellulose derivative such as hydroxypropyl methylcellulose (HPMC).

In a particular embodiment, the solid dosage form is a tablet comprising
a) a compound of formula (I); and
b1) methacrylic acid copolymer, or
b2) a cellulose derivative such as hydroxypropyl methylcellulose (HPMC).
c) one or more pharmaceutically acceptable excipients selected from the group comprising disintegrants, binders, diluents, lubricants, stabilizers, wetting agents, glidants, osmotic agents, colorants, plasticizers, coatings, fillers and surfactants.

In a particular embodiment, the solid dosage form is a tablet comprising a pharmaceutical formulation of the present invention.

In an embodiment, the solid dosage form comprises a pharmaceutical formulation, wherein the formulation comprises at least 5 mg of a compound of formula (I), preferably at least 10 mg, preferably at least 15 mg, preferably at least 20 mg, preferably at least 25 mg, preferably at least 30 mg, preferably at least 35 mg, preferably at least 40 mg, of compound of formula (I); preferably the compound of formula (I) is

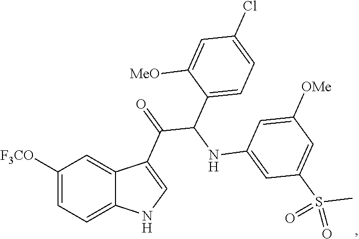 [SMILES: COc1cc(NC(C(=O)c2c[nH]c3ccc(OC(F)(F)F)cc23)c2ccc(Cl)cc2OC)cc(S(C)(=O)=O)c1]

or a pharmaceutically acceptable salt form thereof.

For oral administration, a solid dosage form is in particular provided in the form of tablets containing at least 1 mg of compound of formula (I), preferably at least 10 mg, preferably at least 15 mg, preferably at least 20 mg, preferably at least 25 mg, preferably at least 30 mg, preferably at least 40 mg; in particular from 15 mg to 2500 mg of compound of formula (I).

It will be appreciated that any of the above description relating to solid dosage forms may apply to any of the other aspects and embodiments of the invention.

Advantageously, the compound of formula (I) may be administered in a single daily dose, or the total daily dosage may be administered in divided doses of two, three, four or five daily.

In some embodiments the compound of formula (I) is administered in either a fed or fasted state.

It will be appreciated that any of the above description relating to oral dosage forms may apply to any of the other aspects and embodiments of the invention.

The invention also provides a process for preparing a pharmaceutical formulation as described herein, the process comprising the steps of:
a) dissolving the compound of formula (I) in a solvent to form a solution;
b) mixing methacrylic acid copolymer or hydroxypropyl methylcellulose or a combination thereof with the solution formed in step a) thereby obtaining a mixture;
c) spray drying the mixture to obtain a solid dispersion;
d) optionally blending the solid dispersion with at least one pharmaceutically acceptable excipient;
to provide a pharmaceutical formulation as described herein.

As used herein the term “solid dispersion” means the dispersion of an API (i.e., a compound of formula (I)) in a solid matrix where the matrix comprises a small molecule or a polymer or a combination thereof.

The pharmaceutical formulation according to the present invention comprises a solid dispersion wherein the matrix is a polymer and the polymer is selected from methacrylic acid copolymer, or a cellulose derivative such as methyl cellulose (MC), ethyl cellulose (EC), hydroxyethyl cellulose (HEC), hydroxypropyl cellulose (HPC), carboxymethyl cellulose (CMC), sodium carboxymethyl cellulose (NaCMC) or hydroxypropyl methylcellulose (HPMC), or a combination thereof. Preferably, the cellulose derivative is HPMC.

The invention also provides a process for preparing a solid dosage form described herein, the process comprising the steps of:
a) dissolving the compound of formula (I) in a solvent to form a solution;
b) mixing methacrylic acid copolymer or a cellulose derivative such as methyl cellulose (MC), ethyl cellulose (EC), hydroxyethyl cellulose (HEC), hydroxypropyl cellulose (HPC), carboxymethyl cellulose (CMC), sodium carboxymethyl cellulose (NaCMC) or hydroxypropyl methylcellulose, or a combination thereof with the solution formed in step a) thereby obtaining a mixture, and optionally further stirring the mixture;
c) spray drying the mixture to obtain a solid dispersion;
d) optionally blending the solid dispersion with one or more pharmaceutically acceptable excipients; for example, the pharmaceutically acceptable excipient can be selected from the group comprising fillers, surfactants, disintegrants, glidants, lubricants and mixture thereof;
e) compressing the blend into a tablet;
to provide a solid dosage form as described herein.

In some embodiments, the process for preparing a solid dosage form described herein, comprises the steps of:
a) dissolving the compound of formula (I) in a solvent to form a solution;
b) mixing the methacrylic acid copolymer or hydroxypropyl methylcellulose or a combination thereof with the solution formed in step a) thereby obtaining a mixture, and optionally further stirring the mixture;
c) spray drying the mixture to obtain a solid dispersion;
d) blending the solid dispersion with at least one filler, at least one surfactant, at least one disintegrant, at least one glidant and at least one lubricant;
e) granulating the blend;
f) blending the mixture obtained in step e) with at least one disintegrant, filler and at least one lubricant;
g) compressing the blend into a tablet;
to provide a solid dosage form as described herein.

In some embodiments, the solid dispersion may be obtained using hot melt extrusion. In some embodiments the step of granulating the blend is performed using a roller compactor or by slugging.

Another aspect of the present invention provides a packaged pharmaceutical formulation for use in the prevention and/or treatment of dengue viral infections, wherein the pharmaceutical formulation is administered in an oral dosage form to a human subject in either a fed or fasted state, preferably in a fed state, and wherein the pharmaceutical formulation is any formulation described herein (e.g., a tablet) sealed in a blister film, wherein the blister film comprises a formulation retaining layer configured to hold one or more pharmaceutical formulation (e.g., tablets) and a sealing layer configured to overlay the retaining layer to seal the pharmaceutical formulation(s) within the retaining layer, wherein the sealing layer comprises aluminum foil and a desiccant material. As used herein, the term “desiccant material” refers to any hygroscopic substance useful as a drying agent. Examples of desiccant materials include without limitation silica (e.g., silica gel), activated charcoal, calcium sulfate, calcium chloride, and zeolite materials.

In some embodiments, the retaining layer comprises one or more chambers, wherein each chamber is configured to hold one or more pharmaceutical formulations (such as any pharmaceutical formulation described herein (e.g., one or more tablets), and each chamber is sealed by the sealing layer. In some embodiments, the retaining layer comprises a clear or opaque material (e.g., a clear or opaque polyethylene material). In some embodiments, the sealing layer entirely overlaps the retaining layer and any chambers provided in the retaining layer.

Examples of commercially available blister films useful for the present invention include Dessiflex Plus and Dessiflex Ultra available from Amcor plc. In some embodiments, the packaged pharmaceutical formulation consists of 1 or more (e.g., 1, 2, 3, 4, 5, 6, 7, 8, 9, 10, 1 to 4, 2 to 10, or 1 to 10) tablets sealed in the blister film, wherein the blister film comprises one card. The stability and shelf life of the pharmaceutical formulations of the instant invention are improved using the blister film packaging of the instant invention, wherein the sealing layer comprises a desiccant material, as compared to packaging the pharmaceutical formulation in a blister film having a sealing layer that lacks a desiccant material (e.g., Aclar 400 blister film).

Another aspect of the present invention provides a kit for use in the prevention and/or treatment of dengue viral infections, wherein the kit is administered in an oral dosage form to a human subject in either a fed or fasted state, preferably in a fed state, and comprising a packaged pharmaceutical formulation, such as any packaged pharmaceutical formulation described herein, and instructions for the administration of the packaged pharmaceutical formulation.

It will be appreciated that any of the above discussion relating to solid dosage form, formulation, and processes for their preparation may apply to any embodiments of method of prevention and/or treatments described herein.

The present invention further encompasses a method for treating, ameliorating and/or preventing a disease, a syndrome, a condition that is affected by the inhibition of dengue viral replication, most preferably in a human subject.

The method comprises administering to the subject a therapeutically effective amount of the compound of formula (I), such as Compound (a), or pharmaceutical formulations and/or solid dosages comprising said compound of formula (I) as described herein in accordance with any of the dosing regimens described herein. The subject is at risk of being infected by Dengue virus or infected by Dengue virus.

One embodiment of the present invention is directed to a method of preventing a dengue viral infection in a human subject in need thereof, comprising administering to the subject a therapeutically effective amount of a pharmaceutical formulation and/or solid dosages comprising compound of formula (I), such as Compound (a), as described herein in accordance with any of the dosing regimens described herein, wherein the subject is in a fed state.

One embodiment of the present invention is directed to a method of treating a dengue viral infection in a human subject in need thereof, comprising administering to the subject a therapeutically effective amount of a pharmaceutical formulation and/or solid dosages comprising compound of formula (I), such as Compound (a), as described herein in accordance with any of the dosing regimens described herein, wherein the subject is in a fed state.

The present invention further encompasses a method of producing blood plasma levels of a compound of formula (I), in particular of Compound (a), as described herein, that are sufficient in the treatment or prevention of Dengue viral infection, preferably in a human subject. The method comprises administering to the subject, preferably a human subject, a therapeutically effective amount of the compound of formula (I), in particular of Compound (a), or pharmaceutical formulations and/or solid dosages comprising said compound of formula (I), in particular Compound (a), as described herein in accordance with any of the dosing regimens described herein. The subject is at risk of being infected by Dengue virus or infected by Dengue virus.

Preferably the amount of compound of formula (I), in particular Compound (a), that is administered to the subject, preferably a human subject, in the methods and uses described herein, is safe or effective in producing a blood plasma level of compound of formula (I) that is sufficient to treat or prevent dengue during the dosing time interval in said subject.

In another embodiment of the present invention, the pharmaceutical formulations or oral dosage form described herein may be employed in combination with one or more other medicinal agents, more particularly with other antiviral agents.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral formulations can also include adjuvants such as wetting agents, emulsifying and suspending agents, sweetening, flavoring, and perfuming agents.

Solid dosage forms for oral administration include capsules, tablets, pills, powders, and granules, preferably tablets. In such solid dosage forms, the active compound is mixed with at least one inert, pharmaceutically acceptable excipient or carrier such as sodium citrate or dicalcium phosphate and/or a) diluents or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid formulations of a similar type may also be employed as diluents in soft and hard-filled gelatin capsules using such pharmaceutically acceptable excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a formulation that they release the active ingredient(s) only, or preferentially, in a certain part of the intestinal tract, optionally, in a delayed manner. Examples of embedding formulations that can be used include polymeric substances and waxes. Solid formulations of a similar type may also be employed as diluents in soft and hard-filled gelatin capsules using such pharmaceutically acceptable excipients as lactose or milk sugar as well as high molecular weight polyethylene glycols and the like.

The active compounds can also be in microencapsulated form with one or more pharmaceutically acceptable excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a formulation that they release the active ingredient(s) only, or preferentially, in a certain part of the intestinal tract, optionally, in a delayed manner. Examples of embedding formulations that can be used include polymeric substances and waxes.

The pharmaceutical formulations described herein may be orally administered in any orally acceptable dosage form including, but not limited to, capsules, tablets, aqueous suspensions or solutions. In the case of tablets for oral use, carriers commonly used include, but are not limited to, lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

The compounds for use in the methods of the invention can be formulated in unit dosage form. The term “unit dosage form” refers to physically discrete units suitable as unitary dosage for subjects undergoing prevention and/or treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., 1 to 4 or more times per day). When multiple daily doses are used, the unit dosage form can be the same or different for each dose.

All possible combinations of the above-indicated embodiments are considered to be embraced within the scope of this invention.

Reference is now made to the following examples, which illustrate the invention in a non-limiting fashion.

### EXAMPLES

In the following examples Compound (a), preferably the (+)-enantiomer of Compound (a) was used as active pharmaceutical ingredient (API). Compound (a) was synthesized as described in WO 2016/180696, under Example 9. It was obtained as a white powder:

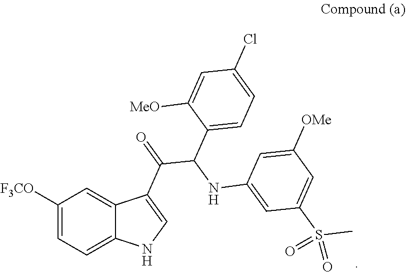 [SMILES: COc1cc(NC(C(=O)c2c[nH]c3ccc(OC(F)(F)F)cc23)c2ccc(Cl)cc2OC)cc(S(C)(=O)=O)c1]

### Reagents

| Chemical Name | | Company |
|---|---|---|
| Methacrylic acid -methyl | Eudragit L100 | Evonik |
| methacrylate copolymer | | |
| hydroxypropyl methylcellulose | HPMC E5 | Dow |
| E5 | | |
| Hydroxypropyl methylcellulose | HPMC-AS | Shin-Etsu |
| acetate succinate | | |
| Hydroxypropyl methylcellulose | HPMC AS MG | Shin-Etsu |
| acetate succinate | | |
| Polyvinylpyrrolidone | PVP K30 | BASF |
| Croscarmellose sodium: | Ac-Di-Sol ® | DuPont Pharma |
| Microcrystalline cellulose: | Avicel ® PH102 | DuPont Pharma |
| Colloidal fumed silica | Aerosil ® 200 | Evonik |
| Magnesium Stearate | N/A | Avantor |
| Sodium lauryl sulfate | Kolliphor ® SLS Fine | BASF |
| Mannitol | N/A | Roquette |
| polyethylene glycol 400 | PEG400 | Croda |

### Example 1

The relative oral bioavailability of Compound (a) administered as 2 different solid oral dosage forms formulated in two different pharmaceutical formulations, compared to an oral solution was determined in healthy adult participants in a randomized single dose, 2-period crossover study. The participants were in fasted conditions.

For the oral solution (OS), Compound (a) was provided as neat powder, e.g., 4000 mg per bottle for oral solution. This powder was reconstituted with an appropriate volume of diluent, i.e. polyethylene glycol 400 [PEG400] to obtain a 160 mg/mL oral solution.
Solid formulation #1 (SF1) was a tablet formulation comprising either 50 mg (SF1a) or 200 mg of Compound (a) (SF1b) per tablet.
Solid formulation #2 (SF2) was a tablet formulation comprising either 50 mg (SF2a) or 200 mg of Compound (a) (SF2b) per tablet.
SF1a/b and SF2a/b tablets were prepared as described below:
1. The API (added as a solid dispersion (SD) powder), microcrystalline cellulose, mannitol, croscarmellose sodium, colloidal silicon dioxide, magnesium stearate, sodium lauryl sulfate, were sieved together and then blended in a bin blender.
2. The blend was roller compacted in a roller compactor WP-120 and the resulting granules (corresponding to the intragranular phase) were collected.
3. Croscarmellose sodium and microcrystalline cellulose were sieved together, and added to the dry granules obtained in step 2, the mixture was blended in a bin blender.
4. Magnesium stearate was added and the resulting mixture blended in a bin blender.
5. The final blend was compressed into tablets using a XL 100 Korsch tablet press.

Table 1 shows the composition of the intragranular (Intra RC) and the extragranular phase (Extra RC) for each of SF1a/b and SF2a/b.

TABLE 1
Composition of SF1a/b and SF2a/b

| | | SF1a | SF2a | SF1b | SF2b |
|---|---|---|---|---|---|
| | | 50 mg API | 50 mg API | 200 mg API | 200 mg API |
| | Ingredient | (mg/tablet) | (mg/tablet) | (mg/tablet) | (mg/tablet) |
| Intra RC | SD1a Powder | 150 | — | — | — |
| | SD2a Powder | — | 150 | — | — |
| | SD1b Powder | — | — | 600 | — |
| | SD2b Powder | — | — | — | 600 |
| | Mannitol | 24 | 24 | 96 | 96 |
| | Microcrystalline cellulose | 80 | 80 | 320 | 320 |
| | Sodium lauryl sulfate | 4.5 | 4.5 | 18 | 18 |
| | Croscarmellose Sodium | 12 | 12 | 48 | 48 |
| | Colloidal silicon dioxide | 6 | 6 | 24 | 24 |
| | Magnesium Stearate | 1.5 | 1.5 | 6 | 6 |
| Extra RC | Croscarmellose Sodium | 3 | 3 | 12 | 12 |
| | Microcrystalline cellulose | 17.5 | 17.5 | 70 | 70 |
| | Magnesium Stearate | 1.5 | 1.5 | 6 | 6 |

| | | |
|---|---|---|
| Total | 1200 | 1200 |

The SD1a/b/SD2a/b were prepared following the procedure shown below:
1. Methanol (for SD1 Powder) or a mixture of methanol and methylene chloride (1/1, v/v, for SD2 Powder) were transferred into a container and stirred using a mixer. While stirring, Compound (a) was added to the solvent. Stirring continued until Compound (a) dissolved (see Table 2 for amounts). The resulting solution was filtered.
2. Eudragit L100 (for SD1 Powder) or hydroxypropyl methylcellulose E5 (HPMC E5) (for SD2 Powder) were added to the solution from step 1. The mixture was stirred until fully combined.

3. Each of the two mixtures were spray dried using a suitable spray dryer (PSD-1) and the resulting spray dried products were dried using a tray oven dryer and collected.

TABLE 2
Ingredients for the preparation of the solid
dispersions SD1 Powder and SD2 Powder

Quantity per Batch
(gram per 1000 g SD)

| | SD1a/b | SD2a/b |
|---|---|---|
| Component | Powder | Powder |
| Compound (a) | 333.3 | 333.3 |
| Eudragit L100 | 666.7 | NA |
| Hydroxypropyl methylcellulose E5 | NA | 666.7 |
| Methanol^{a} | 14384.6 | 7107.5 |
| Methylene Chloride^{a} | NA | 11890.6 |
| Total batch size | 1000 | 1000 |

^{a}Removed (by drying) during processing

A single 400-mg dose of Compound (a) for each formulation concept (oral solution, SF1, and SF2) was administered to determine its relative oral bioavailability. 32 healthy adult participants (male and female, 18 to 55 years of age) were enrolled in this study.

The volunteers were divided in two panels; in each panel, a different solid formulation was compared to the oral solution. The two panels were organized as follows:

Panel 1 (n=16):
Treatment A: single 400-mg dose (2.5 mL of a 160-mg/mL oral solution) of Compound (a) administered as oral solution in fasted conditions.
Treatment B: single 400-mg dose (2×200-mg tablets) of Compound (a) administered as solid formulation #1b (SF1b) in fasted conditions.
Treatment A and B (treatment sequence group 1 [n=8]: AB; treatment sequence group 2 [n=8]: BA).
   Panel 2 (n=16):
Treatment A: single 400-mg dose (2.5 mL of a 160-mg/mL oral solution) of Compound (a) administered as oral solution in fasted conditions.
Treatment C: single 400-mg dose (2×200-mg tablets) of Compound (a) administered as solid formulation #2b (SF2b) in fasted conditions.
Treatments A and C (treatment sequence group 1 [n=8]: AC; treatment sequence group 2 [n=8]: CA).

Tablets were administered orally, together with approximately 240 mL of non-carbonated water, in fasted conditions. In the fasted state, participants did not consume food for at least 10 hours before study drug intake. Intake of water was not allowed from 2 hours before and until 2 hours after study drug intake, except for the water used for study drug intake.

During two treatment periods, each participant received 2 treatments randomized according to a classical 2-sequence, 2-period Williams design. Intake of Compound (a) in subsequent treatment periods in an individual participant was separated by a washout period of at least 35 days. The day of study drug intake in Treatment Period 1 (Day 1) was the first day of the washout period. The last day of the washout period of Treatment Period 1 (Day 35) may have coincided with Day-1 of Treatment Period 2.

Venous blood samples were collected for measurement of plasma concentrations of Compound (a) at time points as indicated in Table 3. The collected plasma samples were analyzed using a fully validated liquid chromatography-mass spectrometry/mass spectrometry (LC-MS/MS) method, described in further detail below.

### Lc-MS/MS Method Used for Plasma Sample Analysis

Sample processing was performed by means of protein precipitation using a sample volume of 25.0 μL plasma to which 20 μL of methanol was added containing 50.0 ng/ml of a stable isotopic labelled internal standard of Compound (a) was added followed by the addition of 200 μL acetonitrile. After vortex-mixing for 1 min at 1200 rpm and room temperature, the sample extract was centrifuged for 5 min at approximately 3500×g and 20° C. 100 μL of the supernatant was then transferred to a 1.2 mL polypropylene deep well plate using a liquidator, and 50 μL water was added. The plate was capped with a silicone mat and vortex-mixed for 30 seconds at 1200 rpm and room temperature. The sample extract was then transferred to an UHPLC autosampler (Agilent 1290 Infinity II) for loading to the LC-MS/MS analyzer.

Separation between metabolites and interfering endogenous compounds was achieved by injecting 20 μL sample extract onto an Acquity UPLC BEH C18 column (50×2.1 mm, 1.7 μm, Waters) at 50° C. and using 10 mM ammonium bicarbonate solution as mobile phase A, and methanol: acetonitrile (50:50, v/v) as mobile phase B. A gradient elution at a flow rate of 1.00 mL/min was applied using the following program: 35% A and 65% B were held constant for the first 1 min, followed by an increase to 90% B at 1.01 min, which was held constant up to 1.50 min. At 1.51 min the eluents were set back to their initial conditions to re-equilibrate the column.

A API4000 triple quadrupole mass spectrometer (Sciex) equipped with a turbo ion spray source was used for analyte detection in positive ion mode using electrospray ionization. The mass spectrometer was operated under the following conditions: Gas 1 (nebulizer gas) flow (N₂), 45 arbitrary units; Gas 2 (heater gas) flow (N₂), 45 arbitrary units; ion source voltage 4.5 kV; capillary temperature 500° C.; and maximum ion injection time of 150 ms. Curtain and CAD gas (N₂) was 30 L/min and 6 L/min, respectively. The mass spectrometer was optimized for the quantification of Compound (a) (MRM transition m/z 583.1>354.1) and the stable isotope labeled internal standard (MRM transition m/z 591.2>354.1).

A 9-point calibration curve ranging from 0.500 to 500 ng/ml Compound (a) was prepared in blank human EDTA plasma. Quality control (QC) samples at 1.50, 25.0, 250 and 400 ng/mL were prepared from separate analyte stock solutions that was spiked to blank human EDTA plasma and were stored under similar conditions as the study samples. Calibration standards, QC and study samples were processed at the same time.

A linear regression model with 1/x2 weighing was used, peak area ratios of the analyte to its internal standard were plotted against the analyte concentrations. The concentrations in samples were calculated by interpolation from the standard curve.

A high range assay was also developed and validated for the analysis of higher analyte concentrations applying a similar sample preparation and LC-MS/MS analysis method. Therefore, a 9-point calibration curve ranging from 5.00 to 5000 ng/ml of Compound (a) was prepared and QC samples at 15.0, 1500 and 3750 ng/ml analyte concentrations in Blank human EDTA plasma.

TABLE 3
Blood sample collection intervals

| | Time of sample collection |
|---|---|
| Day of study | (hours after dosing) |
| Day 1 | +1 |
| | +2 |
| | +3 |
| | +4 |
| | +5 |
| | +6 |
| | +7 |
| | +8 |
| | +10 |
| | +12 |
| | +16 |
| Day 2 | +24 |
| | +36 |
| Day 3 | +48 |
| Day 4 | +72 |
| Day 5 | +96 |
| Day 6 | +120 |
| Day 9 | +192 |
| Day 12 | +264 |
| Day 15 | +336 |
| Day 22 | +504 |
| Day 28 | +648 |
| Day 36 | +840 |

For all treatments, descriptive statistics were calculated for plasma concentrations of Compound (a) at each applicable time point specified, and for the derived Pharmacokinetic (PK) parameters. Statistics include sample size (n), mean, standard deviation (SD), % CV ((percentage) coefficient of variation), geometric mean, median, minimum, and maximum. The non-compartmental PK analysis was done, using the validated computer program Phoenix™ WinNonlin® (Copyright 1998-2017, Certara L. P., USA). SAS (SAS Institute Inc., Cary, NC, USA) was used, predominantly for the creation of PK tables and figures, for the estimation of the treatment ratios of PK parameters and for the statistical evaluation of the PK data. Plasma concentrations and timepoints of extraction were inserted for every subject. Rsq more than 0.9; Extrapolation to estimate AUCinf should be <20%. The following Pharmacokinetic (PK) parameters, were measured:
Cₘₐₓ maximum observed plasma analyte concentration;
tₘₐₓ the actual sampling time to reach the maximum observed plasma analyte concentration (i.e., time at which the maximum concentration (Cₘₐₓ) is observed);
AUCₗₐₛₜ AUC from time 0 to the time of the last measurable (non-below quantification limit (BQL)) concentration, calculated by linear-linear trapezoidal summation;
AUC_{∞} AUC from time 0 to infinity, calculated as AUCₗₐₛₜ+C_{last/λz}, where Cₗₐₛₜ is the last observed measurable (non-BQL) concentration; extrapolations of more than 20.00% of the total AUC are reported as approximations. t_{1/2 term} apparent terminal elimination half-life, calculated as 0.693/λ_{z};
Δ_{z} apparent terminal elimination rate constant, estimated by linear regression using the terminal log-linear phase of the log transformed plasma analyte concentration versus time curve;

The following requirements were met for an acceptable calculation of t_{1/2term}, λ_{z}, AUC_{∞}, and related parameters:
A) at least 3 data points were used in the calculation;
B) coefficient of determination (r²_{adj}) was at least 0.900.

Dose-normalization was done by dividing the relevant PK parameter by the dose (D).

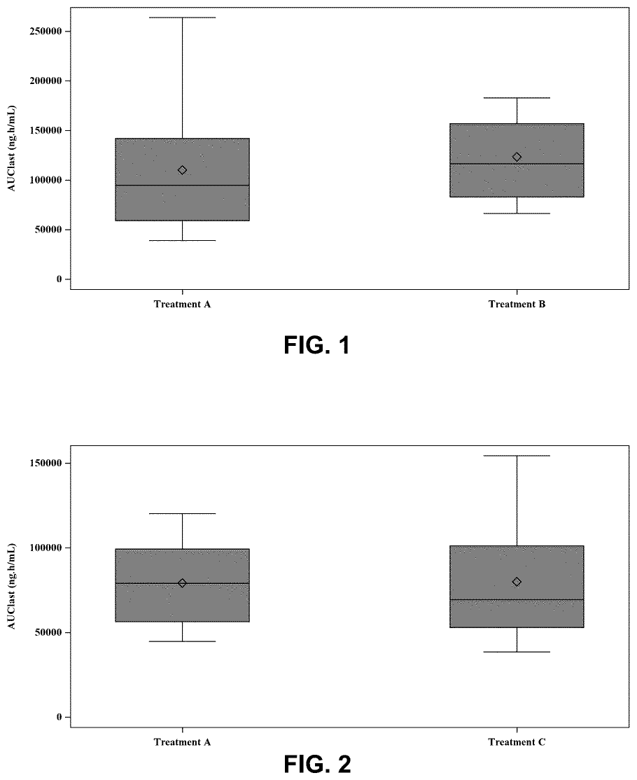

The PK results for SF1b oral tablets comprising 200 mg of Compound (a) demonstrated increased bioavailability compared to the oral solution formulation, with a relative bioavailability of (Geometric mean ratio (GMR) with 90% Confidence Interval (CI)) 137.96% (106.24%-179.16%) for Cₘₐₓ, 122.77% (103.83%-145.16%) for AUCₗₐₛₜ and 123.70% (104.68%-146.17%) for AUC_{∞}. For the GMR calculation, the least square (LS) means of the log-transformed primary PK parameters for each treatment was estimated with a single linear mixed effects model, controlling for treatment (oral solution fasted, solid dose formulation fasted, solid dose formulation fed), sequence and period as fixed effects, and participant as a random effect. A 90% CI was constructed around the difference between the LS means of test and reference for each comparison. Both the difference between the LS means and the 90% CIs were retransformed to the original scale. The relative oral bioavailability of Compound (a) was 23% higher for AUCₗₐₛₜ and 43% higher for Cₘₐₓ when administered as SF1b compared to oral solution, in fasted condition (FIG. 1).

The PK results for SF2b oral tablets comprising 200 mg of Compound (a) demonstrated similar bioavailability compared to the oral solution formulation, with a relative bioavailability of (GMR with 90% CI) 106.40% (91.03%-124.36%) for Cₘₐₓ, 97.60% (83.65%-113.89%,) for AUCₗₐₛₜ and 99.62% (85.96%-115.43%) for AUC_{∞}. The GMR and CI was calculated as described above. The relative oral bioavailability of Compound (a) was similar when administered as SF2b compared to oral solution, in fasted condition (FIG. 2).

Safety and tolerability of Compound (a) was evaluated throughout the study, by recording the adverse events (AE) and their grade, as reported by the participants.

An AE is any untoward medical occurrence in a subject administered the pharmaceutical formulation comprising Compound (a). An AE does not necessarily have a causal relationship with the pharmaceutical formulation comprising Compound (a). An AE can therefore be any unfavorable and unintended sign (including an abnormal finding), symptom, or disease temporally associated with the use of the pharmaceutical formulation comprising Compound (a), whether or not related to the administration of the pharmaceutical formulation comprising Compound (a) (definition per International Conference on Harmonisation [ICH].)

This includes any occurrence that is new in onset or aggravated in severity or frequency from the baseline condition, or abnormal results of diagnostic procedures, including laboratory test abnormalities.

Grade refers to the severity of the AE, according to the below guideline:
Grade 1 Mild: transient or mild discomfort (<48 hours); no medical intervention/therapy required.
Grade 2 Moderate: mild to moderate limitation in activity; some assistance may be needed; no or minimal medical intervention/therapy required.
Grade 3 Severe: marked limitation in activity; some assistance usually required; medical intervention/therapy required; hospitalizations possible.
Grade 4 Potentially Life-threatening: Extreme limitation in activity; significant assistance required; significant medical intervention/therapy required; hospitalization or hospice care probable.

All reported AEs with onset during the treatment phase and AEs that have worsened since baseline (ie, treatment-emergent AEs (TEAEs)) were included in the analysis.

### Overall Reported Events for Panels 1 and 2:

11 Grade 1 Treatment Emergent Events.
7 Grade 2 Treatment Emergent Events.
One Grade 3 Treatment Emergent Event.
No Grade 4 Treatment Emergent Events.

### Related Adverse Events (AEs) Reported

Panel 1 (400 mg fasted: Treatment A and Treatment B):
   two Grade 1 AEs of throat irritation and dermatitis on one subject, and throat irritation and dermaturia in another subject.
   one Grade 2 AE of headache and dermatitis.
Panel 2 (400 mg fasted: Treatment A and Treatment C):
   two Grade 1 AEs of headache and dermatitis each in one subject.

### Example 2

SF2a/b was the selected formulation based on Example 1 and was assessed in this Example.

The effect of food on the pharmacokinetics (PK) of Compound (a) was assessed in healthy participants after a single oral dose of SF2a/b, described in Example 1, administered at different dose levels in a randomized single dose, 2-period crossover study.

The effect of food on the PK of Compound (a) was determined at 3 different dose levels (50 mg, 200 mg, and 800 mg) for a standardized breakfast; at one dose level (800 mg) for a high-fat, high-calorie breakfast; at two dose levels (200 mg and 800 mg) for a low-fat, low-calorie breakfast, as well as for a low-fat, high-calorie breakfast.

124 healthy adult participants (male and female, 18 to 55 years of age) were enrolled in this study. The volunteers were divided in six panels, which were organized as follows:
Panel 3, n=15:
Treatment D: A single 800-mg dose (4×200-mg tablets) of Compound (a) SF2b in fasted conditions.
Treatment E: A single 800-mg dose (4×200-mg tablets) of Compound (a) SF2b in fed conditions (standardized breakfast).
Treatments D and E (treatment sequence group 1 [n=8]: DE; treatment sequence group 2 [n=8]: ED).
   Panel 4, n=14:
Treatment F: A single 200-mg dose (1×200-mg tablet) of Compound (a) SF2b in fasted conditions.
Treatment G: A single 200-mg dose (1×200-mg tablet) of Compound (a) SF2b, in fed conditions (standardized breakfast).
Treatments F and G (treatment sequence group 1 [n=7]: FG; treatment sequence group 2 [n=7]: GF).
   Panel 5, n=13:
Treatment H: A single 50-mg dose (1×50-mg tablet) of Compound (a) SF2a in fasted conditions.
Treatment I: A single 50-mg dose (1×50-mg tablet) of Compound (a) SF2a in fed conditions (standardized breakfast).
Treatments H and I (treatment sequence group 1 [n=7]: HI; treatment sequence group 2 [n=7]: IH).
   Panel 6 (n=16):
Treatment J: single 800-mg dose (4×200-mg tablets) of Compound (a) administered as SF2b in fasted conditions.
Treatment K: single 800-mg dose (4×200-mg tablets) of Compound (a) administered as SF2b in fed conditions (high-fat, high-calorie breakfast).
Treatments J and K (treatment sequence group 1 [n=8]: JK; treatment sequence group 2 [n=8]: KJ).
   Panel 7 (n=32):
Treatment L: single 800-mg dose (4×200-mg tablets) of Compound (a) administered as SF2b in fed conditions (low-fat, low-calorie breakfast).
Treatment M: single 800-mg dose (4×200-mg tablets) of Compound (a) administered as SF2b in fed conditions (low-fat, high-calorie breakfast).
Treatment L (n=16) or Treatment M (n=16).
   Panel 8 (n=32):
Treatment N: single 200-mg dose (1×200-mg tablet) of Compound (a) administered as SF2b in fed conditions (low-fat, low-calorie breakfast).
Treatment O: single 200-mg dose (1×200-mg tablet) of Compound (a) administered as SF2b in fed conditions (low-fat, high-calorie breakfast).
Treatment N (n=16) or Treatment O (n=16).

Tablets were administered orally, together with approximately 240 mL of non-carbonated water, in fasted or fed conditions. In the fasted state, participants did not consume food for at least 10 hours before study drug intake.

On day 1 of all treatments in each panel, the participants had fasted overnight for at least 10 hours before study drug intake. Intake of water was not allowed from 2 hours before and until 2 hours after study drug intake, except for the water used for study drug intake and breakfast.

In case of intake in fed conditions, study drug was administered within 10 minutes after completing a standardized breakfast (Treatments E, G, and I), or a high-fat, high-calorie breakfast (Treatment K), a low-fat, low-calorie breakfast (Treatments L and N), or a low-fat, high-calorie breakfast (Treatments M and O).

For treatments E, G, and I a standardized breakfast was served, consisting of (or its equivalent) 4 slices of bread, 2 slices of ham or cheese, butter, jelly, and 1 or 2 cups (up to 355 mL) of decaffeinated coffee or tea with milk and/or sugar, if desired (containing approximately: fat: 21 g, carbohydrates: 67 g, proteins: 19 g; total calories: 533 kcal).

For treatment K in a high-fat, high-calorie breakfast was served. The high-fat, high-calorie breakfast consisted of (or its equivalent) 2 eggs fried in butter, 2 strips of bacon, 2 slices of toast with butter, 100 g of hashbrown potatoes (fried with butter), and 240 mL of whole milk (containing approximately: fat: 500-600 kcal, carbohydrates: 250 kcal, proteins: 150 kcal; total calories 900-1000 kcal).

For treatments L and N a low-fat, low-calorie breakfast was served, consisting of (or its equivalent) 2 slices of brown bread, 20 g jam, 125 mL skim milk, and 1 cup of decaffeinated coffee or tea without milk or sugar (containing approximately 247 kcal and 2.5 g fat).

For treatments M and O a low-fat, high-calorie breakfast was served, consisting of (or its equivalent) 4 slices of white bread, 20 g jam, 20 g honey, 330 mL fruit juice, 1 medium banana, 125 g low-fat fruit yogurt, 250 mL skim milk, and 1 cup of decaffeinated coffee or tea without milk or sugar (containing approximately 911 kcal and 4.2 g fat).

Within 10 minutes after completing the breakfast, but no more than 30 minutes after the start of breakfast, study drug was administered.

In Panels 3 to 6, during 2 treatment periods, each participant received 2 treatments randomized according to a classical 2-sequence, 2-period Williams design. Intake of Compound (a) in subsequent treatment periods in an individual participant was separated by a washout period of at least 35 days. The day of study drug intake in Treatment Period 1 (Day 1) was the first day of the washout period. The last day of the washout period of Treatment Period 1 (Day 35) may have coincided with Day-1 of Treatment Period 2. In Panels 7 and 8, each participant was randomized to receive only 1 of 2 treatments during 1 treatment period.

Venous blood samples were collected for measurement of plasma concentrations of Compound (a) at time points as indicated in Example 1. The collected plasma samples were analyzed using a LC-MS/MS method described in Example 1.

The PK parameters were calculated in the same manner as described in Example 1. Table 4A shows the plasma PK parameters of Treatment D, Table 4B shows the plasma PK parameters for Treatment E, Table 4C shows the plasma PK parameters for Treatment F, Table 4D shows the plasma PK parameters for Treatment G, Table 4E shows the plasma PK parameters for Treatment H and Table 4F shows the plasma PK parameters for Treatment I.

TABLE 4A
Plasma PK parameters of Treatment D

| | Cₘₐₓ | tₘₐₓ | AUCₗₐₛₜ | tₗₐₛₜ | AUC_{∞} | t_{1/2, term} |
|---|---|---|---|---|---|---|
| | μg/L | h | h*μg/L | h | h*μg/L | h |
| N | 12 | 12 | 12 | 12 | 12 | 12 |
| Mean | 1300 | NC | 148907 | NC | 160388 | 193.5 |
| SD | 614 | NC | 70982 | NC | 80993 | 75.9 |
| Min | 438 | 7.00 | 63449 | 840.00 | 64110 | 98.0 |
| Median | 1450 | 8.00 | 137787 | 840.00 | 143752 | 179.0 |
| Max | 2350 | 12.00 | 260545 | 840.00 | 292725 | 295.6 |
| CV % | 47.2 | NC | 47.7 | NC | 50.5 | 39.2 |
| Geo. | 1150 | NC | 133367 | NC | 141633 | 179.7 |
| Mean | | | | | | |

NC: not calculated.

Individual data and descriptive statistics are presented with 2 decimal places for tₘₐₓ and tₗₐₛₜ, with 1 decimal for t_{1/2,term}, rounded to integer for AUCs and with 3 significant digits for other parameters. CV % is presented with 1 decimal place.
Best-fit method was used for the estimation of half-lives.

TABLE 4B
Plasma PK parameters of Treatment E

| | Cₘₐₓ | tₘₐₓ | AUCₗₐₛₜ | tₗₐₛₜ | AUC_{∞} | t_{1/2, term} |
|---|---|---|---|---|---|---|
| | μg/L | h | h*μg/L | h | h*μg/L | h |
| N | 15 | 15 | 15 | 15 | 14 | 14 |
| Mean | 3500 | NC | 357150 | NC | 347619 | 175.5 |
| SD | 848 | NC | 131310 | NC | 92295 | 61.0 |
| Min | 2130 | 5.00 | 200503 | 504.00 | 202255 | 94.8 |
| Median | 3510 | 6.00 | 337042 | 840.00 | 344704 | 150.3 |
| Max | 4930 | 16.00 | 750680 | 840.00 | 549182 | 253.9 |
| CV % | 24.3 | NC | 36.8 | NC | 26.6 | 34.8 |
| Geo. | 3400 | NC | 339684 | NC | 336755 | 165.6 |
| Mean | | | | | | |

NC: not calculated, NE: Not estimable.

Individual data and descriptive statistics are presented with 2 decimal places for tₘₐₓ and tₗₐₛₜ, with 1 decimal for t_{1/2,term}, rounded to integer for AUCs and with 3 significant digits for other parameters. CV % is presented with 1 decimal place. Best-fit method was used for the estimation of half-lives.

TABLE 4C
Plasma PK parameters of Treatment F

| | Cₘₐₓ | tₘₐₓ | AUCₗₐₛₜ | tₗₐₛₜ | AUC_{∞} | t_{1/2, term} |
|---|---|---|---|---|---|---|
| | μg/L | h | h*μg/L | h | h*μg/L | h |
| N | 12 | 12 | 12 | 12 | 11 | 12 |
| Mean | 602 | NC | 64806 | NC | 62041 | 205.1 |
| SD | 191 | NC | 24752 | NC | 19044 | 114.1 |
| Min | 334 | 5.00 | 38612 | 840.00 | 39189 | 118.9 |
| Median | 580 | 7.50 | 59008 | 840.00 | 58457 | 174.1 |
| Max | 889 | 12.00 | 119878 | 840.00 | 104195 | 552.2 |
| CV % | 31.7 | NC | 38.2 | NC | 30.7 | 55.6 |
| Geo. | 573 | NC | 61065 | NC | 59598 | 188.0 |
| Mean | | | | | | |

NC: not calculated

Individual data and descriptive statistics are presented with 2 decimal places for tₘₐₓ and tₗₐₛₜ, with 1 decimal for t_{1/2, term}, rounded to integer for AUCs and with 3 significant digits for other parameters. CV % is presented with 1 decimal place. Best-fit method was used for the estimation of half-lives.

TABLE 4D
Plasma PK parameters of Treatment G

| | Cₘₐₓ | tₘₐₓ | AUCₗₐₛₜ | tₗₐₛₜ | AUC_{∞} | t_{1/2, term} |
|---|---|---|---|---|---|---|
| | μg/L | h | h*μg/L | h | h*μg/L | h |
| N | 12 | 12 | 12 | 12 | 12 | 12 |
| Mean | 996 | NC | 100317 | NC | 105882 | 198.5 |
| SD | 299 | NC | 28185 | NC | 30495 | 47.6 |
| Min | 682 | 5.00 | 75752 | 840.00 | 79436 | 141.9 |
| Median | 892 | 8.00 | 89500 | 840.00 | 95253 | 208.4 |
| Max | 1660 | 12.00 | 160157 | 840.00 | 165719 | 293.6 |
| CV % | 30.0 | NC | 28.1 | NC | 28.8 | 24.0 |
| Geo. | 961 | NC | 97220 | NC | 102425 | 193.4 |
| Mean | | | | | | |

NC: not calculated

Individual data and descriptive statistics are presented with 2 decimal places for tₘₐₓ and tₗₐₛₜ, with 1 decimal for t_{1/2,term}, rounded to integer for AUCs and with 3 significant digits for other parameters. CV % is presented with 1 decimal place.
Best-fit method was used for the estimation of half-lives.

TABLE 4E
Plasma PK parameters of Treatment H

| | Cₘₐₓ | tₘₐₓ | AUCₗₐₛₜ | tₗₐₛₜ | AUC_{∞} | t_{1/2, term} |
|---|---|---|---|---|---|---|
| | μg/L | h | h*μg/L | h | h*μg/L | h |
| N | 13 | 13 | 13 | 13 | 13 | 13 |
| Mean | 214 | NC | 19868 | NC | 20981 | 176.0 |
| SD | 98.5 | NC | 9274 | NC | 10859 | 60.1 |
| Min | 79.4 | 5.00 | 6412 | 504.00 | 6851 | 72.4 |
| Median | 208 | 7.00 | 18516 | 840.00 | 18838 | 162.4 |
| Max | 482 | 12.00 | 42729 | 840.00 | 49385 | 322.6 |
| CV % | 46.1 | NC | 46.7 | NC | 51.8 | 34.2 |
| Geo. | 195 | NC | 18033 | NC | 18806 | 166.6 |
| Mean | | | | | | |

Individual data and descriptive statistics are presented with 2 decimal places for tₘₐₓ and tₗₐₛₜ, with 1 decimal for t_{1/2,term}, rounded to integer for AUCs and with 3 significant digits for other parameters. CV % is presented with 1 decimal place.

Best-fit method was used for the estimation of half-lives.

TABLE 4F
Plasma PK parameters of Treatment I

| | Cₘₐₓ | tₘₐₓ | AUCₗₐₛₜ | tₗₐₛₜ | AUC_{∞} | t_{1/2, term} |
|---|---|---|---|---|---|---|
| | μg/L | h | h*μg/L | h | h*μg/L | h |
| N | 12 | 12 | 12 | 12 | 12 | 12 |
| Mean | 288 | NC | 22924 | NC | 24635 | 185.0 |
| SD | 160 | NC | 11896 | NC | 15384 | 74.4 |
| Min | 124 | 4.00 | 12201 | 648.00 | 12517 | 94.0 |
| Median | 267 | 6.50 | 20389 | 840.00 | 20763 | 159.2 |
| Max | 727 | 10.00 | 57880 | 840.00 | 71103 | 368.1 |
| CV % | 55.7 | NC | 51.9 | NC | 62.4 | 40.2 |
| Geo. | 257 | NC | 21062 | NC | 22068 | 173.6 |
| Mean | | | | | | |

NC: not calculated

Individual data and descriptive statistics are presented with 2 decimal places for tₘₐₓ and tₗₐₛₜ, with 1 decimal for t_{1/2,term}, rounded to integer for AUCs and with 3 significant digits for other parameters. CV % is presented with 1 decimal place.
Best-fit method was used for the estimation of half-lives. A 90% confidence interval (CI) was constructed around the difference between the least square (LS) means of test and reference for each comparison. Both the difference between the LS means and the 90% CIs were retransformed to the original scale.
Table 5A shows a comparison of the PK parameters obtained in Treatment E and Treatment D (corresponding to Panel 3). Table 5B shows a comparison of the PK parameters obtained in Treatment G and Treatment F (corresponding to Panel 4). Table 5C shows a comparison of the PK parameters obtained in Treatment I and Treatment H (corresponding to Panel 5). Table 5D shows a comparison of the PK parameters obtained in Treatment J and Treatment K (corresponding to Panel 6). Table 5E shows a comparison of the PK parameters obtained in Treatment G and Treatment N. Table SF shows a comparison of the PK parameters obtained in Treatment E and Treatment L. Table 5G shows a comparison of the PK parameters obtained in Treatment G and Treatment O. Table 5H shows a comparison of the PK parameters obtained in Treatment E and Treatment M.

TABLE 5A
Comparison of PK parameters of Treatment E and Treatment D

| | | | | | | | Treatment E | | | |
|---|---|---|---|---|---|---|---|---|---|---|
| Pharma- | | | | | | | vs Treatment D | | Intra- | Inter- |
| cokinetic | Geometric Mean | | | | | | Geometric | | subject | subject |
| Parameter | | Treat- | Treat- | P-values | | | Mean | 90% CI | variability | variability |
| (unit) | n | ment D | ment E | Treatment | Period | Sequence | Ratio (%) | (%) | CV (%) | CV (%) |
| Cₘₐₓ | (12; 15) | 1219 | 3385 | <0.0001 | 0.1305 | 0.6592 | 277.77 | 219.31- | 33.1 | 25.8 |
| (μg/L) | | | | | | | | 351.80 | | |
| AUCₗₐₛₜ | (12; 15) | 139557 | 336651 | <0.0001 | 0.0063 | 0.9912 | 241.23 | 209.14- | 18.9 | 34.7 |
| (h*μg/L) | | | | | | | | 278.25 | | |
| AUC_{∞} | (12; 14) | 147703 | 351698 | <0.0001 | 0.0180 | 0.9060 | 238.11 | 202.90- | 20.4 | 37.5 |
| (h*μg/L) | | | | | | | | 279.43 | | |

Pharmacokinetic parameters were log-transformed and submitted to a linear mixed effects model including treatment, sequence, period as fixed effects and participant as random effect.
n: (number of subjects with data for Treatment D; number of subjects with data for Treatment E).
CI: Confidence interval.
Geometric means are presented without decimals, p-values are presented with 4 decimal places, Geometric Mean Ratio (GMR) and 90% CI are presented with 2 decimal places.
CV: coefficient of variation.

TABLE 5B
Comparison of PK parameters of Treatment G and Treatment F

| | | | | | | | Treatment G | | | |
|---|---|---|---|---|---|---|---|---|---|---|
| Pharma- | | | | | | | vs Treatment F | | Intra- | Inter- |
| cokinetic | Geometric Mean | | | | | | Geometric | | subject | subject |
| Parameter | | Treat- | Treat- | P-values | | | Mean | 90% CI | variability | variability |
| (unit) | n | ment F | ment G | Treatment | Period | Sequence | Ratio (%) | (%) | CV (%) | CV (%) |
| Cₘₐₓ | (12; 12) | 559 | 984 | 0.0001 | 0.0631 | 0.0087 | 176.07 | 153.00- | 17.7 | 14.5 |
| (ug/L) | | | | | | | | 202.60 | | |
| AUCₗₐₛₜ | (12; 12) | 60522 | 101950 | <0.0001 | 0.3749 | 0.0620 | 168.45 | 149.01- | 15.2 | 24.1 |
| (h*μg/L) | | | | | | | | 190.43 | | |
| AUC_{∞} | (11; 12) | 60468 | 104004 | <0.0001 | 0.4519 | 0.1485 | 172.00 | 152.04- | 15.3 | 21.3 |
| (h*ug/L) | | | | | | | | 194.58 | | |

Pharmacokinetic parameters were log-transformed and submitted to a linear mixed effects model including treatment, sequence, period as fixed effects and participant as random effect.
n: (number of subjects with data for Treatment F; number of subjects with data for Treatment G).
CI: Confidence interval.
Geometric means are presented without decimals, p-values are presented with 4 decimal places, Geometric Mean Ratio (GMR) and 90% CI are presented with 2 decimal places.
CV: coefficient of variation.

TABLE 5C
Comparison of PK parameters of Treatment I and Treatment H

| | | | | | | | Treatment I | | | |
|---|---|---|---|---|---|---|---|---|---|---|
| Pharma- | | | | | | | vs Treatment H | | Intra- | Inter- |
| cokinetic | Geometric Mean | | | | | | Geometric | | subject | subject |
| Parameter | | Treat- | Treat- | P-values | | | Ratio (%) | 90% | variability | variability |
| (unit) | n | ment H | ment I | Treatment | Period | Sequence | Mean | CI (%) | CV (%) | CV (%) |
| Cₘₐₓ | (13; 12) | 193 | 257 | 0.0352 | 0.7887 | 0.2846 | 132.83 | 107.46- | 29.3 | 36.7 |
| (μg/L) | | | | | | | | 164.18 | | |
| AUCₗₐₛₜ | (13; 12) | 17940 | 21595 | 0.0853 | 0.8836 | 0.5367 | 120.38 | 100.94- | 23.9 | 39.8 |
| (h*μg/L) | | | | | | | | 143.55 | | |
| AUC_{∞} | (13; 12) | 18709 | 22683 | 0.0767 | 0.8560 | 0.5607 | 121.24 | 101.58- | 24.0 | 43.8 |
| (h*μg/L) | | | | | | | | 144.70 | | |

Pharmacokinetic parameters were log-transformed and submitted to a linear mixed effects model including treatment, sequence, period as fixed effects and participant as random effect.
n: (number of subjects with data for Treatment H; number of subjects with data for Treatment I).
CI: Confidence interval.
Geometric means are presented without decimals, p-values are presented with 4 decimal places, Geometric Mean Ratio (GMR) and 90% CI are presented with 2 decimal places.
CV: coefficient of variation.

TABLE 5D
Comparison of PK parameters of Treatment J and Treatment K

| | | | | Treatment K | | Source of |
|---|---|---|---|---|---|---|
| Pharma- | | | | vs Treatment J | | variability |
| cokinetic | Geometric LS Means | | | Geometric | | Intra- |
| Parameter | | Treat- | Treat- | Mean Ratio | 90% CI | subject |
| (unit) | n | ment J | ment K | (%) | (%) | CV (%) |
| Cₘₐₓ | (11; 16) | 991 | 4,052 | 409.01 | (289.37- | 46.2 |
| (ng/mL) | | | | | 578.11) | |
| AUCₗₐₛₜ | (11; 16) | 107,463 | 420,740 | 391.52 | (303.13- | 32.3 |
| (ng · h/mL) | | | | | 505.69) | |
| AUC_{∞} | (11; 15) | 109,760 | 421,565 | 384.08 | (299.41- | 31.4 |
| (ng · h/mL) | | | | | 492.69) | |

Pharmacokinetic parameters were log-transformed and submitted to a linear mixed effects model including treatment, sequence, period as fixed effects and participant as random effect.
n: (number of data for treatment J; number of data for treatment K).
CI: Confidence interval.
Geometric means are presented without decimals, p-values are presented with 4 decimal places, Geometric Mean Ratio (GMR) and 90% CI are presented with 2 decimal places.
CV: coefficient of variation.

TABLE 5E
Comparison of PK parameters of Treatment G and Treatment N

| | | | | Treatment N | | Source of |
|---|---|---|---|---|---|---|
| | | | | vs Treatment G | | variability |
| Pharma- | Geometric LS Means | | | Geometric | | Inter- |
| cokinetic | | Treat- | Treat- | Mean Ratio | 90% CI | participant |
| Parameter | n | ment G | ment N | (%) | (%) | CV (%) |
| Cₘₐₓ | (12; 14) | 961 | 697 | 72.54 | (58.69- | 32.3 |
| (ng/mL) | | | | | 89.65) | |
| AUCₗₐₛₜ | (11; 13) | 98,158 | 71,930 | 73.28 | (58.73- | 32.3 |
| (ng · h/mL) | | | | | 91.43) | |
| AUC_{∞} | (11; 13) | 103,038 | 75,844 | 73.61 | (58.64- | 33.2 |
| (ng · h/mL) | | | | | 92.40) | |

Pharmacokinetic parameters were log-transformed and submitted to a linear mixed effects model including treatment, sequence, period as fixed effects and participant as random effect.
n: (number of data for treatment G; number of data for treatment N).
CI: Confidence interval.
Geometric means are presented without decimals, p-values are presented with 4 decimal places, Geometric Mean Ratio (GMR) and 90% CI are presented with 2 decimal places.
CV: coefficient of variation.

TABLE 5F
Comparison of PK parameters of Treatment E and Treatment L

| | | | | Treatment L | | Source of |
|---|---|---|---|---|---|---|
| | | | | vs Treatment E | | variability |
| Pharma- | Geometric LS Means | | | Geometric | | Inter- |
| cokinetic | | Treat- | Treat- | Mean Ratio | 90% CI | participant |
| Parameter | n | ment E | ment L | (%) | (%) | CV (%) |
| Cₘₐₓ | (15; 13) | 3,400 | 2,059 | 60.56 | (51.68- | 24.9 |
| (ng/mL) | | | | | 70.98) | |
| AUCₗₐₛₜ | (13; 12) | 347,353 | 209,701 | 60.37 | (47.16- | 37.2 |
| (ng · h/mL) | | | | | 77.28) | |
| AUC_{∞} | (12; 13) | 341,395 | 220,909 | 64.71 | (50.74- | 36.6 |
| (ng · h/mL) | | | | | 82.52) | |

Pharmacokinetic parameters were log-transformed and submitted to a linear mixed effects model including treatment, sequence, period as fixed effects and participant as random effect.
n: (number of data for treatment E; number of data for treatment L).
CI: Confidence interval.
Geometric means are presented without decimals, p-values are presented with 4 decimal places, Geometric Mean Ratio (GMR) and 90% CI are presented with 2 decimal places.
CV: coefficient of variation.

TABLE 5G
Comparison of PK parameters of Treatment G and Treatment O

| | | | | Treatment O | | Source of |
|---|---|---|---|---|---|---|
| | | | | vs Treatment G | | variability |
| Pharma- | Geometric LS Means | | | Geometric | | Inter- |
| cokinetic | | Treat- | Treat- | Mean Ratio | 90% CI | participant |
| Parameter | n | ment G | ment O | (%) | (%) | CV(%) |
| Cₘₐₓ | (12; 14) | 961 | 700 | 72.81 | (62.15- | 23.9 |
| (ng/mL) | | | | | 85.31) | |
| AUCₗₐₛₜ | (11; 13) | 98,158 | 65,248 | 66.47 | (55.29- | 26.6 |
| (ng · h/mL) | | | | | 79.92) | |
| AUC_{∞} | (11; 13) | 103,038 | 69,274 | 67.23 | (55.35- | 28.2 |
| (ng · h/mL) | | | | | 81.66) | |

Pharmacokinetic parameters were log-transformed and submitted to a linear mixed effects model including treatment, sequence, period as fixed effects and participant as random effect.
n: (number of data for treatment G; number of data for treatment O).
CI: Confidence interval.
Geometric means are presented without decimals, p-values are presented with 4 decimal places, Geometric Mean Ratio (GMR) and 90% CI are presented with 2 decimal places. CV: coefficient of variation.

TABLE 5H
Comparison of PK parameters of Treatment E and Treatment M

| | | | | Treatment M | | Source of |
|---|---|---|---|---|---|---|
| | | | | vs Treatment E | | variability |
| Pharma- | Geometric LS Means | | | Geometric | | Inter- |
| cokinetic | | Treat- | Treat- | Mean Ratio | 90% CI | participant |
| Parameter | n | ment E | ment M | (%) | (%) | CV(%) |
| Cₘₐₓ | (15; 13) | 3,400 | 2,065 | 60.73 | (49.47- | 32.6 |
| (ng/mL) | | | | | 74.57) | |
| AUCₗₐₛₜ | (13; 13) | 347,353 | 226,709 | 65.27 | (52.57- | 33.1 |
| (ng · h/mL) | | | | | 81.03) | |
| AUC_{∞} | (12; 13) | 341,395 | 236,760 | 69.35 | (56.31- | 31.1 |
| (ng · h/mL) | | | | | 85.41) | |

Pharmacokinetic parameters were log-transformed and submitted to a linear mixed effects model including treatment, sequence, period as fixed effects and participant as random effect.
n: (number of data for treatment E; number of data for treatment M).
CI: Confidence interval.
Geometric means are presented without decimals, p-values are presented with 4 decimal places, Geometric Mean Ratio (GMR) and 90% CI are presented with 2 decimal places.
CV: coefficient of variation.

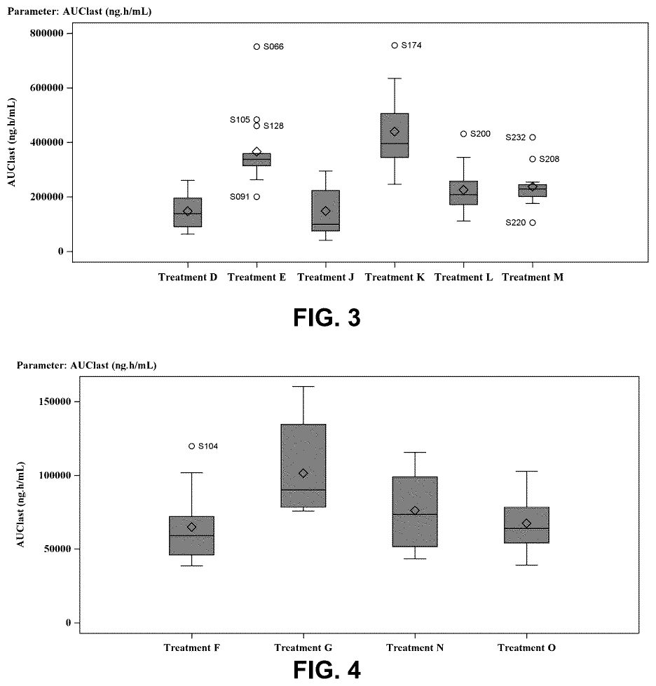

The intake of Compound (a), formulated as SF2a/b, with a standardized breakfast increased the bioavailability of Compound (a) compared to fasted condition (FIGS. 3 and 4). The increased food effect was more pronounced with higher dose with a relative bioavailability ranging from 124% (at 50 mg dose) to 243% (at 800 mg dose) for AUCₗₐₛₜ and from 133% (at 50 mg dose) to 278% (at 800 mg dose) for Cₘₐₓ.

The exposure to Compound (a) was approximately 4 times higher following single dose administration of Compound (a), formulated as SF2a/b, at 800 mg with high-fat, high-calorie breakfast compared to fasted conditions (FIG. 3).

The intake of Compound (a), formulated as SF2a/b, with low-fat, low-calorie breakfast significantly decreased exposure compared to intake following standardized breakfast, with a relative oral bioavailability ranging from 60% to 65% for the 800 mg dose and 73% to 74% for the 200 mg dose for Cₘₐₓ, AUCₗₐₛₜ and AUC_{∞} (FIG. 3).

The intake of Compound (a), formulated as SF2a/b, with low-fat, high-calorie breakfast significantly decreased exposure compared to intake following standardized breakfast, with a relative oral bioavailability ranging from 61% to 69% for the 800 mg dose and 66% to 73% for the 200 mg dose for Cₘₐₓ, AUCₗₐₛₜ and AUC_{∞} (FIGS. 3 and 4). The taste and overall acceptability of intake of Compound (a), formulated as SF2a/b was assessed during the study, using a taste acceptability questionnaire.

Overall acceptability was rated as “good to take” if the study intervention was a medicine for daily intake for a long period by 7 (87.5%) participants after receiving Treatment J (800 mg, fasted), and 6 (75.0%) participants after receiving Treatment K (800 mg, high-fat, high-calorie).

Safety and tolerability of Compound (a) was evaluated throughout the study, as described in Example 1.

The incidence of all TEAEs reported during the study was similar between the study intervention groups. Safety data suggests that Compound (a) was safe and well tolerated.

### Under Fasted Condition:

Overall, under fasted condition, at least 1 TEAE was reported in:

| | Reference | # Participants (%) |
|---|---|---|
| Panel 3 | Treatment D (800 mg, fasted) | 9 (69.2%) |
| Panel 4 | Treatment F (200 mg, fasted) | 6 (46.2%) |
| Panel 5 | Treatment H (50 mg, fasted) | 5 (38.5%) |

None of the TEAEs were considered related to the study intervention after dosing under fasted conditions in Panel 3 (Treatment D), Panel 4 (Treatment F) or Panel 5 (Treatment H).

### Under Fed Condition (Standardized Breakfast):

Overall, under fed condition, at least 1 TEAE was reported in:

| | Test | # Participants (%) |
|---|---|---|
| Panel 3 | Treatment E (800 mg, fed) | 6 (40.0%) |
| Panel 4 | Treatment G (200 mg, fed) | 7 (58.3%) |
| Panel 5 | Treatment I (50 mg, fed) | 8 (66.7%) |

None of the TEAEs were considered related to Treatment E, Treatment G or Treatment I, except for 1 (6.7%) participant after receiving Treatment E (ie, abdominal discomfort (grade 1)), and 1 (8.3%) participant after receiving Treatment G (ie, diarrhea (grade 1)).

### Example 3

A pharmaceutical formulation comprising Compound (a) is administered as an oral dosage form, to adult participants, in a fed state. Two dose regimens are applied:
Low dose: 150 mg of Compound (a) twice a day (BID) for two days (loading dose) and then 50 mg once a day (QD) for 26 days (maintenance dose);
High dose: 400 mg of Compound (a) twice a day (BID) for two days (loading dose) and then 150 mg once a day (QD) for 26 days (maintenance dose).

### Example 4

A pharmaceutical formulation comprising Compound (a) is administered as an oral dosage form, to adult participants, in a fed or a fasted state. Four dose regimens are applied:
Low dose (weekly): 150 mg of Compound (a) twice a day (BID) for two days, e.g., on day 1 (D1) and on day 2 (D2) (loading dose) and then 300 mg once a week (Q7D), e.g., on day 3 (D3), day 10 (D10), day 17 (D17), and on day 24 (D24) (maintenance dose);
Low dose (biweekly): 150 mg of Compound (a) twice a day (BID) for two days, e.g., on day 1 (D1) and on day 2 (D2), (loading dose) and then 150 mg Q3D and another 150 mg Q4D, e.g., on day 3 (D3), on day 6 (D6), on day 10 (D10), on day 13 (D13), on day 17 (D17), on day 20 (D20), on day 24 (D24), and on day 27 (D27) (or vice versa, e.g., Q4D followed by Q3D), for a total of 300 mg each week administered through two doses of 150 mg each (maintenance dose);
High dose (weekly): 450 mg of Compound (a) twice a day (BID) for two days, e.g., on day 1 (D1) and on day 2 (D2), (loading dose) and then 900 mg once a week (Q7D), e.g., on day 3 (D3), day 10 (D10), day 17 (D17), and on day 24 (D24) (maintenance dose);
High dose (biweekly): 900 mg of Compound (a) twice a day (BID) for two days, e.g., on day 1 (D1) and on day 2 (D2), (loading dose) and then 450 mg Q3D and another 450 mg Q4D, e.g., on day 3 (D3), on day 6 (D6), on day 10 (D10), on day 13 (D13), on day 17 (D17), on day 20 (D20), on day 24 (D24), and on day 27 (D27) (or vice versa, e.g., Q4D followed by Q3D), for a total of 900 mg each week administered through two doses of 450 mg each (maintenance dose)

### Example 5

A pharmaceutical formulation comprising Compound (a) as described in Example 2 (SF2a/b) was administered as an oral dosage form, to healthy adult participants, in a fed state. Four dose regimens were applied:
Panel I (weekly dosing): 450 mg of Compound (a) twice a day (BID) for two days: on day 1 (D1) and on day 2 (D2) (loading dose); and then 900 mg once a week (Q7D), on day 3 (D3), day 10 (D10), day 17 (D17), and on day 24 (D24) (maintenance dose).
Panel II (biweekly dosing): 450 mg of Compound (a) twice a day (BID) for two days: on day 1 (D1) and on day 2 (D2); (loading dose) and then 450 mg once every 3 (Q3D) or 4 days (Q4D): on day 3 (D3), on day 6 (D6), on day 10 (D10), on day 13 (D13), on day 17 (D17), on day 20 (D20), on day 24 (D24), and on day 27 (D27), for a total of 900 mg each week administered through two doses of 450 mg each (maintenance dose).
Panel III (weekly dosing): 150 mg of Compound (a) twice a day (BID) for two days: on day 1 (D1) and on day 2 (D2) (loading dose); and then 300 mg once a week (Q7D): on day 3 (D3), day 10 (D10), day 17 (D17), and on day 24 (D24) (maintenance dose).
Panel IV (biweekly dosing): 150 mg of Compound (a) twice a day (BID) for two days: on day 1 (D1) and on day 2 (D2) (loading dose); and then 150 mg Q3D or Q4D: on day 3 (D3), on day 6 (D6), on day 10 (D10), on day 13 (D13), on day 17 (D17), on day 20 (D20), on day 24 (D24), and on day 27 (D27), for a total of 300 mg each week administered through two doses of 150 mg each (maintenance dose).

Oral dosage forms were administered together with approximately 240 mL of non-carbonated water under fed conditions, and were taken within 10 minutes after completion of normal standardized normal fat breakfast in the morning or after completion of normal standardized normal fat dinner in case of evening dosing. Intake of Compound (a) took place at around the same time each day. Intake of Compound (a) marked time 0 hours.

A standardized normal fat meal was served approximately 30 minutes before study drug intake. The standardized normal fat meal contained approximately 21 g of fat, 67 g of carbohydrates, 19 g of proteins resulting in around 533 kcal (an example meal is 4 slices of bread, 2 slices of ham or cheese, butter, jelly, and 1 or 2 cups (up to 355 mL) of decaffeinated coffee or tea with milk and/or sugar). The standardized normal fat meal was ingested entirely within 30 minutes or less. Within 10 minutes after completing the meal, but no more than 30 minutes after the start of the meal, study drug was administered. In case of Compound (a) intake in the evening, timing of standardized dinner was aligned to time of drug intake.

Venous blood samples were collected for measurement of plasma concentrations of Compound (a) at time points as indicated in Table 6, except for Study Panel I. The collected plasma samples were analyzed using a LC-MS/MS method described in Example 1.

For Study Panel I, capillary whole blood samples was collected using the TASSO-M20 device for the determination of Compound (a) capillary whole blood concentrations on Days 1, 24, 25, 26, 30, 38, 40, 45, 51 and 59. Capillary whole blood samples collected were be compared directly to the venous whole blood samples which were collected at the same timepoints.

For Study Panel 1 only, additional whole blood samples were be collected on Days 1, 24, 25, 26, 38, 45, 51, and 59 using venipuncture. The whole blood samples were be used for the determination of Compound (a) whole blood concentrations and for determination of Compound (a) plasma concentrations. To determine Compound (a) venous whole blood concentrations, an aliquot of the whole blood sample was be pipetted onto the TASSO M-20 for better comparison with capillary whole blood samples collected by TASSO M-20.

Capillary whole blood and venous whole blood samples collected using the TASSO-M20 device, collects 4 volumetric blood sample tips. Bioanalytical sample processing was performed after sample extraction and protein precipitation followed by LC-MS/MS analysis using a fully validated method. Therefore, 1 blood sample tip that corresponds to 20 μL dried human whole blood was transferred to a Micronics tube and 200 μL ethyl acetate containing 2% formic acid followed by 200 μL of a methanol solution containing 100 ng/ml of the stable isotopic labelled internal standard (D613C2-labelled Compound (a)) were added. Sample extraction was performed by shaking the tubes at 1000 rpm at 20° C. for 120 min using a Thermomixer. Afterwards, 50 μL of the sample extract was transferred to a 96 round deep-well microplate (Porvair), to which 450 μL methanol was added. The plate was capped with a silicone mat and vortex-mixed, followed by injection to a SIL-30 AC MP autosampler (Shimadzu) for loading to the LC-MS/MS analyzer.

Separation between metabolites and interfering endogenous compounds was achieved by injecting 2 μL sample extract onto an Acquity UPLC BEH C18 column (50×2.1 mm, 1.7 μm, Waters) at 50° C. and using 10 mM ammonium bicarbonate solution as mobile phase A, and methanol: acetonitrile (50:50, v/v) as mobile phase B. A gradient elution at a flow rate of 0.600 mL/min was applied using the following program: 35% B was increased to 90% B in 3 min, was further increased to 98% B at 3.01 min and held constant up to 4.00 min. At 4.01 min the eluents were set back to their initial conditions to re-equilibrate the column until 5.0 min.

A Triple Quad 6500+ mass spectrometer (Sciex) equipped with a turbo ion spray source was used for analyte detection in positive ion mode using electrospray ionization. The mass spectrometer was operated under the following conditions: Gas 1 (nebulizer gas) flow (N₂), 40 arbitrary units; Gas 2 (heater gas) flow (N₂), 50 arbitrary units; ion source voltage 4.5 kV; capillary temperature 500° C. Curtain and CAD gas (N₂) was 35 L/min and 6 L/min, respectively. The mass spectrometer was optimized for the quantification of Compound (a) (MRM transition m/z 583.1>354) and the stable isotope labeled internal standard (MRM transition m/z 591.1>354) with a dwell time of 75 ms, respectively.

A 10-point calibration curve ranging from 5.00 to 5000 ng/mL Compound (a) was prepared in blank human blood, and the TASSO-M20 device was filled by slowly pipetting ca. 150 μL of each of the whole human blood standards into individual devices until blood was visible at the bottom in the overfill compartment. Quality control (QC) samples at 15.0, 150, 2000 and 4000 ng/mL were prepared from separate analyte stock solutions that was spiked to blank human blood, and filled similarly into individual TASSO-M20 devices, which were stored under similar conditions as study samples. The clear film at the back of the TASSO-M20 device was peeled-off to expose the sample tip vent. The TASSO-M20 devices were placed in a storage bag containing desiccant bag(s), and stored at room temperature for minimum 2 h, ideally overnight or longer in a cupboard until sample extraction was performed.

Calibration standards, QC and study samples were processed at the same time.

A linear regression model with 1/x² weighing was used, peak area ratios of the analyte to its internal standard were plotted against the analyte concentrations. The concentrations in samples were calculated by interpolation from the standard curve.

TABLE 6
Blood sample collection intervals

| | Panels I and III | Panels II and IV |
|---|---|---|
| | Sampling time (hours) | Sampling time (hours) |
| | Compound (a) Intake | Compound (a) Intake |
| Day of study | was at 0 hours | was at 0 hours |
| Day 1 | Predose | Predose |
| | +1 | +1 |
| | +3 | +3 |
| | +5 | +5 |
| | +6 | +6 |
| | +7 | +7 |
| | +8 | +8 |
| | +10 | +10 |
| | +12 | +12 |
| Day 2 | Predose | Predose |
| | +1 | +1 |
| | +3 | +3 |
| | +5 | +5 |
| | +6 | +6 |
| | +7 | +7 |
| | +8 | +8 |
| | +10 | +10 |
| | +12 | +12 |
| Day 3 | Predose | Predose |
| | +1 | +1 |
| | +3 | +3 |
| | +5 | +5 |
| | +6 | +6 |
| | +7 | +7 |
| | +8 | +8 |
| | +10 | +10 |
| | +12 | +12 |
| Day 4 | +16 | +16 |
| | +24 | +24 |
| | +36 | +36 |
| Day 5 | +48 | +48 |
| Day 6 | +72 | Predose |
| | | +8 |
| Day 10 | Predose | Predose |
| | +8 | +8 |
| Day 13 | — | Predose |
| | | +8 |
| Day 17 | Predose | Predose |
| | +8 | +8 |
| Day 20 | — | Predose |
| | | +8 |
| Day 24 | Predose | Predose |
| | +1 | |
| | +3 | |
| | +5 | |
| | +6 | +8 |
| | +7 | |
| | +8 | |
| | +10 | |
| | +12 | |
| Day 25 | +16 | — |
| | +24 | |
| | +36 | |
| Day 26 | +48 | — |
| Day 27 | +72 | Predose |
| | | +1 |
| | | +3 |
| | | +5 |
| | | +6 |
| | | +7 |
| | | +8 |
| | | +10 |
| | | +12 |
| Day 28 | +96 | +16 |
| | | +24 |
| | | +36 |
| Day 29 | — | +48 |
| Day 30 | +144 | +72 |
| Day 31 | +168 | +96 |
| Day 34 | — | +168 |
| Day 38 | +336 | — |
| Day 40 | +384 | — |
| Day 41 | — | +336 |
| Day 45 | +504 | — |
| Day 48 | — | +504 |
| Day 51 | +648 | — |
| Day 54 | — | +648 |
| Day 59 | +840 | — |
| Day 62 | — | +840 |

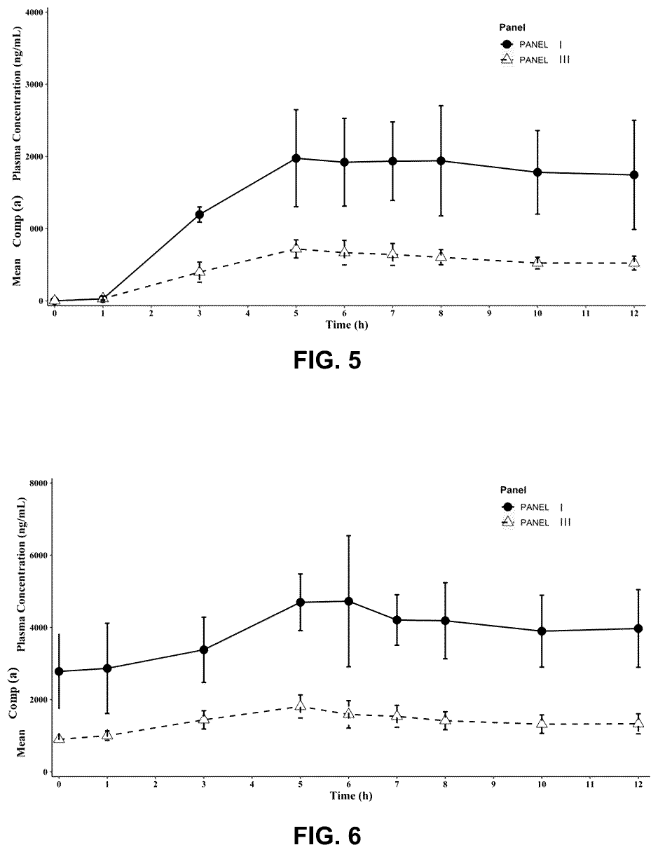

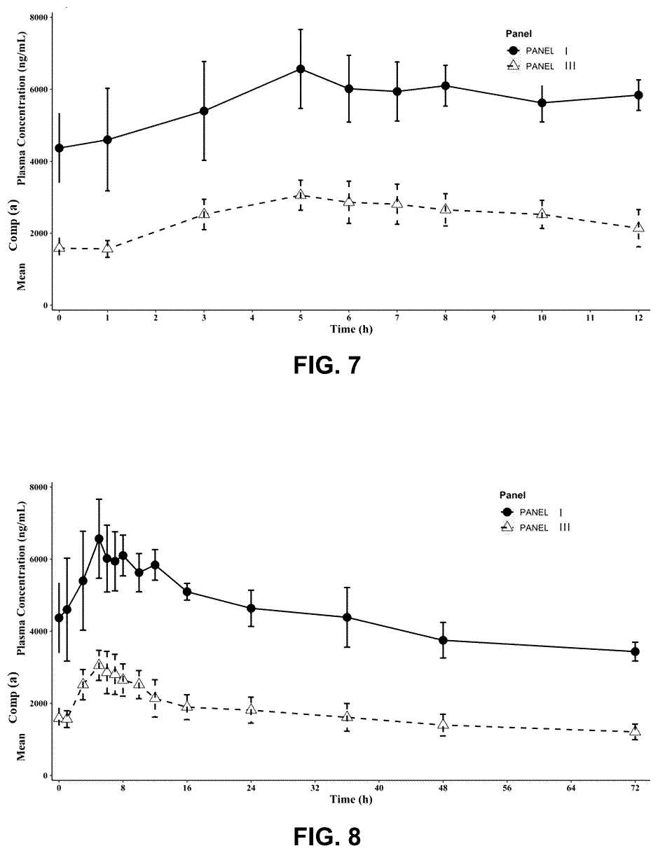

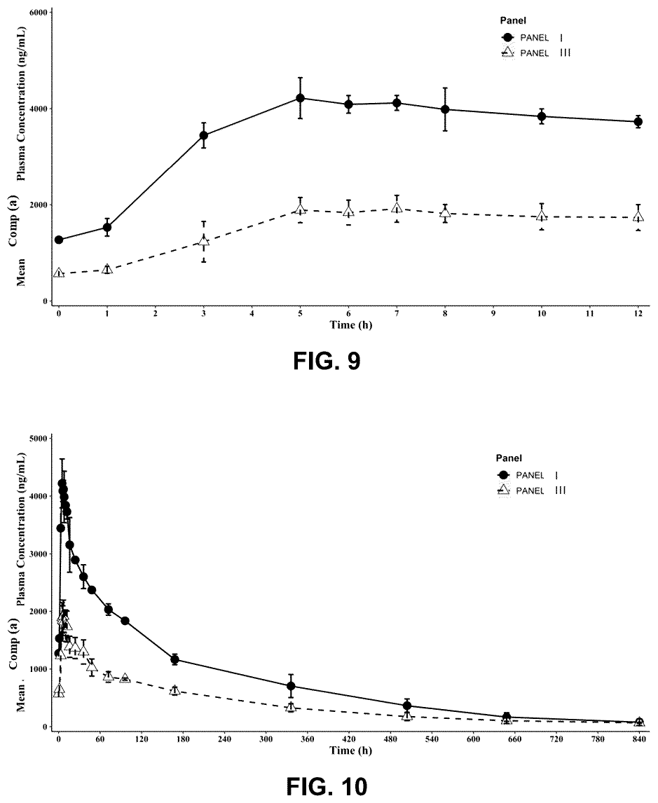

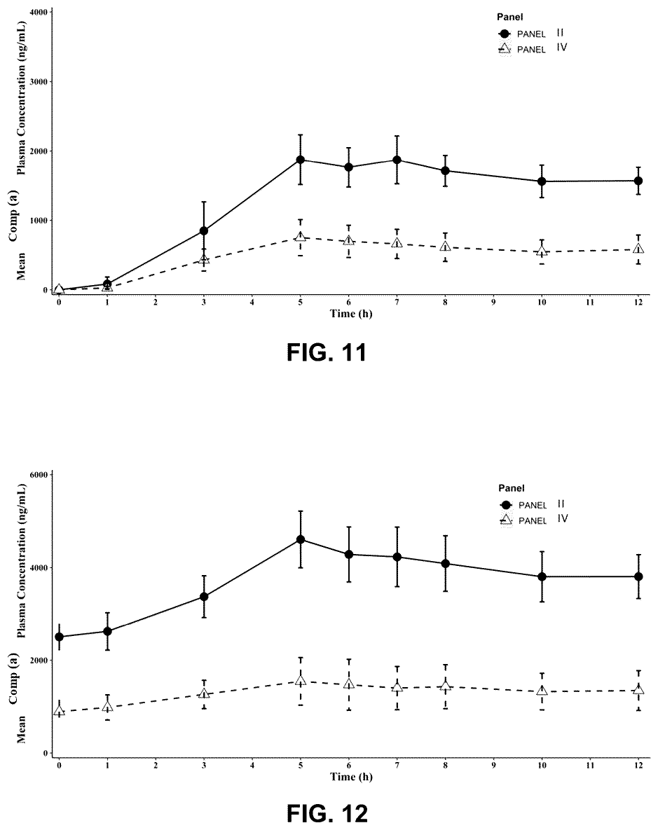

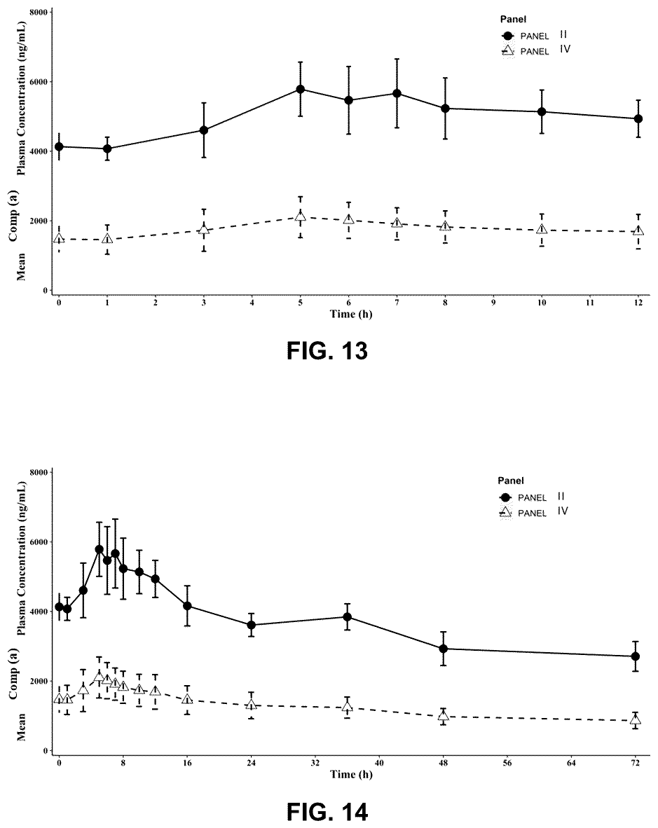

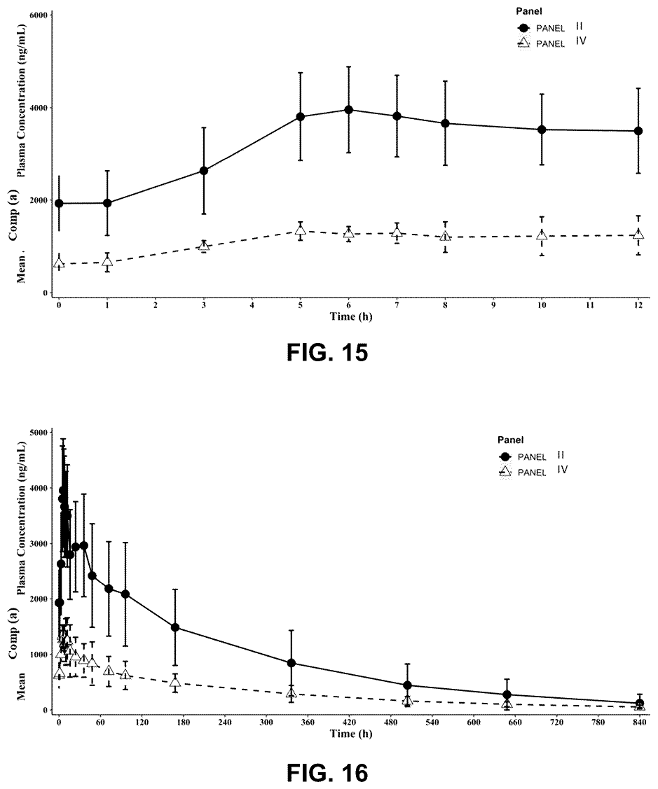

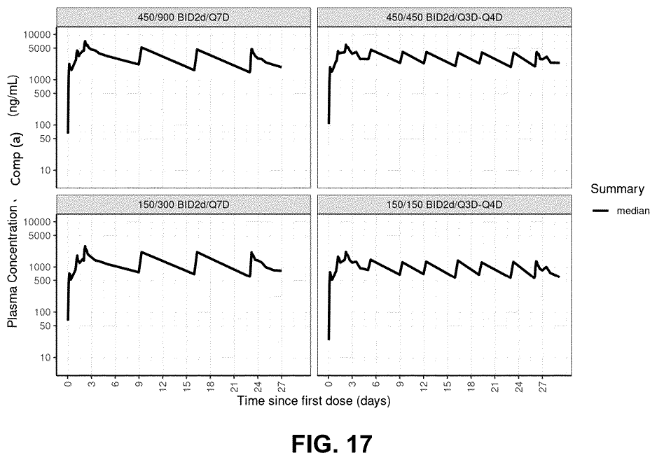

The PK parameters were calculated in the same manner as described in Example 1. Table 7A shows the day/plasma PK parameters of Panels I and III, Table 7B shows the day/plasma PK parameters for Panels II and IV. FIGS. 5 to 10 show the plasma concentration of Compound (a) on subjects in Panels I and II. FIGS. 11 to 16 show the plasma concentration of Compound (a) on subjects in Panels II and IV. FIG. 17 shows the plasma concentration of Compound (a) on subjects in Panels 1, 2, 3 and 4. FIGS. 5 to 10 and 11 to 16 were based on preliminary data analysis, whereas the graphs in FIG. 17 was based on a final data analysis. Hence, the graphs depicted in FIG. 17 were generated using data from a later time point than the timepoints at which the data shown in Tables 7A and 7B was generated.

TABLE 7A
Day/plasma PK parameters of Panels I and III

| | Panel I | Panel III |
|---|---|---|
| Day 1 | | |
| n | 2 | 6 |
| Mean Cₘₐₓ | 2015 (658) | 741 (126) |
| (ng/mL) (SD) | | |
| Mean Cₘᵢₙ | 0.000 (0.000) | 0.000 (0.000) |
| (ng/mL) (SD) | | |
| Median tₘₐₓ | 7.50 (7.00-8.00) | 5.00 (5.00-6.00) |
| (h) (min-max) | | |
| Mean AUC_{τ} | 17464 (5375) | 5676 (868) |
| (h*ng/mL) (SD) | | |
| Day 2 | | |
| n | 2 | 6 |
| Mean Cₘₐₓ | 5075 (1322) | 1813 (323) |
| (ng/mL) (SD) | | |
| Mean Cₘᵢₙ | 2750 (1089) | 899 (151) |
| (ng/mL) (SD) | | |
| Median tₘₐₓ | 5.50 (5.00-6.00) | 5.00 (5.00-6.00) |
| (h) (min-max) | | |
| Mean AUC_{τ} | 46458 (12555) | 16758 (2744) |
| (h*ng/mL) (SD) | | |
| Mean C_{trough} | 2780 (1047) | 899 (151) |
| (ng/mL) (SD) | | |
| Day 3 | | |
| n | 2 | 6 |
| Mean Cₘₐₓ | 6565 (1096) | 3098 (475) |
| (ng/mL) (SD) | | |
| Median tₘₐₓ | 5.00 (5.00-5.00) | 5.00 (5.00-6.00) |
| (h) (min-max) | | |
| Mean AUC_{24 h} | 128718 (15355) | 52448 (8235) |
| (h*ng/mL) (SD) | | |
| Mean AUC_{72 h} | 317868 (40344) | 122317 (22421) |
| (h*ng/mL) (SD) | | |
| Mean C_{72 h} | 3435 (262) | 1209 (217) |
| (ng/mL) (SD) | | |
| Mean C_{trough} | 4370 (976) | 1582 (298) |
| (ng/mL) (SD) | | |
| Day 10 | | |
| n | 2 | 6 |
| Mean Cₘₐₓ | 4235 (1167) | 2182 (257) |
| (ng/mL) (SD) | | |
| Mean C_{trough} | 1965 (35.4) | 775 (99.3) |
| (ng/mL) (SD) | | |
| Day 17 | | |
| n | 2 | 6 |
| Mean Cₘₐₓ | 4280 (948) | 2052 (255) |
| (ng/mL) (SD) | | |
| Mean C_{trough} | 1500 (113) | 700 (98.1) |
| (ng/mL) (SD) | | |
| Day 24 | | |
| n | 2 | 5 |
| Mean Cₘₐₓ | 4265 (361) | 2134 (284) |
| (ng/mL) (SD) | | |
| Median tₘₐₓ | 6.00 (5.00-7.00) | 7.00 (5.00-12.0) |
| (h) (min-max) | | |
| Mean AUC_{24 h} | 79718 (5752) | 37249 (4711) |
| (h*ng/mL) (SD) | | |
| Mean AUC_{72 h} | 195458 (6092) | 92482 (12992) |
| (h*ng/mL) (SD) | | |
| Mean AUC_{∞} | 675902 (93433) | 325179 (40058)^{a} |
| (h*ng/mL) (SD) | | |
| Mean AUCₗₐₛₜ | 657393 (75085) | 299205 (38398) |
| (h*ng/mL) (SD) | | |

C_{trough} measurements are before morning and evening dose.
SD Standard deviation.

TABLE 7B
Day/plasma PK parameters of Panels II and IV

| | Panel II | Panel IV |
|---|---|---|
| Day 1 | | |
| n | 6 | 7 |
| Mean Cₘₐₓ | 1965 (307) | 753 (260) |
| (ng/mL) (SD) | | |
| Mean Cₘᵢₙ | 0.000 (0.000) | 0.000 (0.000) |
| (ng/mL) (SD) | | |
| Median tₘₐₓ | 5.50 (5.00-7.00) | 5.00 (5.00-6.00) |
| (h) (min-max) | | |
| Mean AUC_{τ} | 15528 (2411) | 5977 (1950) |
| (h*ng/mL) (SD) | | |
| Day 2 | | |
| n | 6 | 7 |
| Mean Cₘₐₓ | 4643 (592) | 1600 (521) |
| (ng/mL) (SD) | | |
| Mean Cₘᵢₙ | 2492 (307) | 892 (257) |
| (ng/mL) (SD) | | |
| tₘₐₓ (h) | 5.00 (5.00-8.00) | 5.00 (5.00-12.0) |
| Mean AUC_{τ} | 44913 (5805) | 15777 (4660) |
| (h*ng/mL) (SD) | | |
| Mean C_{trough} | 2503 (293) | 892 (257) |
| (ng/mL) (SD) | | |
| Day 3 | | |
| n | 6 | 6 |
| Mean Cₘₐₓ | 5952 (808) | 2118 (579) |
| (ng/mL) (SD) | | |
| Median tₘₐₓ | 6.50 (5.00-12.0) | 5.00 (5.00-7.00) |
| (h) (min-max) | | |
| Mean AUC_{24 h} | 109522 (11705) | 38629 (10833) |
| (h*ng/mL) (SD) | | |
| Mean AUC_{72 h} | 262512 (26542) | 89224 (23496) |
| (h*ng/mL) (SD) | | |
| Mean C_{72 h} | 2708 (429) | 865 (236) |
| (ng/mL) (SD) | | |
| Mean C_{trough} | 4132 (401) | 1472 (385) |
| (ng/mL) (SD) | | |
| Day 6 | | |
| n | 6 | 6 |
| Mean Cₘₐₓ | 4382 (503) | 1455 (410) |
| (ng/mL) (SD) | | |
| Mean C_{trough} | 2708 (429) | 865 (236) |
| (ng/mL) (SD) | | |
| Day 10 | | |
| n | 6 | 6 |
| Mean Cₘₐₓ | 4083 (443) | 1232 (391) |
| (ng/mL) (SD) | | |
| Mean C_{trough} | 2340 (577) | 681 (234) |
| (ng/mL) (SD) | | |
| Day 13 | | |
| n | 6 | 6 |
| Mean Cₘₐₓ | 4068 (670) | 1185 (356) |
| (ng/mL) (SD) | | |
| Mean C_{trough} | 2343 (639) | 689 (238) |
| (ng/mL) (SD) | | |
| Day 17 | | |
| n | 5 | 4 |
| Mean Cₘₐₓ | 3942 (710) | 1209 (315)^{a} |
| (ng/mL) (SD) | | |
| Mean C_{trough} | 2088 (693) | 570 (269) |
| (ng/mL) (SD) | | |
| Day 20 | | |
| n | 5 | 3 |
| Mean Cₘₐₓ | 4062 (729) | 1201 (285) |
| (ng/mL) (SD) | | |
| Mean C_{trough} | 2270 (652) | 710 (236) |
| (ng/mL) | | |
| Day 24 | | |
| n | 5 | 3 |
| Mean Cₘₐₓ | 3916 (771) | 1257 (337) |
| (ng/mL) (SD) | | |
| Mean C_{trough} | 2016 (847) | 612 (239) |
| (ng/mL) (SD) | | |
| Day 27 | | |
| n | 5 | 3 |
| Mean Cₘₐₓ | 3980 (906) | 1383 (292) |
| (ng/mL) (SD) | | |
| Median tₘₐₓ | 6.00 (5.00-6.00) | 5.00 (5.00-12.0) |
| (h) (min-max) | | |
| Mean AUC_{24 h} | 74221 (19998) | 25998 (7983) |
| (h*ng/mL) (SD) | | |
| Mean AUC_{72 h} | 197209 (62345) | 65776 (23960) |
| (h*ng/mL) (SD) | | |
| Mean AUC_{∞} | 808837 (470444) | 274422 (118487) |
| (h*ng/mL) (SD) | | |
| Mean AUCₗₐₛₜ | 771143 (416180) | 258090 (107553) |
| (h*ng/mL) (SD) | | |
| Mean C_{trough} | 1928 (605) | 623 (237) |
| (ng/mL) (SD) | | |

^{a}n = 3
C_{trough} measurements are before morning and evening dose.
SD Standard deviation.

Safety and tolerability of Compound (a) was evaluated throughout the study, as described in Example 1.

### Overall Reported Events:

4 Grade 2 Treatment Emergent Events.
No Grade 3 Treatment Emergent Events.

### Related Adverse Events (AEs) Reported

Panel I—(loading dose (LD) of 450 mg twice daily for two days, followed by a maintenance dose (MD) of 900 mg once a week (Q7D))—no related AEs.
Panel II—(a LD of 450 mg twice daily on for two days, followed by a MD of 450 mg once every 3 (Q3D) or 4 days (Q4D))—one Grade 1 AE of skin burning sensation considered related.
Panel III—(LD of 150 mg twice daily for two days, followed by a MD of 300 mg once a week (Q7D))—no related AEs.
Panel IV—(LD of 150 mg twice daily for two days, followed by a MD of 150 mg once every 3 (Q3D) or 4 days (Q4D))—two Grade 1 AE of pruritic rash considered related.
   No serious adverse events (SAEs) or/and serious unexpected adverse reactions (SUSARs) were observed.
   The following conclusions may be drawn.
It is feasible to maintain Compound (a) exposures over time with weekly or twice weekly dosing.
Compound (a) was safe and well tolerated.
Considerations for weekly dosing of Compound (a):
   Higher peak-to-trough ratio compared to daily dosing.
   Importance of therapy/food compliance.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention and that embodiments within the scope of these claims and their equivalents be covered thereby.

1. A method of preventing and/or treating dengue viral infections, wherein said method comprises the administration of a compound of formula (I) in an oral dosage form to a human in a fed or fasted state and wherein compound of formula (I) corresponds to 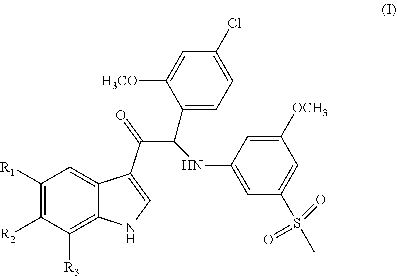
a stereo-isomeric form, a pharmaceutically acceptable salt, solvate or polymorph thereof;
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃.

2. The method according to claim 1, wherein the compound is administered in an oral dosage form to a human in a fed state.

3. The method according to claim 2, wherein said human is in a fed state before or simultaneously with the administration of said oral dosage form.

4. The method according to claim 1, wherein said human has eaten at most 3 hours before the time of administration of the oral dosage form.

5. The method according to claim 1, wherein the oral dosage form is administered to the human at least once a day, at least twice a day, at least three times a day, or at least four times a day.

6. The method according to claim 1, wherein the oral dosage form is a solid dosage form.

7. The method according to claim 1, wherein the oral dosage form is administered following a dosage regimen comprising:
1) at least one loading phase during which at least one dose (A) of compound of formula (I) is administered at least once a day over a time period of at least one day; and
2) at least one maintenance phase starting on the next day following the last day of administration of dose (A) or the last day of the loading phase, during which at least one dose (B) of compound of formula (I), is administered at least once a day over a time period of at least one day:
wherein dose (A) is either higher or lower than dose (B), preferably, dose (A) is higher than dose (B).

8. The method according to claim 1 wherein the oral dosage form, is a solid dosage form comprising a cellulose derivative having a viscosity ranging between 3 and 5000 mPa·s in 2 wt % solution in H2O at 25° C. and is selected from the group comprising HPMC E5, HPMC E6, HPMC E15, HPMC E50, HPMC K4M HPMC-AS and any combination thereof.

9. The method according to claim 1, wherein the oral dosage form is formulated in a pharmaceutical formulation comprising from 0.5 mg to 1500 mg of compound of formula (I); preferably the formulation comprises from 1 mg to 1000 mg of compound of formula (I); preferably the formulation comprises from 2 mg to 900 mg compound of formula (I).

10. The method according to claim 1, wherein the compound of formula (I) is: 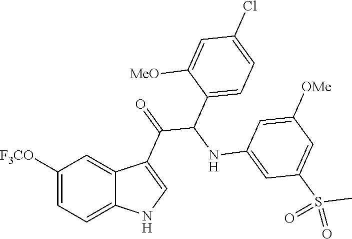 [SMILES: COc1cc(NC(C(=O)c2c[nH]c3ccc(OC(F)(F)F)cc23)c2ccc(Cl)cc2OC)cc(S(C)(=O)=O)c1]
or a stereo-isomeric form, a pharmaceutically acceptable salt, solvate or polymorph thereof.

11. A method of preventing and/or treating dengue viral infection, said method comprising the administration of a pharmaceutical formulation comprising
a) a compound formula (I); and
b1) methacrylic acid copolymer, or
b2) hydroxypropyl methylcellulose (HPMC);
wherein the pharmaceutical formulation is administered in an oral dosage form to a human in either a fed or fasted state; and wherein formula (I) corresponds to 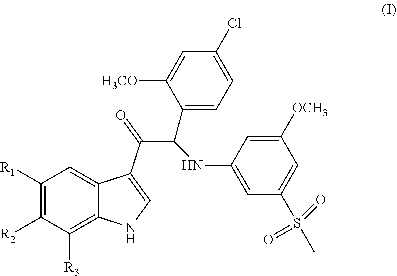
a stereo-isomeric form, a pharmaceutically acceptable salt, solvate or polymorph thereof:
said compound is selected from the group wherein:
R₁ is H, R₂ is F and R₃ is H or CH₃,
R₁ is H, CH₃ or F, R₂ is OCH₃ and R₃ is H,
R₁ is H, R₂ is OCH₃ and R₃ is CH₃,
R₁ is CH₃, R₂ is F and R₃ is H,
R₁ is CF₃ or OCF₃, R₂ is H and R₃ is H,
R₁ is OCF₃, R₂ is OCH₃ and R₃ is H,
R₁ is OCF₃, R₂ is H and R₃ is CH₃.

12. The method according to claim 11, wherein the pharmaceutical formulation is administered in an oral dosage form to a human in a fed state.

13. The method according to claim 11, wherein said human is in a fed state before or during the administration of said oral dosage form.

14. The method according to claim 11, wherein said human is in a fed state and has eaten at most 3 hours before the time of administration of the oral dosage form.

15. The method according to claim 11, wherein the oral dosage form is administered following a dosage regimen comprising:
1) at least one loading phase during which at least one dose (A) of compound of formula (I) is administered at least once a day over a time period of at least one day; and
2) at least one maintenance phase starting on the next day following the last day of administration of dose (A) or the last day of the loading phase, during which at least one dose (B) of compound of formula (I), is administered at least once a day over a time period of at least one day:
wherein dose (A) is either higher or lower than dose (B), preferably, dose (A) is higher than dose (B).
